(19) United States
(12) Patent Application Publication
(10) Pub. No.: US 2021/0145841 A1
(43) Pub. Date: May 20, 2021

(54) ENHANCEMENT OF DELIVERY OF LIPOPHILIC ACTIVE AGENTS ACROSS THE BLOOD-BRAIN BARRIER AND METHODS FOR TREATING CENTRAL NERVOUS SYSTEM DISORDERS

(71) Applicant: POVIVA CORP., Carson City, NV (US)

(72) Inventors: John Docherty, Port Perry (CA); Christopher Andrew Bunka, Kelowna (CA)

(21) Appl. No.: 17/047,479

(22) PCT Filed: Apr. 16, 2019
(86) PCT No.: PCT/US2019/027769

§ 371 Date: Oct. 14, 2020

Related U.S. Application Data
(60) Provisional application No. 62/689,096, filed on Jun. 23, 2018.
(60) Provisional application No. 62/748,520, filed on Oct. 21, 2018.

(51) Int. Cl. A61K 31/5415; A61K 31/465; A61K 31/167; A61K 31/192; A61K 31/196; A61K 31/355; A61K 31/616; A61K 9/00; A23F 3/40

(52) U.S. Cl. CPC A61K 31/5415; A61K 31/465; A61K 31/167; A61K 31/192; A23F 3/405; A61K 31/355; A61K 31/616; A61K 9/0095; A61K 31/196

(57) ABSTRACT

Aspects described herein relate to edible compositions and methods for the enhancement of delivery of lipophilic active agents across the blood-brain barrier, particularly wherein the lipophilic active agent infused edible compositions produce greater concentrations of lipophilic active agents in subjects' central nervous system tissues as compared to control compositions. Further provided are methods for treating central nervous system disorders comprising administering the edible compositions to subjects in need thereof.

### CROSS-REFERENCE TO RELATED APPLICATIONS

This application is a PCT International Application which claims the benefit of U.S. Provisional Application No. 62/689,096, filed Jun. 23, 2018; and U.S. Provisional Application No. 62/748,520, filed Oct. 21, 2018; each of which are incorporated herein by reference in their entireties.

### TECHNICAL FIELD

Aspects described herein relate to edible compositions and methods for the enhancement of delivery of lipophilic active agents across the blood-brain barrier, particularly wherein the lipophilic active agent infused edible compositions produce greater concentrations of lipophilic active agents in subjects' central nervous system tissues as compared to control compositions.

### BACKGROUND

The blood-brain barrier protects the brain by keeping it isolated from harmful toxins in the blood stream. The blood-brain barrier is formed by special tight junctions between the epithelial cells that surround the brain tissue and prevent large molecules as well as many ions from passing between the junction spaces. In order to enter the brain tissue, such large molecules and ions are forced to go through the endothelial cells, and therefore must pass through the cell membranes of the endothelial cells (Rubin & Staddon (1999) Ann. Rev. Neurosci. 22:11-28). Accordingly, molecules that are easily able to transverse the blood-brain barrier tend to be very lipophilic, though other molecules such as glucose, oxygen, and carbon dioxide are actively transported across the barrier to support normal cellular function of the brain (Ramlakhan & Altman (1990) New Scientist, 128:5).

Although lipophilicity is generally associated with molecules that are easily able to cross the blood-brain barrier, lipophilicity is not the leading characteristic for molecules that transverse the blood-brain barrier. Seelig and colleagues studied the association of different factors with the ability of molecules to diffuse across the blood-brain barrier, including lipophilicity, Gibbs Adsorption Isotherm, a Co CMC Plot, and the surface area of the drug to water and air (Seelig et al. (1994) Proc. Nat. Acad. Sci. (USA) 91:68-72). Their results showed that barrier permittivity is based on a complex interaction between relative size and the surface activity of the molecule, in which the surface activity includes the molecular properties of both hydrophobic and charged residues (Seelig et al. (1994) Proc. Nat. Acad. Sci. (USA) 91:68-72).

The delivery of drugs to the brain is also complicated by the fact that some compounds that cross the blood-brain barrier do so in a way that does not result in therapeutically effective amounts of these compounds in brain tissue (Dadparvar et al. (2011) Toxicology Letters 206:60-66). In some cases, the bioavailability of the compound in the blood stream is so low that only a small amount of the compound is available to pass through the blood-brain barrier (Dadparvar et al. (2011) Toxicology Letters 206:60-66). In other cases, the compound may pass through the blood-brain barrier, but not in sufficient concentrations to overcome degradative processes within brain tissues that render the compound inactive (Dadparvar et al. (2011) Toxicology Letters 206:60-66).

Therefore, there is a need for improved compositions and methods for the enhancement of delivery of lipophilic active agents across the blood-brain barrier.

### SUMMARY

To address the foregoing problems, in whole or in part, and/or other problems that may have been observed by persons skilled in the art, the present disclosure provides compositions and methods as described by way of example as set forth below.

A process is provided for making an edible product infused with a lipophilic active agent with enhanced delivery across the blood brain barrier in a subject, comprising:
(a) providing a therapeutically effective amount of a lipophilic active agent;
(b) providing a bioavailability enhancing agent;
(c) providing an edible substrate;
(d) contacting the edible substrate with an oil comprising the lipophilic active agent and the bioavailability enhancing agent; and
(e) dehydrating the edible substrate;
   thereby producing the edible product infused with a lipophilic active agent with enhanced delivery across the blood brain barrier in the subject; wherein the bioavailability enhancing agent comprises an edible oil comprising long chain fatty acids and/or medium chain fatty acids that enhance the bioavailability of the lipophilic active agent and enhance delivery across the blood brain barrier in the subject.

In some embodiments, the edible product is selected from the group consisting of a pill, tablet, lozenge, mini lozenge, capsule, caplet, pouch, gum, spray, food, and combinations thereof.

In some embodiments, the edible substrate is selected from the group consisting of inulin, starch, modified starches, xanthan gum, carboxymethyl cellulose, methyl cellulose, hydroxypropylmethyl cellulose, konjac, chitosan, tragacanth, karaya, ghatti, larch, carageenan, alginate, chemically modified alginate, agar, guar, locust bean, psyllium, tara, gellan, curdlan, pullan, gum arabic, gelatin, pectin, and combinations thereof.

In some embodiments, the edible product further comprises a flavoring agent selected from the group consisting of vanilla, vanillin, ethyl vanillin, orange oil, fruit and berry type flavorants, Dramboui, bourbon, scotch, whiskey, spearmint, lavender, cinnamon, chai, cardamon, apium graveolents, clove, cascarilla, nutmeg, sandalwood, bergamot, geranium, honey essence, rose oil, lemon oil, Japanese mint, cassia, caraway, cognac, jasmin, chamomile, menthol, ylang ylang, sage, fennel, pimenta, ginger, anise, chai, coriander, coffee, peppermint, wintergreen, mint oils from a species of the genus Mentha, and combinations thereof.

In some embodiments, the edible product further comprises an additive selected from the group consisting of a non-nicotine alkaloid, a mineral, a vitamin, a dietary supplement, a dietary mineral, a nutraceutical, an energizing agent, a soothing agent, a coloring agent, an amino acid, a chemsthetic agent, an antioxidant, a food grade emulsifier, a pH modifier, a botanical, a teeth whitening agent, a therapeutic agent, a sweetener, a flavorant, and combinations thereof.

In some embodiments, the bioavailability of the lipophilic active agent in a subject is at least 2 times, 5 times, or 10 times greater than the bioavailability of the lipophilic active agent in the subject in the absence of the edible oil comprising long chain fatty acids and/or medium chain fatty acids. In some embodiments, the concentration of lipophilic active agent in central nervous system tissue of the subject is at least 1.5 times, 2 times, or 5 times greater than the concentration of lipophilic active agent in central nervous system tissue in the subject in the absence of the edible oil comprising long chain fatty acids and/or medium chain fatty acids.

In some embodiments, the edible oil comprising long chain fatty acids and/or medium chain fatty acids is substantially free of omega-6 fatty acids.

In some embodiments, the long chain fatty acids and/or medium chain fatty acids are selected from the group consisting of oleic acid, undecanoic acid, valeric acid, heptanoic acid, pelargonic acid, capric acid, lauric acid, and eicosapentaenoic acid.

In some embodiments, the lipophilic active agent is selected from the group consisting of: cannabinoids, terpenes and terpenoids, non-steroidal anti-inflammatory drugs (NSAIDs), vitamins, nicotine or an analog thereof, phosphodiesterase 5 (PDE5) inhibitors, Maca extract, hormones, fentanyl or an analog thereof, buprenorphine or an analog thereof, scopolamine or an analog thereof, antioxidants, a nicotine compound, and an imaging agent.

In some embodiments, the cannabinoid is a psychoactive cannabinoid.

In some embodiments, the cannabinoid is a non-psychoactive cannabinoid.

In some embodiments, the NSAID is acetylsalicylic acid, ibuprophen, acetaminophen, diclofenac, indomethacin, piroxicam, or a COX inhibitor.

In some embodiments, the vitamin is vitamin A, D, E, or K.

In some embodiments, the PDE5 inhibitor is avanafil, lodenafil, mirodenafil, sildenafil, tadalafil, vardenafil, udenafil, acetildenafil, thiome-thisosildenafil, or analogs thereof.

In some embodiments, the hormone is an estrogen, an anti-estrogen, an androgen, an anti-androgen, or a progestin.

In some embodiments, the antioxidant is astaxanthin, Superoxide Dismusase, beta-carotene, selenium, lycopene, lutein, Coenzyme Q10, phytic acid, flavonoids, a polyphenol, a substituted 1,2-dihydroquinoline, ascorbic acid and its salts, ascorbyl palmitate, ascorbyl stearate, anoxomer, N-acetylcysteine, benzyl isothiocyanate, o-, m- or p-amino benzoic acid (o is anthranilic acid, p is PABA), butylated hydroxyanisole (BHA), butylated hydroxytoluene (BHT), caffeic acid, canthaxantin, alpha-carotene, beta-carotene, beta-caraotene, beta-apo-carotenoic acid, carnosol, carvacrol, catechins, cetyl gallate, chlorogenic acid, citric acid and its salts, clove extract, coffee bean extract, p-coumaric acid, 3,4-dihydroxybenzoic acid, N,N′-diphenyl-p-phenylenediamine (DPPD), dilauryl thiodipropionate, distearyl thiodipropionate, 2,6-di-tert-butylphenol, dodecyl gallate, edetic acid, ellagic acid, erythorbic acid, sodium erythorbate, esculetin, esculin, 6-ethoxy-1,2-dihydro-2,2,4-trimethylquinoline, ethyl gallate, ethyl maltol, ethylenediaminetetraacetic acid (EDTA), eucalyptus extract, eugenol, ferulic acid, flavonoids, flavones (e.g., apigenin, chrysin, luteolin), flavonols (e.g., datiscetin, myricetin, daemfero), flavanones, fraxetin, fumaric acid, gallic acid, gentian extract, gluconic acid, glycine, gum guaiacum, hesperetin, alpha-hydroxybenzyl phosphinic acid, hydroxycinammic acid, hydroxyglutaric acid, hydroquinone, N-hydroxysuccinic acid, hydroxytryrosol, hydroxyurea, ice bran extract, lactic acid and its salts, lecithin, lecithin citrate; R-alpha-lipoic acid, lutein, lycopene, malic acid, maltol, 5-methoxy tryptamine, methyl gallate, monoglyceride citrate; monoisopropyl citrate; morin, beta-naphthoflavone, nordihydroguaiaretic acid (NDGA), octyl gallate, oxalic acid, palmityl citrate, phenothiazine, phosphatidylcholine, phosphoric acid, phosphates, phytic acid, phytylubichromel, pimento extract, propyl gallate, polyphosphates, quercetin, trans-resveratrol, rosemary extract, rosmarinic acid, sage extract, sesamol, silymarin, sinapic acid, succinic acid, stearyl citrate, syringic acid, tartaric acid, thymol, tocopherols (i.e., alpha-, beta-, gamma- and delta-tocopherol), tocotrienols (i.e., alpha-, beta-, gamma- and delta-tocotrienols), tyrosol, vanilic acid, 2,6-di-tert-butyl-4-hydroxymethylphenol (i.e., lonox 100), 2,4-(tris-3′,5′-bi-tert-butyl-4′-hydroxybenzyl)-mesitylene (i.e., lonox 330), 2,4,5-trihydroxybutyrophenone, ubiquinone, tertiary butyl hydroquinone (TBHQ), thiodipropionic acid, trihydroxy butyrophenone, tryptamine, tyramine, uric acid, vitamin K and derivates, vitamin Q10, wheat germ oil, zeaxanthin, or combinations thereof.

In some embodiments, the nicotine compound is selected from the group consisting of nicotine, free base nicotine, pharmacologically acceptable salts of nicotine, a nicotine complex, and polymer resins of nicotine, wherein the polymer resin is selected from the group consisting of nicotine polacrilex and nicotine resinate.

In some embodiments, the edible product is a food product and the edible substrate is selected from the group consisting of tea leaves, coffee beans, cocoa powder, meats, fish, fruits, vegetables, dairy products, legumes, pastas, breads, grains, seeds, nuts, spices, and herbs.

In some embodiments, the bioavailability enhancing agent is a protective colloid, an edible oil or fat, and a lipophilic active agent taste masking agent. In some embodiments, the bioavailability enhancing agent that is a protective colloid, an edible oil or fat, and a lipophilic active agent taste masking agent is nonfat dry milk. In some embodiments, the edible product is lyophilized.

A process is also provided for making a beverage product infused with a lipophilic active agent obtainable by the steps of:
(i) providing the edible product infused with a lipophilic active agent according to any of the processes described above, wherein the edible product infused with a lipophilic active agent is tea leaves, coffee beans, or cocoa powder infused with a lipophilic active agent; and
(ii) steeping the tea leaves, coffee beans, or cocoa powder infused with a lipophilic active agent in a liquid;
   thereby producing the beverage product infused with the lipophilic active agent.

A method is also provided for treating a central nervous system disease, disorder, or condition comprising administering the edible product infused with a lipophilic active agent or the beverage product infused with a lipophilic active agent to a subject in need thereof, and wherein the central nervous system disease, disorder, or condition is selected from the group consisting of a metabolic disease, a behavioral disorder, a personality disorder, dementia, a cancer, a neurodegenerative disorder, pain, a viral infection, a sleep disorder, and an arteriovenous malformation, a brain aneurysm, a brain tumor, a spinal cord tumor, facial paralysis, a pituitary disorder, a stroke, and a seizure disorder.

In some embodiments, the central nervous system disease, disorder, or condition is selected from the group consisting of Amyotrophic Lateral Sclerosis (ALS), Multiple Sclerosis, Parkinson's Disease, Fabry disease, Wernicke-Korsakoff syndrome, Alzheimer's disease, Huntington's disease, Lewy Body disease, Canavan disease, Hallervorden-Spatz disease, and Machado-Joseph disease.

In some embodiments, the central nervous system disease, disorder, or condition is selected from the group consisting of acid lipase disease, attention deficit hyperactivity disorder (ADHD), an anxiety disorder, borderline personality disorder, bipolar disorder, depression, an eating disorder, obsessive-compulsive disorder, schizophrenia, Barth syndrome, Tourette's syndrome, and Restless Leg syndrome.

In some embodiments, the pain is selected from the group consisting of neuropathic pain, central pain syndrome, somatic pain, visceral pain, and headache.

A method is also provided for enhancing the delivery of a lipophilic agent across the blood brain barrier of a subject, comprising administering the edible product infused with a lipophilic active agent or the beverage product infused with a lipophilic active agent to a subject in need thereof. In some embodiments, the edible product or the beverage product is heated to a temperature that is greater than or equal to human body temperature.

A kit is also provided comprising the edible product or the beverage product as described above and instructions for use thereof.

Other compositions, methods, features, and advantages of the invention will be or will become apparent to one with skill in the art upon examination of the following figures and detailed description. It is intended that all such additional compositions, methods, features, and advantages be included within this description, be within the scope of the invention, and be protected by the accompanying claims.

### BRIEF DESCRIPTION OF THE DRAWINGS

The invention can be better understood by referring to the following figures. The components in the figures are not necessarily to scale, emphasis instead being placed upon illustrating the principles of the invention.

FIG. 1 shows results from Example 5 comparing nicotine concentrations in various tissues following administration of DEHYDRATECH™ and control compositions in rats.

FIG. 2 shows results from Example 6 showing improvement in peak nicotine blood levels following administration of DEHYDRATECH™ and control compositions in rats.

FIG. 3 shows results from Example 6 comparing nicotine concentrations in various tissues following administration of DEHYDRATECH™ and control compositions in rats.

FIG. 4 shows results from Examples 5 and 6 comparing improvements in maximum brain concentration, time to Cmax, and total quantity in brain tissue following administration of DEHYDRATECH™ and control compositions in rats.

### DETAILED DESCRIPTION

The presently disclosed subject matter now will be described more fully hereinafter. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims.

In some embodiments, the compositions or methods comprise the specified components or steps. In some embodiments, the compositions or methods consist of the specified components or steps. In other embodiments, the compositions or methods consist essentially of the specified components or steps. As used herein, “consists essentially of” the specified components or steps means that the composition includes at least the specified components or steps, and may also include other components or steps that do not materially affect the basic and novel characteristics of the invention.

Aspects described herein relate to edible products, such as food and beverage compositions, and methods for the enhancement of delivery of lipophilic active agents across the blood-brain barrier, particularly wherein the lipophilic active agent infused edible products produce greater concentrations of lipophilic active agents in subjects' central nervous system tissues as compared to control compositions. The present invention is also directed to lipophilic active agent infused edible products that provide enhanced bioavailability in a subject, particularly wherein the unpleasant taste of the lipophilic active agent is masked. Processes for making the edible products are provided, as well as methods for treating central nervous system disorders, diseases, or conditions comprising administering any of the compositions disclosed herein to a subject in need thereof.

It was surprisingly found that the lipid content and colloidal properties of the lipophilic active agent infused compositions increased the bioavailability of the lipophilic active agents in subjects as compared to typical oral ingestion of the lipophilic active agents. The lipophilic active agent infused compositions also allow for enhanced oral bioavailability associated with the co-administration of lipophilic drugs and lipid containing foods without the dosing and compliance problems associated with administration of the lipophilic active agent in a separate formulation from the foods and beverages. In addition, lipophilic active agents are surprisingly more effectively transported through the intestinal mucosa when combined with foods and beverages that also contain antioxidants such as black tea, thereby producing a synergistic effect on lipophilic active agent absorption and bioavailability.

The present invention also relates, in part, to lipophilic active agent infused compositions comprising one or more lipophilic active agent taste masking agents. Many lipophilic active agents have unpalatable taste profiles, which could hinder the use of orally ingested lipophilic active agents as therapeutic treatments. In one aspect, it was surprisingly found that dry milk used within the processes for making the lipophilic active agent infused edible compositions of the invention acted as both a bioavailability enhancing agent as well as a lipophilic active agent taste masking agent.

It was further surprisingly found that edible oils comprising long chain fatty acids and/or medium chain fatty acids acted as bioavailability enhancing agents for lipophilic active agents within the compositions and methods disclosed herein.

### I. Processes and Methods

A. Delivery of Active Agents Across the Blood Brain Barrier

The blood-brain barrier (BBB), while providing effective protection to the brain against circulating toxins, also creates major difficulties in the pharmacological treatment of brain diseases such as Alzheimer's disease, Parkinson's disease, and brain cancer. Most charged molecules, and most molecules over 700 Daltons in size, are unable to pass through the barrier, and smaller molecules may be conjugated in the liver. These factors create major difficulties in the pharmacological treatment of diseases of the brain and central nervous system (CNS), such as Alzheimer's disease, Parkinson's disease, bacterial and viral infections and cancer.

Many therapeutic agents for the treatment of diseases and disorders of the brain and CNS are sufficiently hydrophilic to preclude direct transport across the BBB. Furthermore, these drugs and agents are susceptible to degradation in the blood and peripheral tissues that increase the dose necessary to achieve a therapeutically effective serum concentration. However, as described above, although lipophilicity is generally associated with molecules that are easily able to cross the blood-brain barrier, lipophilicity is not the leading characteristic for molecules that transverse the blood-brain barrier. Seelig and colleagues studied the association of different factors with the ability of molecules to diffuse across the blood-brain barrier, including lipophilicity, Gibbs Adsorption Isotherm, a Co CMC Plot, and the surface area of the drug to water and air (Seelig et al. (1994) Proc. Nat. Acad. Sci. (USA) 91:68-72). Their results showed that barrier permittivity is based on a complex interaction between relative size and the surface activity of the molecule, in which the surface activity includes the molecular properties of both hydrophobic and charged residues (Seelig et al. (1994) Proc. Nat. Acad. Sci. (USA) 91:68-72).

The delivery of drugs to the brain is also complicated by the fact that some compounds that cross the blood-brain barrier do so in a way that does not result in therapeutically effective amounts of these compounds in brain tissue (Dadparvar et al. (2011) Toxicology Letters 206:60-66). In some cases, the bioavailability of the compound in the blood stream is so low that only a small amount of the compound is available to pass through the blood-brain barrier (Dadparvar et al. (2011) Toxicology Letters 206:60-66). In other cases, the compound may pass through the blood-brain barrier, but not in sufficient concentrations to overcome degradative processes within brain tissues that render the compound inactive (Dadparvar et al. (2011) Toxicology Letters 206:60-66).

Prior methods for delivery drugs across the BBB involve three general categories: (1) liposome-based methods, where the therapeutic agent is encapsulated within the carrier; (2) synthetic polymer-based methods, where particles are created using synthetic polymers to achieve precisely-defined size characteristics; and (3) direct conjugation of a carrier to a drug, where the therapeutic agent is covalently bound to a carrier such as insulin. Liposomes are attractive for transporting drugs across the BBB because of their large carrying capacity. However, liposomes are generally too large to effectively cross the BBB, are inherently unstable, and their constituent lipids are gradually lost by absorption by lipid-binding proteins in the plasma. Synthetic polymers have run into difficulties having the drug carried across the cell only to be trapped in an endothelial cell or a lysosome, instead of the desired result of being ejected into the brain parenchyma.

Direct conjugation of pharmacological agents with the substances that can be transported across the BBB, such as insulin, has also been attempted. Insulin and insulin-like growth factors are known to cross the blood brain barrier by specialized facilitated diffusion systems. (Reinhardt et al. (1994) Endocrinology 135(5): 1753-1761). Specific transporters also exist for glucose and for large amino acids such as tryptophan. However, the specificity of the insulin transporter has proved to be too high to allow pharmacological agents covalently linked to insulin to cross into the brain. Similar results have been obtained with glucose and amino acid conjugates, whose uptake has been observed to obey the same general principles as other low-molecular weight substances, with only uncharged molecules below 700 Da achieving significant access to the brain.

The present invention also relates, in part, to enhancement of delivery of lipophilic active agents across the blood-brain barrier, particularly wherein the lipophilic active agent infused edible compositions produce greater concentrations of lipophilic active agents in subjects' central nervous system tissues as compared to control compositions. It was surprisingly found that the presently disclosed formulations achieved faster absorption, higher peak absorption, and higher overall quantities of lipophilic active agent (nicotine), on average, in the blood than concentration-matched controls. Furthermore, it was surprisingly found that the presently disclosed formulations achieved up to 5.6-times as much nicotine upon analysis of the rat brain tissue than was recovered with the matching control formulation.

B. Processes for Enhancing Delivery Across the Blood Brain Barrier

A process is provided for making an edible product infused with a lipophilic active agent with enhanced delivery across the blood brain barrier in a subject, comprising:
(a) providing a therapeutically effective amount of a lipophilic active agent;
(b) providing a bioavailability enhancing agent;
(c) providing an edible substrate;
(d) contacting the edible substrate with an oil comprising the lipophilic active agent and the bioavailability enhancing agent; and
(e) dehydrating the edible substrate;
   thereby producing the edible product infused with a lipophilic active agent with enhanced delivery across the blood brain barrier in the subject; wherein the bioavailability enhancing agent comprises an edible oil comprising long chain fatty acids and/or medium chain fatty acids that enhance the bioavailability of the lipophilic active agent and enhance delivery across the blood brain barrier in the subject.

In some embodiments, the edible product is selected from the group consisting of a pill, tablet, lozenge, mini lozenge, capsule, caplet, pouch, gum, spray, food, and combinations thereof.

In some embodiments, the edible substrate is selected from the group consisting of inulin, starch, modified starches, xanthan gum, carboxymethyl cellulose, methyl cellulose, hydroxypropylmethyl cellulose, konjac, chitosan, tragacanth, karaya, ghatti, larch, carageenan, alginate, chemically modified alginate, agar, guar, locust bean, psyllium, tara, gellan, curdlan, pullan, gum arabic, gelatin, pectin, and combinations thereof.

In some embodiments, the edible product further comprises a flavoring agent selected from the group consisting of vanilla, vanillin, ethyl vanillin, orange oil, fruit and berry type flavorants, Dramboui, bourbon, scotch, whiskey, spearmint, lavender, cinnamon, chai, cardamon, apium graveolents, clove, cascarilla, nutmeg, sandalwood, bergamot, geranium, honey essence, rose oil, lemon oil, Japanese mint, cassia, caraway, cognac, jasmin, chamomile, menthol, ylang ylang, sage, fennel, pimenta, ginger, anise, chai, coriander, coffee, peppermint, wintergreen, mint oils from a species of the genus Mentha, and combinations thereof.

In some embodiments, the edible product further comprises an additive selected from the group consisting of a non-nicotine alkaloid, a mineral, a vitamin, a dietary supplement, a dietary mineral, a nutraceutical, an energizing agent, a soothing agent, a coloring agent, an amino acid, a chemsthetic agent, an antioxidant, a food grade emulsifier, a pH modifier, a botanical, a teeth whitening agent, a therapeutic agent, a sweetener, a flavorant, and combinations thereof.

In some embodiments, the bioavailability of the lipophilic active agent in a subject is at least 2 times, 5 times, or 10 times greater than the bioavailability of the lipophilic active agent in the subject in the absence of the edible oil comprising long chain fatty acids and/or medium chain fatty acids. In some embodiments, the concentration of lipophilic active agent in central nervous system tissue of the subject is at least 1.5 times, 2 times, or 5 times greater than the concentration of lipophilic active agent in central nervous system tissue in the subject in the absence of the edible oil comprising long chain fatty acids and/or medium chain fatty acids.

In some embodiments, the edible oil comprising long chain fatty acids and/or medium chain fatty acids is substantially free of omega-6 fatty acids.

In some embodiments, the long chain fatty acids and/or medium chain fatty acids are selected from the group consisting of oleic acid, undecanoic acid, valeric acid, heptanoic acid, pelargonic acid, capric acid, lauric acid, and eicosapentaenoic acid.

In some embodiments, a process for making an edible product infused with a lipophilic active agent is provided, comprising:
(a) providing a therapeutically effective amount of a lipophilic active agent;
(b) providing a bioavailability enhancing agent, wherein the bioavailability enhancing agent comprises an edible oil comprising long chain fatty acids and/or medium chain fatty acids and enhances the bioavailability of the lipophilic active agent;
(c) providing an edible substrate;
(d) contacting the edible substrate with an oil comprising the lipophilic active agent and the
   bioavailability enhancing agent; and
(e) dehydrating the edible substrate;
   thereby producing the edible product infused with a lipophilic active agent.

In some embodiments, a beverage product infused with a lipophilic active agent is provided obtainable by the steps of:
(i) providing the edible product infused with a lipophilic active agent as described above, wherein the edible product infused with a lipophilic active agent is tea leaves, coffee beans, or cocoa powder infused with a lipophilic active agent; and
(ii) steeping the tea leaves, coffee beans, or cocoa powder infused with a lipophilic active agent in a liquid;
   thereby producing the beverage product infused with the lipophilic active agent. In some embodiments, the beverage product infused with a lipophilic active agent is obtainable by the steps of:
(i) providing the edible product infused with a lipophilic active agent as described above, wherein the edible product infused with a lipophilic active agent is tea leaves, coffee beans, or cocoa powder infused with a lipophilic active agent; and
(ii) steeping the tea leaves, coffee beans, or cocoa powder infused with a lipophilic active agent in a liquid;
   thereby producing the beverage product infused with the lipophilic active agent.

In other aspects, a process for making a food product infused with a lipophilic active agent is provided comprising the steps of: (i) contacting a food product with an oil comprising a lipophilic active agent and a bioavailability enhancing agent; and (ii) dehydrating the food product; thereby producing the food product infused with the lipophilic active agent; wherein the food product infused with the lipophilic active agent comprises a therapeutically effective amount of the lipophilic active agent, and further wherein: (a) the bioavailability enhancing agent enhances the bioavailability of the lipophilic active agent; and (b) the food product is selected from the group consisting of tea leaves, coffee beans, cocoa powder, meats, fish, fruits, vegetables, dairy products, legumes, pastas, breads, grains, seeds, nuts, spices, and herbs. In another aspect, step (i) comprises saturating the food product in the oil comprising the lipophilic active agent and the bioavailability enhancing agent. In another aspect, step (i) further comprises contacting the food product with a flavoring agent, particularly wherein the flavoring agent is selected from the group consisting of vanilla, vanillin, ethyl vanillin, orange oil, fruit and berry type flavorants, Dramboui, bourbon, scotch, whiskey, spearmint, lavender, cinnamon, chai, cardamon, apium graveolents, clove, cascarilla, nutmeg, sandalwood, bergamot, geranium, honey essence, rose oil, lemon oil, Japanese mint, cassia, caraway, cognac, jasmin, chamomile, menthol, ylang ylang, sage, fennel, pimenta, ginger, anise, chai, coriander, coffee, peppermint, wintergreen, mint oils from a species of the genus Mentha, and combinations thereof. In another aspect, the process further comprises a step of lyophilizing the food product infused with the lipophilic active agent.

In a further aspect, where the food product infused with the lipophilic active agent is tea leaves, coffee beans, or cocoa powder infused with the lipophilic active agent, the process further comprises packaging the tea leaves, coffee beans, or cocoa powder infused with the lipophilic active agent in single or multiple serve delivery devices, such as tea bags, water permeable membranes, pre-packaged beverage pods such as K-CUP® packs manufactured and sold by Keurig Inc. of Wakefield, Mass., and the like. Examples include, but are not limited to, such delivery devices and related systems as described in U.S. Pat. Nos. 3,450,024; 5,325,765; 5,840,189; and 6,606,938. In a particular aspect, the food product infused with the lipophilic active agent is tea leaves and the process further comprises packaging the tea leaves in tea bags.

In another aspect, a process for making a beverage product infused with a lipophilic active agent is provided comprising making tea leaves, coffee beans, or cocoa powder infused with the lipophilic active agent according to any of the processes described herein; further comprising the step of steeping the tea leaves, coffee beans, or cocoa powder infused with the lipophilic active agent in a liquid, thereby producing the beverage product infused with the lipophilic active agent.

In further aspects, the disclosed processes and methods use dehydration methods using dielectric energy, particularly microwave energy. In some aspects, the dielectric energy is selected from the group consisting of radio frequency energy, low frequency microwave energy, and high frequency microwave energy. In some aspects, the dehydration methods further comprise using dielectric energy under vacuum. In still further aspects, the dehydration methods further comprise stirring at a temperature of less than 70° C. In still further aspects, the disclosed processes and methods use dehydration methods using spray drying technology (e.g., methods of producing dry powders from a liquid or slurry by rapidly drying with a hot gas; see generally Mujumdar (2007) Handbook of Industrial Drying, CRC Press).

In some embodiments, the lipophilic active agent is selected from the group consisting of: cannabinoids, terpenes and terpenoids, non-steroidal anti-inflammatory drugs (NSAIDs), vitamins, nicotine or an analog thereof, phosphodiesterase 5 (PDE5) inhibitors, Maca extract, hormones, fentanyl or an analog thereof, buprenorphine or an analog thereof, scopolamine or an analog thereof, antioxidants, a nicotine compound, and an imaging agent.

C. Edible Substrates

The term “edible substrate” means any edible material, hard or soft, including varying degrees of hardness or softness. Examples of suitable substrates include, but are not limited to, inulin, starch, modified starches, xanthan gum, carboxymethyl cellulose, methyl cellulose, hydroxypropylmethyl cellulose, konjac, chitosan, tragacanth, karaya, ghatti, larch, carageenan, alginate, chemically modified alginate, agar, guar, locust bean, psyllium, tara, gellan, curdlan, pullan, gum arabic, gelatin, pectin, and combinations thereof.

Other suitable edible substrates include chewing gum, bubble gum, fat based gum, such as described in U.S. Patent Application Publication No. US 20080057155, incorporated herein by reference, candy gum, including crunch gum and marshmallow gum such as described in U.S. Patent Application Publication Nos. US 20080166449 and US 20080199564, each incorporated herein by reference, relatively soft/hard gums which turn hard/soft or remain soft/hard after chewing, candy, chocolate and combinations thereof including gum and candy combinations including soft and hard layers or regions with varying degrees of crunchiness, a layer or region of layering material as defined above, any other edible material that can be employed in an edible composition, including hard or soft layers or regions of conventional materials applied by conventional methods, such as hard panning and soft panning, or the like.

Additional edible substrates include gum base, sticky gum substrates, as well as hygroscopic, moisture sensitive and/or heat sensitive substrates.

D. Flavoring Agents and Additives

In some embodiments, the edible product further comprises a flavoring agent selected from the group consisting of vanilla, vanillin, ethyl vanillin, orange oil, fruit and berry type flavorants, Dramboui, bourbon, scotch, whiskey, spearmint, lavender, cinnamon, chai, cardamon, apium graveolents, clove, cascarilla, nutmeg, sandalwood, bergamot, geranium, honey essence, rose oil, lemon oil, Japanese mint, cassia, caraway, cognac, jasmin, chamomile, menthol, ylang ylang, sage, fennel, pimenta, ginger, anise, chai, coriander, coffee, peppermint, wintergreen, mint oils from a species of the genus Mentha, and combinations thereof.

In some embodiments, the edible product further comprises an additive selected from the group consisting of a non-nicotine alkaloid, a mineral, a vitamin, a dietary supplement, a dietary mineral, a nutraceutical, an energizing agent, a soothing agent, a coloring agent, an amino acid, a chemsthetic agent, an antioxidant, a food grade emulsifier, a pH modifier, a botanical, a teeth whitening agent, a therapeutic agent, a sweetener, a flavorant, and combinations thereof.

E. Bioavailability

In some embodiments, the bioavailability of the lipophilic active agent in a subject is at least 2 times, 5 times, or 10 times greater than the bioavailability of the lipophilic active agent in the subject in the absence of the edible oil comprising long chain fatty acids and/or medium chain fatty acids. In some embodiments, the edible oil comprising long chain fatty acids and/or medium chain fatty acids is substantially free of omega-6 fatty acids. In some embodiments, the long chain fatty acids and/or medium chain fatty acids are selected from the group consisting of oleic acid, undecanoic acid, valeric acid, heptanoic acid, pelargonic acid, capric acid, lauric acid, and eicosapentaenoic acid.

In some embodiments, the bioavailability enhancing agent is a protective colloid, an edible oil or fat, and a lipophilic active agent taste masking agent. In some embodiments, the bioavailability enhancing agent that is a protective colloid, an edible oil or fat, and a lipophilic active agent taste masking agent is nonfat dry milk.

Bioavailability refers to the extent and rate at which the active moiety (drug or metabolite) enters systemic circulation, thereby accessing the site of action. Bioavailability for a given formulation provides an estimate of the relative fraction of the orally administered dose that is absorbed into the systemic circulation. Low bioavailability is most common with oral dosage forms of poorly water-soluble, slowly absorbed drugs. Insufficient time for absorption in the gastrointestinal tract is a common cause of low bioavailability. If the drug does not dissolve readily or cannot penetrate the epithelial membrane (e.g., if it is highly ionized and polar), time at the absorption site may be insufficient. Orally administered drugs must pass through the intestinal wall and then the portal circulation to the liver, both of which are common sites of first-pass metabolism (metabolism that occurs before a drug reaches systemic circulation). Thus, many drugs may be metabolized before adequate plasma concentrations are reached.

Bioavailability is usually assessed by determining the area under the plasma concentration-time curve (AUC). AUC is directly proportional to the total amount of unchanged drug that reaches systemic circulation. Plasma drug concentration increases with extent of absorption; the maximum (peak) plasma concentration is reached when drug elimination rate equals absorption rate. Peak time is the most widely used general index of absorption rate; the slower the absorption, the later the peak time.

The bioavailability of some drugs is increased when co-administered with food, particularly agents such as cannabinoids that are Class II drugs under the Biopharmaceutical Drug Classification System (Kelepu et al. (2013) Acta Pharmaceutica Sinica B 3:361-372; Amidon et al. (1995) Pharm. Res. 12:413-420; Charman et al. (1997) J. Pharm. Sci. 86:269-282; Winstanley et al. (1989) Br. J. Clin. Pharmacol. 28:621-628). It is the lipid component of the food that plays a key role in the absorption of lipophilic drugs and that leads to enhanced oral bioavailability (Hunt & Knox (1968) J. Physiol. 194:327-336; Kelepu et al. (2013) Acta Pharmaceutica Sinica B 3:361-372). This has been attributed to the ability of a high fat meal to stimulate biliary and pancreatic secretions, to decrease metabolism and efflux activity, to increase intestinal wall permeability, and to a prolongation of gastrointestinal tract (GIT) residence time and transport via the lymphatic system (Wagnera et al. (2001) Adv. Drug Del. Rev. 50:513-31; Kelepu et al. (2013) Acta Pharmaceutica Sinica B 3:361-372). High fat meals also elevate triglyceride-rich lipoproteins that associate with drug molecules and enhance intestinal lymphatic transport, which leads to changes in drug disposition and changes the kinetics of the pharmacological actions of poorly soluble drugs (Gershkovich et al. (2007) Eur. J. Pharm. Sci. 32:24-32; Kelepu et al. (2013) Acta Pharmaceutica Sinica B 3:361-372). However, co-administration of food with lipophilic drugs requires close control and/or monitoring of food intake when dosing such drugs, and can also be subject to problems with patient compliance (Kelepu et al. (2013) Acta Pharmaceutica Sinica B 3:361-372).

In other aspects, the bioavailability enhancing agent within the compositions and methods of the present invention is an edible oil or fat, a protective colloid, or both a protective colloid and an edible oil or fat. In another aspect, the bioavailability enhancing agent is also a lipophilic active agent taste masking agent. In another particular aspect, where the bioavailability enhancing agent is both a protective colloid, an edible oil or fat, and a lipophilic active agent taste masking agent, the bioavailability enhancing agent is nonfat dry milk. In a further aspect, the bioavailability enhancing agent is substantially free of omega-6 fatty acids. In other aspects, the bioavailability of the lipophilic active agent in a subject is at least about 1.5 times, 2 times, 5 times, or 10 times greater than the bioavailability of the lipophilic active agent in the subject in the absence of the bioavailability enhancing agent. In a further aspect, the bioavailability of the lipophilic active agent in a subject is greater than 20%.

An edible oil is defined herein as an oil that is capable of undergoing de-esterification or hydrolysis in the presence of pancreatic lipase in vivo under normal physiological conditions. Specifically, digestible oils may be complete glycerol triesters of medium chain (C₇-C₁₃) or long chain (C₁₄-C₂₂) fatty acids with low molecular weight (up to C₆) mono-, di- or polyhydric alcohols. Some examples of digestible oils for use in this invention thus include: vegetable, nut, or seed oils (such as coconut oil, peanut oil, soybean oil, safflower seed oil, corn oil, olive oil, castor oil, cottonseed oil, arachis oil, sunflower seed oil, coconut oil, palm oil, rapeseed oil, evening primrose oil, grape seed oil, wheat germ oil, sesame oil, avocado oil, almond, borage, peppermint and apricot kernel oils) and animal oils (such as fish liver oil, shark oil and mink oil).

In a further aspect, the bioavailability enhancing agent is a long chain (C₁₄-C₂₂) fatty acid. In a further aspect, the bioavailability enhancing agent is a medium chain (C₇-C₁₃) fatty acid. In further aspects, the bioavailability enhancing agent is a combination of medium and long chain fatty acids.

Examples of protective colloids include polypeptides (such as gelatin, casein, and caseinate), polysaccharides (such as starch, dextrin, dextran, pectin, and gum arabic), as well as whole milk, skimmed milk, milk powder or mixtures of these. However, it is also possible to use polyvinyl alcohol, vinyl polymers, for example polyvinylpyrrolidone, (meth)acrylic acid polymers and copolymers, methylcellulose, carboxymethylcellulose, hydroxypropylcellulose and alginates. For further details, reference may be made to R. A. Morton, Fast Soluble Vitamins, Intern. Encyclopedia of Food and Nutrition, Vol. 9, Pergamon Press 1970, pages 128-131.

Oral administration constitutes the preferred route of administration for a majority of drugs. However, drugs that have an undesirable or bitter taste leads to lack of patient compliance in the case of orally administered dosage forms. In such cases, taste masking is an essential tool to improve patient compliance. Because lipophilic active agents (e.g., nicotine compounds) have an undesirable taste profile, in order to improve compliance, the presently disclosed compositions also comprise one or more lipophilic active agent taste masking agents. Examples of lipophilic active agent taste-masking agents include dry milk as described above, as well as menthol, sweeteners, sodium bicarbonate, ion-exchange resins, cyclodextrin inclusion compounds, adsorbates, and the like.

In another aspect, taste-masking agents used with the edible products infused with a lipophilic active agent of the present invention may further include flavoring agents such as salts (e.g., sodium chloride, potassium chloride, sodium citrate, potassium citrate, sodium acetate, potassium acetate, and the like), natural sweeteners (e.g., fructose, sucrose, glucose, maltose, mannose, galactose, lactose, and the like), artificial sweeteners (e.g., sucralose, saccharin, aspartame, acesulfame K, neotame, and the like); and mixtures thereof. In other aspects, suitable flavoring agents include, but are not limited to, vanilla, vanillin, ethyl vanillin, orange oil, fruit and berry type flavorants, Dramboui, bourbon, scotch, whiskey, spearmint, lavender, cinnamon, chai, cardamon, apium graveolents, clove, cascarilla, nutmeg, sandalwood, bergamot, geranium, honey essence, rose oil, lemon oil, Japanese mint, cassia, caraway, cognac, jasmin, chamomile, menthol, ylang ylang, sage, fennel, pimenta, ginger, anise, chai, coriander, coffee, peppermint, wintergreen, mint oils from a species of the genus Mentha, and combinations thereof.

In a further aspect, the bioavailability enhancing agent is substantially free of omega-6 fatty acids. As used herein, “substantially free” means largely but not wholly pure. For example, “substantially free” means less than 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, or 1.0% by weight.

In other aspects, the bioavailability of the lipophilic active agent in a subject is at least about 1.5 times, 2 times, 2.5 times, 3 times, 3.5 times, 4 times, 4.5 times, 5 times, 5.5 times, 6 times, 6.5 times, 7 times, 7.5 times, 8 times, 8.5 times, 9 times, 9.5 times, or 10 times greater than the bioavailability of the lipophilic active agent in the subject in the absence of the bioavailability enhancing agent.

In a further aspect, the bioavailability of the lipophilic active agent in a subject is greater than 20% or at least about 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or greater.

In other aspects, the concentration of the lipophilic active agent in central nervous system tissue in a subject is at least about 1.5 times, 2 times, 2.5 times, 3 times, 3.5 times, 4 times, 4.5 times, 5 times, 5.5 times, 6 times, 6.5 times, 7 times, 7.5 times, 8 times, 8.5 times, 9 times, 9.5 times, or 10 times greater than the concentration of the lipophilic active agent in central nervous system tissue in the subject in the absence of the bioavailability enhancing agent.

Assays and methods for measuring lipophilic active agent bioavailability are well known in the art (see, e.g., Rocci & Jusko (1983) Comput. Programs Biomed. 16:203-215; Shargel & Yu (1999) Applied biopharmaceutics & pharmacokinetics (4th ed.). New York: McGraw-Hill; Hu & Li (2011) Oral Bioavailability: Basic Principles, Advanced Concepts, and Applications, John Wiley & Sons Ltd.; Karschner et al. (2011) Clinical Chemistry 57:66-75; Ohlsson et al. (1980) Clin. Pharmacol. Ther. 28:409-416; Ohlsson et al. (1982) Biomed. Environ. Mass Spectrom. 9:6-10; Ohlsson et al. (1986) Biomed. Environ. Mass Spectrom. 13:77-83; Karschner et al. (2010) Anal. Bioanal. Chem. 397:603-611).

F. Lipophilic Active Agents

The active agents of the present invention are effective over a wide dosage range. For example, in treating adult humans, compositions and methods of the present invention comprise dosages of lipophilic active agents from 0.01 mg to 1,000 mg, from 0.5 mg to 500 mg, from 1 mg to 100 mg, from 5 mg to 50 mg, and from 10 mg to 25 mg. Alternatively, in treating adult humans, compositions and methods of the present invention comprise dosages of lipophilic active agents of 0.01 mg, 0.05 mg, 0.1 mg, 0.25 mg, 0.5 mg, 0.75 mg, 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1,000 mg.

In some embodiments, the lipophilic active agent is selected from the group consisting of: cannabinoids, terpenes and terpenoids, non-steroidal anti-inflammatory drugs (NSAIDs), vitamins, nicotine or an analog thereof, phosphodiesterase 5 (PDE5) inhibitors, Maca extract, hormones, fentanyl or an analog thereof, buprenorphine or an analog thereof, scopolamine or an analog thereof, antioxidants, a nicotine compound, and an imaging agent.

i. Cannabinoids

Cannabis sativa L. is one of the most widely used plants for both recreational and medicinal purposes. Over 500 natural constituents have been isolated and identified from C. sativa covering several chemical classes (Ahmed et al. (2008) J. Nat. Prod. 71:536-542; Ahmed et al. (2008) Tetrahedron Lett. 49:6050-6053; ElSohly & Slade (2005) Life Sci. 78:539-548; Radwan et al. (2009) J. Nat. Prod. 72:906-911; Radwan et al. (2008) Planta Medica. 74:267-272; Radwan et al. (2008) J. Nat. Prod. 69:2627-2633; Ross et al. (1995) ZagazigJ. Pharm. Sci. 4:1-10; Turner et al. (1980) J. Nat. Prod. 43:169-170). Cannabinoids belong to the chemical class of terpenophenolics, of which at least 85 have been uniquely identified in Cannabis (Borgelt et al. (2013) Pharmacotherapy 33:195-209).

Cannabinoids are ligands to cannabinoid receptors (CB₁, CB₂) found in the human body (Pertwee (1997) Pharmacol. Ther. 74:129-180). The cannabinoids are usually divided into the following groups: classical cannabinoids; non-classical cannabinoids; aminoalkylindole-derivatives; and eicosanoids (Pertwee (1997) Pharmacol. Ther. 74:129-180). Classical cannabinoids are those that have been isolated from C. sativa L. or their synthetic analogs. Non-classical cannabinoids are bi- or tri-cyclic analogs of tetrahydrocannabinol (THC) (without the pyran ring). Aminoalkylindoles and eicosanoids are substantially different in structure compared to classical and non-classical cannabinoids. The most common natural plant cannabinoids (phytocannabinoids) are cannabidiol (CBD), cannabigerol (CBG), cannabichromene (CBC), and cannabinol (CBN). The most psychoactive cannabinoid is Δ⁹-THC.

In recent years, marijuana and its components have been reported in scientific literature to counter the symptoms of a broad range of conditions including but not limited to multiple sclerosis and other forms of muscular spasm; movement disorders; pain, including migraine headache; glaucoma; asthma; inflammation; insomnia; and high blood pressure. There may also be utility for cannabinoids as anxiolytics, anti-convulsives, anti-depressants, anti-psychotics, anti-cancer agents, as well as appetite stimulants. Pharmacological and toxicological studies of cannabinoids have largely been focused on a synthetic analog of Δ⁹-THC (commercially available under the generic name Dronabinol). In 1985, Dronabinol was approved by the FDA for the treatment of chemotherapy associated nausea and vomiting, and later for AIDS-associated wasting and anorexia.

Therapeutic use of cannabinoids has been hampered by the psychoactive properties of some compounds (e.g., Dronabinol) as well as their low bioavailability when administered orally. Bioavailability refers to the extent and rate at which the active moiety (drug or metabolite) enters systemic circulation, thereby accessing the site of action. The low bioavailability of orally ingested cannabinoids (from about 6% to 20%; Adams & Martin (1996) Addiction 91: 1585-614; Agurell et al. (1986) Pharmacol. Rev. 38: 21-43; Grotenhermen (2003) Clin. Pharmacokinet. 42: 327-60) has been attributed to their poor dissolution properties and extensive first pass metabolism.

Cannabinoids are a heteromorphic group of chemicals which directly or indirectly activate the body's cannabinoid receptors. There are three main types of cannabinoids: herbal cannabinoids that occur uniquely in the Cannabis plant, synthetic cannabinoids that are manufactured, and endogenous cannabinoids that are produced in vivo. Herbal cannabinoids are nearly insoluble in water but soluble in lipids, alcohol, and non-polar organic solvents. These natural cannabinoids are concentrated in a viscous resin that is produced in glandular structures known as trichomes. In addition to cannabinoids, the resin is rich in terpenes, which are largely responsible for the odor of the Cannabis plant.

The identification of Δ⁹-tetrahydrocannabinol (THC) as a major psychoactive drug and its chemical synthesis in 1964 opened a new era of synthetic cannabinoids as pharmacological agents. Cannabinoid research has increased tremendously in recent years since the discovery of cannabinoid receptors and the endogenous ligands for these receptors. The receptors include CB1, predominantly expressed in the brain, and CB2, primarily found on the cells of the immune system. Cannabinoid receptors belong to a superfamily of G-protein-coupled receptors. They are single polypeptides with seven transmembrane α-helices, and have an extracellular, glycosylated N-terminus and intracellular C-terminus. Both CB1 and CB2 cannabinoid receptors are linked to G1/0-proteins. In addition to these receptors, endogenous ligands for these receptors capable of mimicking the pharmacological actions of THC have also been discovered. Such ligands were designated endocannabinoids and included anandamide and 2-arachidonoyl glycerol (2-AG). Anandamide is produced in the brain and peripheral immune tissues such as the spleen.

Unlike THC, which exerts its action by binding to CB1 and CB2, cannabidiol does not bind to these receptors and hence has no psychotropic activity. Instead, cannabidiol indirectly stimulates endogenous cannabinoid signaling by suppressing the enzyme that breaks down anandamide (fatty acid amide hydroxylase, “FAAH”). Cannabidiol also stimulates the release of 2-AG. Cannabidiol has been reported to have immunomodulating and anti-inflammatory properties, to exhibit anticonvulsive, anti-anxiety, and antipsychotic activity, and to function as an efficient neuroprotective antioxidant.

Cannabinoids in Cannabis are often inhaled via smoking, but may also be ingested. Smoked or inhaled cannabinoids have reported bioavailabilities ranging from 2-56%, with an average of about 30% (Huestis (2007) Chem. Biodivers. 4:1770-1804; McGilveray (2005) Pain Res. Manag. 10 Suppl. A:15A-22A). This variability is mainly due to differences in smoking dynamics. Cannabinoids that are absorbed through the mucous membranes in the mouth (buccomucosal application) have bioavailabilities of around 13% (Karschner et al. (2011) Clin. Chem. 57:66-75). By contrast, when cannabinoids are ingested, bioavailability is typically reduced to about 6% (Karschner et al. (2011) Clin. Chem. 57:66-75).

Accordingly, in other aspects, within the compositions and methods of the present invention, the lipophilic active agent is a cannabinoid.

In particular aspects, at least one cannabinoid within the compositions and methods of the present invention is selected from the group consisting of:

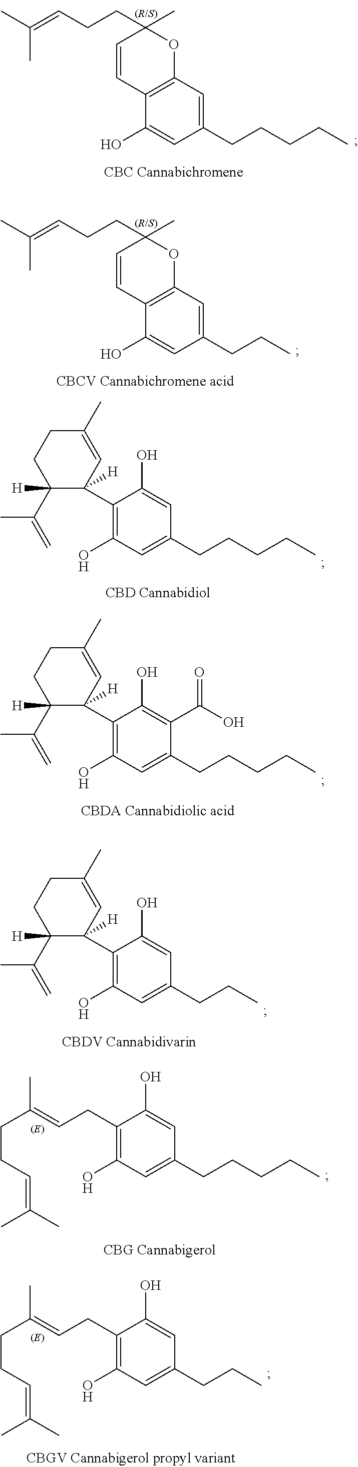

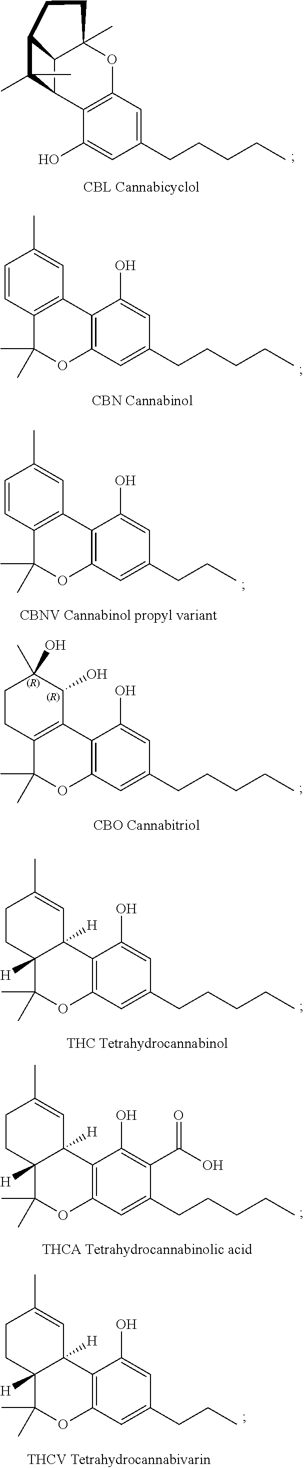

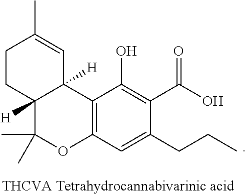 [SMILES: CCCc1cc2c(c(O)c1C(=O)O)[C@@H]1C=C(C)CC[C@H]1C(C)(C)O2]

In particular aspects, at least one cannabinoid within the compositions and methods of the present invention is a non-psychoactive cannabinoid such as cannabidiol. In some particularly disclosed aspects, the cannabinoid is selected from the group consisting of.

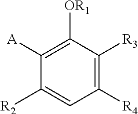

where A is aryl, and particularly

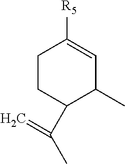

but not a pinene such as:

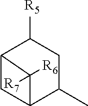

and the R₁-R₅ groups are each independently selected from the groups of hydrogen, lower substituted or unsubstituted alkyl, substituted or unsubstituted carboxyl, substituted or unsubstituted alkoxy, substituted or unsubstituted alcohol, and substituted or unsubstituted ethers, and R₆-R₇ are H or methyl. In particular aspects, there are no nitrogens in the rings, and/or no amino substitutions on the rings.

In other aspects, the cannabinoid is selected from the group consisting of:

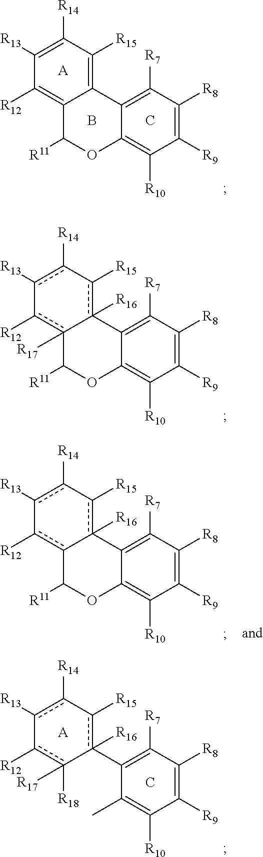

where there can be 0 to 3 double bonds on the A ring, as indicated by the optional double bonds indicated by dashed lines on the A ring. The C ring is aromatic, and the B ring can be a pyran. Particular aspects are dibenzo pyrans and cyclohexenyl benzenediols. Particular aspects of the cannabinoids of the present invention may also be highly lipid soluble, and in particular aspects can be dissolved in an aqueous solution only sparingly (for example 10 mg/ml or less). The octanol/water partition ratio at neutral pH in useful aspects is 5000 or greater, for example 6000 or greater. This high lipid solubility enhances penetration of the drug into the central nervous system (CNS), as reflected by its volume of distribution (V_{d}) of 1.5 L/kg or more, for example 3.5 L/kg, 7 L/kg, or ideally 10 L/kg or more, for example at least 20 L/kg. Particular aspects may also be highly water soluble derivatives that are able to penetrate the CNS, for example carboxyl derivatives.

R₇₋₁₈ are independently selected from the group of H, substituted or unsubstituted alkyl, especially lower alkyl, for example unsubstituted C₁-C₃ alkyl, hydroxyl, alkoxy, especially lower alkoxy such as methoxy or ethoxy, substituted or unsubstituted alcohol, and unsubstituted or substituted carboxyl, for example COOH or COCH₃. In other aspects R₇₋₁₈ can also be substituted or unsubstituted amino, and halogen.

In particular aspects, at least one cannabinoid within the compositions and methods of the present invention is a non-psychoactive cannabinoid, meaning that the cannabinoid has substantially no psychoactive activity mediated by the cannabinoid receptor (for example an IC₅₀ at the cannabinoid receptor of greater than or equal to 300 nM, for example greater than 1 μM and a Kᵢ greater than 250 nM, especially 500-1000 nM, for example greater than 1000 nM).

In other particular aspects, the cannabinoids within the compositions and methods of the present invention are selected from the group consisting of:

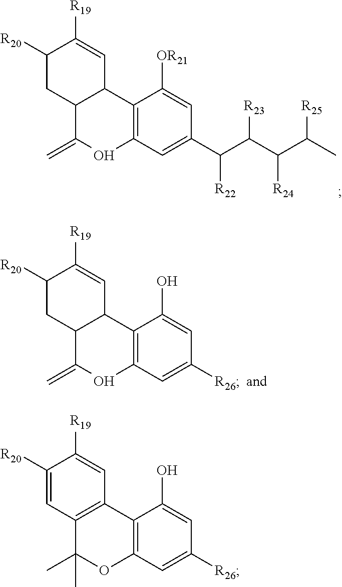

where R₁₉ is substituted or unsubstituted alkyl, such as lower alkyl (for example methyl), lower alcohol (such as methyl alcohol) or carboxyl (such as carboxylic acid) and oxygen (as in ═O); R₂₀ is hydrogen or hydroxy; R₂₁ is hydrogen, hydroxy, or methoxy; R₂₂ is hydrogen or hydroxy; R₂₃ is hydrogen or hydroxy; R₂₄ is hydrogen or hydroxy; R₂₅ is hydrogen or hydroxy; and R₂₆ is substituted or unsubstituted alkyl (for example n-methyl alkyl), substituted or unsubstituted alcohol, or substituted or unsubstituted carboxy.

In other particular aspects, the cannabinoids within the compositions and methods of the present invention are selected from the group consisting of:

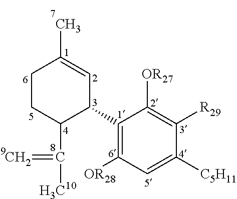

wherein numbering conventions for each of the ring positions are shown, and R₂₇, R₂₈ and R₂₉ are independently selected from the group consisting of H, unsubstituted lower alkyl such as CH₃, and carboxyl such as COCH₃. Particular examples of nonpsychoactive cannabinoids that fall within this definition are cannabidiol and

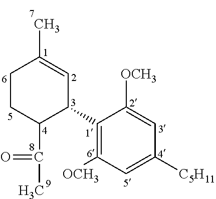

and other structural analogs of cannabidiol.

In other particular aspects, the cannabinoids within the compositions and methods of the present invention are selected from the group consisting of:

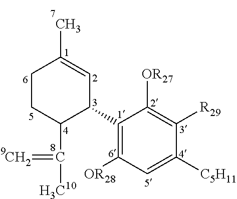

wherein R₂₇, R₂₈ and R₂₉ are independently selected from the group consisting of H, lower alkyl such as CH₃, and carboxyl such as COCH₃, and particularly wherein:
a) R₂₇=R₂₈=R₂₉═H
b) R₂₇=R₂₉═H; R₂₈═CH₃
c) R₂₇=R₂₈═CH₃; R₂₉═H
d) R₂₇=R₂₈═COCH₃; R₂₉═H
e) R₂₇═H; R₂₈=R₂₉═COCH₃
   When R₂₇=R₂₈=R₂₉═H, then the compound is cannabidiol (CBD). When R₂₇=R₂₉═H and R₂₈═CH₃, the compound is CBD monomethyl ether. When R₂₇=R₂₈═CH₃ and R₂₉═H, the compound is CBD dimethyl ether. When R₂₇=R₂₈═COCH₃ and R₂₉═H, the compound is CBD diacetate. When R₂₇═H and R₂₈=R₂₉═COCH₃, the compound is CBD monoacetate.

ii. Terpenes and Terpenoids

Terpenes are a diverse group of organic hydrocarbons derived from 5-carbon isoprene units and are produced by a wide variety of plants. Terpenoids are terpenes which have been chemically modified to add functional groups including heteroatoms. Terpenes and terpenoids are important building blocks for hormones, vitamins, pigments, steroids, resins, and essential oils. Terpenes are naturally present in Cannabis; however, they can be removed during the extraction process. Terpenes and terpenoids have various pharmaceutical (pharmacodynamic) effects and can be selected for the desired pharmaceutical activities.

In one embodiment, the terpene/terpenoid includes limonene. Limonene is a colorless liquid hydrocarbon classified as a cyclic terpene. The more common D-isomer possesses a strong smell of oranges and a bitter taste. It is used in chemical synthesis as a precursor to carvone and as a solvent in cleaning products. Limonene is a chiral molecule. Biological sources produce one enantiomer—the principal industrial source—citrus fruit, contains D-limonene ((+)-limonene), which is the (R)-enantiomer (CAS number 5989-27-5, EINECS number 227-813-5). Racemic limonene is known as dipentene. Its IUPAC name is 1-methyl-4-(1-methylethenyl)-cyclohexene. It is also known as 4-isopropenyl-1-methylcyclohexenep-Menth-1,8-dieneRacemic: DL-limonene; dipentene.

Limonene has a history of use in medicine, food and perfume. It has very low toxicity, and humans are rarely allergic to it. Limonene is used as a treatment for gastric reflux and as an anti-fungal agent. Its ability to permeate proteins makes it a useful treatment for toenail fungus. Limonene is also used for treating depression and anxiety. Limonene is reported to assist in the absorption of other terpenoids and chemicals through the skin, mucous membranes and digestive tract. Limonene has immunostimulant properties. Limonene is also used as botanical insecticide

The principle metabolites of limonene are (+)- and (−)-trans-carveol, a product of 6-hydroxylation) and (+)- and (−)-perillyl alcohol, a product of 7-hydroxylation by CYP2C9 and CYP2C19 cytochromes in human liver microsomes. The enantiomers of perillyl alcohol have been researched for possible pharmacological possibilities as dietary chemotherapeutic agents. They are considered novel therapeutic options in some CNS neoplasms and other solid tumors, especially for treatment of gliomas. The cytotoxic activities of perillyl alcohol and limonene metabolites are likely due to their antiangiogenic properties, hyperthermia inducing effects, negative apoptosis regulation and effect on Ras pathways.

In another embodiment, the terpene/terpenoid includes linalool. Linalool is a naturally occurring terpene alcohol chemical found in many flowers and spice plants with many commercial applications, the majority of which are based on its pleasant scent (floral and slightly spicy). It is also known as β-linalool, linalyl alcohol, linaloyl oxide, p-linalool, allo-ocimenol, and 3,7-dimethyl-1,6-octadien-3-ol. Its IUPAC name is 3,7-dimethylocta-1,6-dien-3-ol.

More than 200 species of plants produce linalool, mainly in the families Lamiaceae, Lauraceae and Rutaceae. It has also been found in some fungi. Linalool has been used for thousands of years as a sleep aid. Linalool is an important precursor in the formation of Vitamin E. It has a history of use in the treatment of both psychosis and anxiety, and as an anti-epileptic agent. It also provides analgesic pain relief. Its vapors have been shown to be an effective insecticide against fleas, fruit flies and cockroaches. Linalool is used as a scent in an estimated 60-80% of perfumed hygiene products and cleaning agents including soaps, detergents, shampoos and lotions.

In another embodiment, the terpene/terpenoid includes myrcene. Myrcene, or 0-myrcene, is an olefinic natural organic compound. It is classified as a hydrocarbon, more precisely as a monoterpene. Terpenes are dimers of isoprene, and myrcene is one of the most important. Myrcene is a component of the essential oil of several plants including bay, Cannabis, ylang-ylang, wild thyme, mango, parsley and hops. Myrcene is produced mainly semi-synthetically from myrcia, from which it gets its name. Myrcene is a key intermediate in the production of several fragrances. α-Myrcene is the name for the structural isomer 2-methyl-6-methylene-1,7-octadiene, which is not found in nature and is little used. Its IUPAC name is 7-methyl-3-methylene-1,6-octadiene.

Myrcene has an analgesic effect and is likely to be responsible for the medicinal properties of lemon grass tea. It has anti-inflammatory properties through Prostaglandin E2. The analgesic action can be blocked by naloxone or yohimbine in mice, which suggests mediation by alpha 2-adrenoceptor stimulated release of endogenous opioids. β-Myrcene is reported to have anti-inflammatory properties, and is used to treat spasms, sleep disorders and pain. Myrcene appears to lower resistance across the blood to brain barrier, allowing itself and many other chemicals to cross the barrier more effectively.

In another embodiment, the terpene/terpenoid includes α-Pinene. α-Pinene is one of the primary monoterpenes that is physiologically critical in both plants and animals. It is an alkene and it contains a reactive four-membered ring. α-Pinene tends to react with other chemicals, forming a variety of other terpenes including D-limonene and other compounds. α-Pinene has been used for centuries as a bronchodilator in the treatment of asthma. It is highly bioavailable with 60% human pulmonary uptake with rapid metabolism. α-Pinene is an anti-inflammatory via PGE1, and appears to be a broad-spectrum antibiotic. It acts as an acetylcholinesterase inhibitor, aiding memory. Products of α-pinene which have been identified include pinonaldehyde, norpinonaldehyde, pinic acid, pinonic acid, and pinalic acid.

Pinene is found in conifer, pine and orange. α-Pinene is a major constituent in turpentine. Its IUPAC name is (1S,5S)-2,6,6-Trimethylbicyclo[3.1.1]hept-2-ene ((−)-α-Pinene).

In another embodiment, the terpene/terpenoid includes β-Pinene. β-Pinene is one of the most abundant compounds released by trees. It is one of the two isomers of pinene, the other being α-pinene. It is a common monoterpene, and if oxidized in air, the allylic products of the pinocarveol and myrtenol family prevail. Its IUPAC name is 6,6-dimethyl-2-methylenebicyclo[3.1.1]heptane and is also known as 2(10)-Pinene; Nopinene; Pseudopinene. It is found in cumin, lemon, pine and other plants.

In another embodiment, the terpene/terpenoid includes caryophyllene, also known as 3-caryophyllene. Caryophyllene is a natural bicyclic sesquiterpene that is a constituent of many essential oils, including clove, Cannabis, rosemary and hops. It is usually found as a mixture with isocaryophyllene (the cis double bond isomer) and α-humulene, a ring-opened isomer. Caryophyllene is notable for having a rare cyclobutane ring. Its IUPAC name is 4,11,11-trimethyl-8-methylene-bicyclo[7.2.0]undec-4-ene.

Caryophyllene is known to be one of the compounds that contribute to the spiciness of black pepper. In a study conducted by the Swiss Federal Institute of Technology, β-caryophyllene was shown to be selective agonist of cannabinoid receptor type-2 (CB2) and to exert significant cannabimimetic, anti-inflammatory effects in mice. Anti-nociceptive, neuroprotective, anxiolytic, antidepressant and anti-alcoholic activity have been tied to caryophyllene. Because β-caryophyllene is an FDA approved food additive, it is considered the first dietary cannabinoid.

In another embodiment, the terpene/terpenoid includes citral. Citral, or 3,7-dimethyl-2,6-octadienal or lemonal, is either a pair, or a mixture of terpenoids with the molecular formula C₁₀H₁₆O. The two compounds are double bond isomers. The E-isomer is known as geranial or citral A. The Z-isomer is known as neral or citral B. Its IUPAC name is 3,7-dimethylocta-2,6-dienal. It is also known as citral, geranial, neral, geranialdehyde.

Citral is present in the oils of several plants, including lemon myrtle, lemongrass, verbena, lime, lemon and orange. Geranial has a pronounced lemon odor. Neral's lemon odor is not as intense, but sweet. Citral is primarily used in perfumery for its citrus quality. Citral is also used as a flavor and for fortifying lemon oil. It has strong antimicrobial qualities, and pheromonal effects in insects. Citral is used in the synthesis of vitamin A, ionone and methylionone.

In another embodiment, the terpene/terpenoid includes humulene. Humulene, also known as α-humulene or α-caryophyllene, is a naturally occurring monocyclic sesquiterpene (C₁₅H₂₄), which is an 11-membered ring consisting of 3 isoprene units containing three nonconjugated C═C double bonds, two of them being triply substituted and one being doubly substituted. It was first found in the essential oils of Humulus lupulus (hops). Humulene is an isomer of β-caryophyllene, and the two are often found together as a mixture in many aromatic plants.

Humulene has been shown to produce anti-inflammatory effects in mammals, which demonstrates potential for management of inflammatory diseases. It produces similar effects to dexamethasone, and was found to decrease the edema formation caused by histamine injections. Humulene produced inhibitory effects on tumor necrosis factor-α (TNFα) and interleukin-1.beta. (IL1B) generation in carrageenan-injected rats. In Chinese medicine, it is blended with β-caryophyllene and used as a remedy for inflammation.

Other exemplary terpenes and terpenoids include menthol, eucalyptol, borneol, pulegone, sabinene, terpineol, and thymol. In one embodiment, an exemplary terpene/terpenoid is eucalyptol.

iii. NSAIDs

NSAIDs are the second-largest category of pain management treatment options in the world. The global pain management market was estimated at $22 billion in 2011, with $5.4 billion of this market being served by NSAID's. The U.S. makes up over one-half of the global market. The opioids market (such as morphine) form the largest single pain management sector but are known to be associated with serious dependence and tolerance issues.

Although NSAIDs are generally a safe and effective treatment method for pain, they have been associated with a number of gastrointestinal problems including dyspepsia and gastric bleeding.

Delivery of NSAIDs through the compositions and methods of the present invention will provide the beneficial properties of pain relief with lessened negative gastrointestinal effects, and also deliver lower dosages of active ingredients in order to provide pain management outcomes across a variety of indications.

Accordingly, in other aspects, within the compositions and methods of the present invention, the lipophilic active agent is an NSAID, particularly wherein the NSAID is selected from the group consisting of acetylsalicylic acid, ibuprophen, acetaminophen, diclofenac, indomethacin, and piroxicam.

In some aspects, the NSAID is a COX inhibitor, e.g., a selective COX inhibitor, e.g., a COX-2 inhibitor, e.g., celecoxib, deracoxib, valdecoxib, rofecoxib, tilmacoxib, or other similar known compounds, especially celecoxib, including its various known crystalline forms and various salts thereof (e.g., crystalline forms I, II, III, IV and N). In some aspects, active agents within the compositions according to the present invention are selective COX-2 inhibitors, which are known to be useful for treating: inflammation, colorectal polyps (because they have effects on abnormally dividing cells such as those of precancerous colorectal polyps), menstrual cramps, sports injuries, osteoarthritis, rheumatoid arthritis, and pain, e.g., acute pain, and for reducing the risk of peptic ulceration. Aspects of the invention are suitable for use with crystalline or amorphous forms of active ingredients.

In one aspect, the active agent is celecoxib, which is a selective COX-2 inhibitor having about 7.6-times higher affinity towards COX-2 than towards COX-1. Thus the anti-inflammatory activity of celecoxib is only rarely accompanied with gastrointestinal side effects which are often experienced with non-selective non-steroidal anti-inflammatory active ingredients.

iv. Vitamins

The global vitamin and supplement market is worth $68 billion according to Euromonitor. The category is both broad and deep, comprised of many popular and some lesser known substances. Vitamins in general are thought to be an $8.5 billion annual market in the U.S. The U.S. is the largest single national market in the world, and China and Japan are the 2^{nd} and 3^{rd} largest vitamin markets.

The four most common fat-soluble vitamins are: vitamin A (retinol), vitamin D (calciferol), vitamin E (tocopherol), and vitamin K (phylloquinone and menaquinone).

Vitamin E is fat soluble and can be incorporated into cell membranes which can protect them from oxidative damage. Global consumption of natural source vitamin E was 10,900 metric tons in 2013 worth $611.9 million.

Accordingly, in other aspects, within the compositions and methods of the present invention, the lipophilic active agent is a fat soluble vitamin, particularly wherein the fat soluble vitamin is vitamin A, D, E, or K.

v. Nicotine Compounds

Nicotine is a natural ingredient in tobacco leaves where it acts as a botanical insecticide (Hukkanen et al. (2005) Pharmacological Reviews 57:79-115). Comprising about 95% of the total alkaloid content of commercial cigarette tobacco, nicotine comprises about 1.5% by weight of commercial cigarette tobacco (Hukkanen et al. (2005) Pharmacological Reviews 57:79-115). Although oral snuff and pipe tobacco contain concentrations of nicotine similar to cigarette tobacco, cigar and chewing tobacco typically contain only about half of the nicotine concentration of cigarette tobacco (Hukkanen et al. (2005) Pharmacological Reviews 57:79-115). An average tobacco rod typically contains 10 to 14 mg of nicotine (Hukkanen et al. (2005) Pharmacological Reviews 57:79-115), and on average about 1 to 1.5 mg of nicotine is absorbed systemically during smoking (Hukkanen et al. (2005) Pharmacological Reviews 57:79-115). The nicotine in tobacco is largely the levorotary (S)-isomer, only 0.1 to 0.6% of total nicotine content is (R)-nicotine (Hukkanen et al. (2005) Pharmacological Reviews 57:79-115). The (R)-nicotine content of tobacco smoke is higher, with up to 10% of nicotine in smoke reported to be (R)-isomer, and thought to be attributed to racemization occurring during combustion (Hukkanen et al. (2005) Pharmacological Reviews 57:79-115).

More than 99% of all nicotine that is consumed worldwide is delivered through smoking cigarettes. Approximately 6,000,000 deaths per year, worldwide, are attributed primarily to the delivery of nicotine through the act of smoking according to the Centers for Disease Control and Prevention, which also estimates that over $170 billion per year is spent just in the U.S. on direct medical care costs for adult smokers. In any twelve month period, 69% of U.S. adult smokers want to quit smoking and 43% of U.S. adult smokers have attempted to quit.

Worldwide, retail cigarette sales were worth $722 billion in 2013, with over 5.7 trillion cigarettes sold to more than 1 billion smokers. It would be desirable in the art to provide further methods for altering the character and nature of tobacco (and tobacco compositions and formulations) useful in smoking articles and/or or smokeless tobacco products, including enhancement of bioavailability of active agents, masking of unpleasant tastes, and the incorporation of additional active agents. Furthermore, the delivery of nicotine to satisfy current demand via the compositions and methods of the present invention, can in part alleviate the consumer demand for cigarettes. Since most of the adverse health outcomes of nicotine consumption are associated with the delivery method and only to a lesser degree to the actual ingestion of nicotine, a vast positive community health outcome can be achieved through the reduction in smoking cigarettes.

Accordingly, in other aspects, within the compositions and methods of the present invention, the lipophilic active agent is a nicotine compound.

As used herein, “nicotine compound” or “source of nicotine” often refers to naturally-occurring or synthetic nicotine compound unbound from a plant material, meaning the compound is at least partially purified and not contained within a plant structure, such as a tobacco leaf Most preferably, nicotine is naturally-occurring and obtained as an extract from a Nicotiana species (e.g., tobacco). The nicotine can have the enantiomeric form S(−)-nicotine, R(+)-nicotine, or a mixture of S(−)-nicotine and R(+)-nicotine. Most preferably, the nicotine is in the form of S(−)-nicotine (e.g., in a form that is virtually all S(−)-nicotine) or a racemic mixture composed primarily or predominantly of S(−)-nicotine (e.g., a mixture composed of about 95 weight parts S(−)-nicotine and about 5 weight parts R(+)-nicotine). Most preferably, the nicotine is employed in virtually pure form or in an essentially pure form. Highly preferred nicotine that is employed has a purity of greater than about 95 percent, more preferably greater than about 98 percent, and most preferably greater than about 99 percent, on a weight basis. Despite the fact that nicotine can be extracted from Nicotiana species, it is highly preferred that the nicotine (and the composition and products produced in accordance with the present invention) are virtually or essentially absent of other components obtained from or derived from tobacco.

Nicotine compounds can include nicotine in free base form, salt form, as a complex, or as a solvate. See, for example, the discussion of nicotine in free base form in US Pat. Pub. No. 2004/0191322 to Hansson, which is incorporated herein by reference. At least a portion of the nicotine compound can be employed in the form of a resin complex of nicotine, where nicotine is bound in an ion exchange resin, such as nicotine polacrilex. See, for example, U.S. Pat. No. 3,901,248 to Lichtneckert et al., which is incorporated herein by reference. At least a portion of the nicotine can be employed in the form of a salt. Salts of nicotine can be provided using the types of ingredients and techniques set forth in U.S. Pat. No. 2,033,909 to Cox et al. and U.S. Pat. No. 4,830,028 to Lawson et al., and Perfetti, Beitrage Tabakforschung Int., 12: 43-54 (1983), which are incorporated herein by reference. See, also, U.S. patent application Ser. No. 12/769,335 to Brinkley et al., filed Apr. 28, 2010, which is incorporated herein by reference. Additionally, salts of nicotine have been available from sources such as Pfaltz and Bauer, Inc. and K&K Laboratories, Division of ICN Biochemicals, Inc.

Exemplary pharmaceutically acceptable nicotine salts include nicotine salts of tartrate (e.g., nicotine tartrate and nicotine bitartrate) chloride (e.g., nicotine hydrochloride and nicotine dihydrochloride), sulfate, perchlorate, ascorbate, fumarate, citrate, malate, lactate, aspartate, salicylate, tosylate, succinate, pyruvate, and the like; nicotine salt hydrates (e.g., nicotine zinc chloride monohydrate), and the like. Additional organic acids that can form salts with nicotine include formic, acetic, propionic, isobutyric, butyric, alpha-methylbutyric, isovaleric, beta-methylvaleric, caproic, 2-furoic, phenylacetic, heptanoic, octanoic, nonanoic, oxalic, malonic, and glycolic acid, as well as other fatty acids having carbon chains of up to about 20 carbon atoms.

In many embodiments, the nicotine compound will be present in multiple forms. For example, the nicotine can be employed within the composition as a mixture of at least two salts (e.g., two different organic acid salts, such as a mixture of nicotine bitartrate and nicotine levulinate), as at least two salts that are segregated within the composition, in a free base form and salt form, in a free base form and a salt form that are segregated within the composition, in a salt form and in a complexed form (e.g., a resin complex such as nicotine polacrilex), in a salt for and in a complexed form that are segregated with in the composition, in a free base form and a complexed form, in a free base form and a complexed form that are segregated within the composition, or the like. As such, each single dosage unit or piece (e.g., gum piece, lozenge, sachet, film strip, etc.) can incorporate at least two forms of nicotine.

A nicotine compound, in particular a compound such as nicotine, also can be employed in combination with other so-called tobacco alkaloids (i.e., alkaloids that have been identified as naturally occurring in tobacco). For example, nicotine, as employed in accordance with the present invention, can be employed in combination with nornicotine, anatabine, anabasine, and the like, and combinations thereof. See, for example, Jacob et al., Am. J. Pub. Health, 5: 731-736 (1999), which is incorporated herein by reference.

The compositions of the invention most preferably possess a form that is pharmaceutically effective and pharmaceutically acceptable. That is, the composition most preferably does not incorporate to any appreciable degree, or does not purposefully incorporate, significant amounts of components of tobacco, other than nicotine. As such, pharmaceutically effective and pharmaceutically acceptable compositions do not include tobacco in parts or pieces, processed tobacco components, or many of the components of tobacco traditionally present within tobacco-containing cigarettes, cigars, pipes, or smokeless forms of tobacco products. Highly preferred compositions that are derived by extracting naturally-occurring nicotine from tobacco include less than 5 weight percent of tobacco components other than nicotine, more often less than about 0.5 weight percent, frequently less than about 0.25 weight percent, and typically are entirely absent or devoid of components of tobacco, processed tobacco components, or components derived from tobacco, other than nicotine, based on the total weight of the composition.

In some embodiments, the nicotine compound is selected from the group consisting of nicotine and a nicotine derivative, wherein the nicotine derivative comprises a nicotine salt, a nicotine complex, a nicotine polacrilex, or combinations thereof.

Tobacco alkaloids include nicotine and nicotine-like or related pharmacologically active compounds such as nor-nicotine, lobeline and the like, as well as the free base substance nicotine and all pharmacologically acceptable salts of nicotine, including acid addition salts. “Nicotine compounds” as that term is used herein therefore includes all the foregoing tobacco alkaloids, as well as nicotine salts including but not limited to nicotine hydrogen tartrate and nicotine bitartrate dihydrate, as well as nicotine hydrochloride, nicotine dihydrochloride, nicotine sulfate, nicotine citrate, nicotine zinc chloride monohydrate, nicotine salicylate, nicotine oil, nicotine complexed with cyclodextrin, polymer resins such as nicotine polacrilex, nicotine resinate, and other nicotine-ion exchange resins, either alone or in combination.

The nicotine compounds also include nicotine analogs that include, but are not limited to the structures shown below for (s)-Nicotine, Nornicotine, (S)-Cotinine, B-Nicotyrine, (S)-Nicotene-N′-Oxide, Anabasine, Anatabine, Myosmine, B-Nornicotyrine, 4-(Methylamino)-1-(3-pyridyl)-1-butene (Metanicotine) cis or trans, N′-Methylanabasine, N′Methylanatabine, N′Methylmyosmine, 4-(Methylamino)-1-(3-pyridyl)-1-butanone (Pseudoxynicotine), and 2,3′-Bipyridyl (Hukkanen et al. (2005) Pharmacological Reviews 57:79-115):

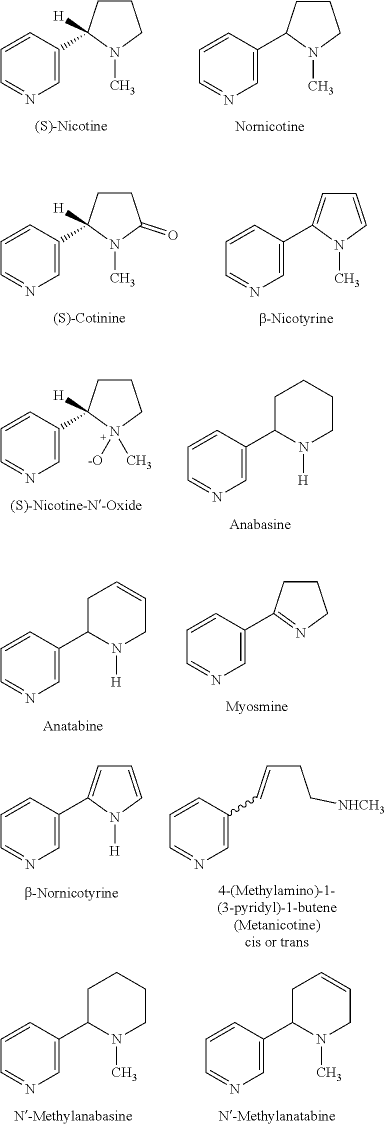

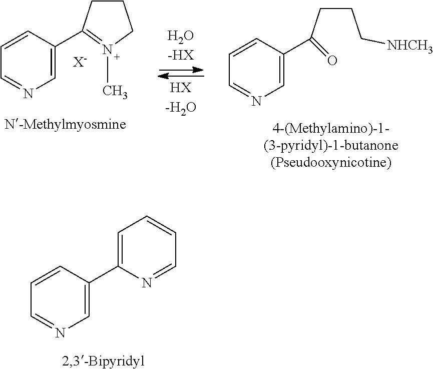

Nicotine compounds also include nicotine bitartrate, cytisine, nicotine polacrilex, nornicotine, nicotine 1-N-oxide, metanicotine, nicotine imine, nicotine N-glucuronide, N-methylnicotinium, N-n-decylnicotinium, 5′-cyanonicotine, 3,4-dihydrometanicotine, N′-methylnicotinium, N-octanoylnornicotine, 2,3,3a,4,5,9b-hexahydro-1-methyl-1H-pyrrolo(3,2-h)isoquinoline, 5-isothiocyanonicotine, 5-iodonicotine, 5′-hydroxycotinine-N-oxide, homoazanicotine, nicotine monomethiodide, N-4-azido-2-nitrophenylnornicotine, N-methylnornicotinium, nicotinium molybdophosphate resin, N-methyl-N′-oxonicotinium, N′-propylnornicotine, pseudooxynicotine, 4′-methylnicotine, 5-fluoronicotine, K(s-nic)5(Ga2(N,N′-bis-(2,3-dihydroxybenzoyI)-1,4-phenylenediamine)3), 5-methoxynicotine, 1-benzyl-4-phenylnicotinamidinium, 6-n-propylnicotine, SIB1663, 6-hydroxynicotine, N-methyl-nicotine, 6-(2-phenylethyl)nicotine, N′-formylnornicotine, N-n-octylnicotinium, N-(n-oct-3-enyl)nicotinium, N-(n-dec-9-enyl)nicotinium, 5‘-acetoxy-N’-nitrosonornicotine, 4-hydroxynicotine, 4-(dimethylphenylsilyl)nicotine, N′-carbomethoxynornicotine, and N-methylnicoton.

The nicotine compound may be used in one or more distinct physical forms well known in the art, including free base forms, encapsulated forms, ionized forms and spray-dried forms.

Additional description regarding the chemistry, absorption, metabolism, kinetics and biomarkers of nicotine is described in Hukkanen et al. (2005) Pharmacological Reviews 57:79-115 and Benowitz et al. (2009) Handb. Exp. Pharmacol. 192:29-60, which are both incorporated herein in their entireties.

The compositions also include nicotine compounds characterized as selective agonists to nicotinic receptor subtypes that are present in the brain, or that can otherwise be characterized as a compound that modulates nicotinic receptor subtypes of the CNS. Various nicotinic receptor subtypes are described in Dwoskin et al., Exp. Opin. Ther. Patents, 10: 1561-1581 (2000); Huang et al., J. Am. Chem. Soc., 127: 14401-14414 (2006); and Millar, Biochem. Pharmacol., 78: 766-776 (2009); which are incorporated herein by reference. Representative compounds that can be characterized as other nicotine compounds for purposes of this invention are set forth in Schmitt et al., Annual Reports in Med. Chem. 35: 41-51 (2000); and Arneric et al., Biochem. Pharmacol., 74: 1092-1101 (2007); which are incorporated herein by reference.

In one aspect, the nicotine compound can be a compound has selectivity to the α₇ (alpha 7) nicotinic receptor subtype, and preferably is an agonist of the W nicotinic receptor subtype. Several compounds having such W receptor subtype selectivity have been reported in the literature. For example, various compounds purported to have selectivity to the W nicotinic receptor subtype are set forth in Malysz et al., Assay Drug Dev. Tech., August: 374-390 (2009). An example of one such nicotine compound is N-[(2S,3S)-2-(pyridin-3-ylmethyl)-1-azabicyclo[2.2.2]oct-3-yl]-1-benzofur-an-2-carboxamide (also known as TC-5619). See, for example, Hauser et al., Biochem. Pharmacol., 78: 803-812 (2009). Another representative is compound is (5aS,8S,10aR)-5a,6,9,10-Tetrahydro, 7H,11H-8,10a-methanopyrido [2′,3′:5,6]pyrano[2,3-d]azepine (also known as dianicline or SSR591813 or SSR-591,813). See, for example, Hajos et al., J. Pharmacol. Exp. Ther., 312: 1213-1222 (2005). Another representative compound is 1,4-Diazabicyclo[3.2.2]nonane-4-carboxylic acid, 4-bromophenyl ester (also known as SSR180711). See, for example, Biton et al., Neuropsychopharmacol., 32: 1-16 (2007). Another representative compound is 3-[(3E)-3-[(2,4-dimethoxyphenyl)methylidene]-5,6-dihydro-4H-pyridin-2-yl]pyridine (also known as GTS-21). See, for example, U.S. Pat. No. 5,516,802 to Zoltewicz et al. and U.S. Pat. No. 5,741,802 to Kem et al. Another representative compound is 2-methyl-5-(6-phenyl-pyridazin-3-yl)-octahydro-pyrrolo[3,4-c]pyrrole (also known as A-582941). See, for example, Thomsen et al., Neuroscience, 154: 741-753 (2008). Another representative compound is (5S)-spiro[1,3-oxazolidine-5,8′-1-azabicyclo[2.2.2]octane]-2-one (also known as AR-R-17779 or AR-R-17779). See, for example, Li et al., Neuropsycopharmacol., 33: 2820-2830 (2008). Another representative compound is N-[(3R)-1-azabicyclo[2.2.2]oct-3-yl]-4-chlorobenzamide (also known as PNU-282,987). See, for example, Siok et al., Eur. J. Neurosci., 23: 570-574 (2006). Another representative compound is 5-morpholin-4-yl-pentanoic acid (4-pyridin-3-yl-phenyl)-amide (also known as WAY-317,538 or SEN-12333). See, for example, Roncarati et al., J. Pharmacol. Exp. Ther., 329: 459-468(2009). Yet other examples are compounds are those designated as EVP-6124 and EVP-4473 by Envivo Pharmaceuticals, Inc., TC-6987 by Targacept, Inc. and MEM3454 by Memory Pharmaceuticals Corp. The foregoing cited references are incorporated herein by reference.

In one aspect, the nicotine compound can be a compound that has selectivity to the α₄β₂ (alpha 4 beta 2) nicotinic receptor subtype, and preferably is an agonist of the α₄β₂ nicotinic receptor subtype. Several compounds having such α₄β₂ receptor subtype selectivity have been reported in the literature. An example of one such nicotine compound is known as 7,8,9,10-tetrahydro-6,10-methano-6H-pyrazino(2,3-h)(3) benzazepine (also known as varenicline and in the form of varenicline tartrate which is the active ingredient of a product commercially marketed under the tradename Chantix or Champix by Pfizer). See, for example, Jorenby et al., JAMA, 296: 56-63 (2006) and US Pat. Pub. No. 2010/0004451 to Ahmed et al. Another representative compound is (2S,4E)-5-(5-isopropoxypyridin-3-yl)-N-methylpent-4-en-2-amine (also known as ispronicline or AZD-3480 of AstraZeneca or TC-1734 of Targacept, Inc. (Winston-Salem, N.C., USA)). See, for example, Dunbar et al., Psychopharmacol. (Berlin), 191: 919-929 (2007). Another representative compound is [3-(2(S))-azetidinylmethoxy)pyridine] dihydrochloride, (also known as A-85380). See, for example, Schreiber, Psychopharmacol., 159:248-257 (2002). Another representative compound is (5aS,8S,10aR)-5a,6,9,10-Tetrahydro,7H,11H-8,10a-methanopyrido [2′,3′:5,6]pyrano[2,3-d]azepine (also known as SSR591813). See, for example, Cohen et al., Neuroscience, Pres. No. 811.5 (2002); and Cohen et al., J. Pharmacol. Exp. Ther., 306: 407-420 (2003). Another representative compound is known as A-969933. See, for example, Zhu et al., Biochem. Pharmacol., 78: 920 (2009). Other representative compounds are known as S35836-1 and S35678-1. See, for example, Lockhart et al., Neuroscience, Pres. No. 684.9 (2002). Yet other examples are compounds are those designated as 3-(5,6-Dichloro-pyridin-3-yl)-1S,5S-3,6-diazabicyclo[3.2.0]heptane (also known as Sofinicline or ABT-894) by Abbott Laboratories; AZD1446 by AstraZeneca and TC-6499 by Targacept, Inc. The foregoing cited references are incorporated herein by reference.

In some cases, the nicotine can be liquid nicotine. Liquid nicotine can be purchased from commercial sources, whether tobacco-derived or synthetic. Tobacco-derived nicotine can include one or more other tobacco organoleptic components other than nicotine. The tobacco-derived nicotine can be extracted from raw (e.g., green leaf) tobacco and/or processed tobacco. Processed tobaccos can include fermented and unfermented tobaccos, dark air-cured, dark fire cured, burley, flue cured, and cigar filler or wrapper, as well as the products from the whole leaf stemming operation. The tobacco can also be conditioned by heating, sweating and/or pasteurizing steps as described in U.S. Publication Nos. 2004/0118422 or 2005/0178398. Fermenting typically is characterized by high initial moisture content, heat generation, and a 10 to 20% loss of dry weight. See, e.g., U.S. Pat. Nos. 4,528,993; 4,660,577; 4,848,373; and 5,372,149. By processing the tobacco prior to extracting nicotine and other organoleptic components, the tobacco-derived nicotine may include ingredients that provide a favorable experience. The tobacco-derived nicotine can be obtained by mixing cured tobacco or cured and fermented tobacco with water or another solvent (e.g., ethanol) followed by removing the insoluble tobacco material. The tobacco extract may be further concentrated or purified. In some cases, select tobacco constituents can be removed. Nicotine can also be extracted from tobacco in the methods described in the following patents: U.S. Pat. Nos. 2,162,738; 3,139,436; 3,396,735; 4,153,063; 4,448,208; and 5,487,792.

Liquid nicotine can be pure, substantially pure, or diluted prior to mixing it with soluble fiber. Soluble fiber dissolves in water at ambient temperature. Insoluble fiber does not dissolve in water at ambient temperature. Soluble fibers can attract water and form a gel. Not only are many soluble fibers safe for consumption, but some soluble fibers are used as a dietary supplement. As a dietary supplement, soluble fiber can slow down digestion and delay the emptying of a stomach. Instead of using soluble fiber as a mere additive, however, nicotine lozenges provided herein include a matrix of soluble fiber, which can dissolve to provide access to nicotine (and optionally other additives) included in the soluble-fiber matrix.

For liquid nicotine, a diluting step is optional. In some cases, liquid nicotine is diluted to a concentration of between 1 weight percent and 75 weight percent prior to mixing the liquid nicotine with soluble fiber. In some cases, liquid nicotine is diluted to a concentration of between 2 weight percent and 50 weight percent prior to mixing the liquid nicotine with soluble fiber. In some cases, liquid nicotine is diluted to a concentration of between 5 weight percent and 25 weight percent prior to mixing the liquid nicotine with soluble fiber. For example, liquid nicotine can be diluted to a concentration of about 10 weight percent prior to mixing the liquid nicotine with soluble fiber.

vi. Phosphodiesterase Type 5 Inhibitors

Phosphodiesterase type 5 inhibitors (PDE5 inhibitors) block the degradative action of cGMP-specific phosphodiesterase type 5 (PDE5) on cyclic GMP in the smooth muscle cells lining the blood vessels supplying the corpus cavernosum of the penis. These drugs, including vardenafil (Levitra®), sildenafil (Viagra®), and tadalafil (Cialis®), are administered orally for the treatment of erectile dysfunction and were the first effective oral treatment available for the condition.

PDE5 inhibitors have also been studied for other clinical use as well, including cardiovascular and heart diseases. For example, because PDE5 is also present in the arterial wall smooth muscle within the lungs, PDE5 inhibitors have also been explored for lung diseases such as pulmonary hypertension and cystic fibrosis. Pulmonary arterial hypertension, a disease characterized by sustained elevations of pulmonary artery pressure, which leads to an increased incidence of failure of the right ventricle of the heart, which in turn can result in the blood vessels in the lungs become overloaded with fluid. Two oral PDE5 inhibitors, sildenafil (Revatio®) and tadalafil (Adcirca®), are approved for the treatment of pulmonary arterial hypertension. PDE5 inhibitors have been found to have activity as both a corrector and potentiator of CFTR protein abnormalities in animal models of cystic fibrosis disease (Lubamba et al., Am. J. Respir. Crit. Care Med. (2008) 177:506-515, Lubamba et al., J. Cystic Fibrosis (2012) 11:266-273). Sildenafil has also been studied as a potential anti-inflammatory treatment for cystic fibrosis. Oral PDE5 inhibitors have also been reported to have anti-remodeling properties and to improve cardiac inotropism, independent of afterload changes, with a good safety profile (Giannetta et al., BMC Medicine (2014) 12:185). However, oral administration of PDE5 inhibitors results in poor and variable bioavailability and also extensive metabolism in the liver (Sandqvist et al., Eur. J. Cin. Pharmacol. (2013) 69:197-207; Mehrotra, Intl. J. Impotence Res. (2007) 19:253-264). If oral doses are increased beyond certain levels, the incidence of systemic side effects increase which prevents the acceptable use of these drugs. (Levitra EMEA Scientific Discussion Document, 2005).

Accordingly, in other aspects, within the compositions and methods of the present invention, the PDE5 inhibitor may include, but is not limited to, avanafil, lodenafil, mirodenafil, sildenafil (or analogs thereof, for example, actetildenafil, hydroxyacetildenafil, or dimethyl-sildenafil), tadalafil, vardenafil, udenafil, acetildenafil, or thiome-thisosildenafil. The structures of these compounds are respectively shown below:

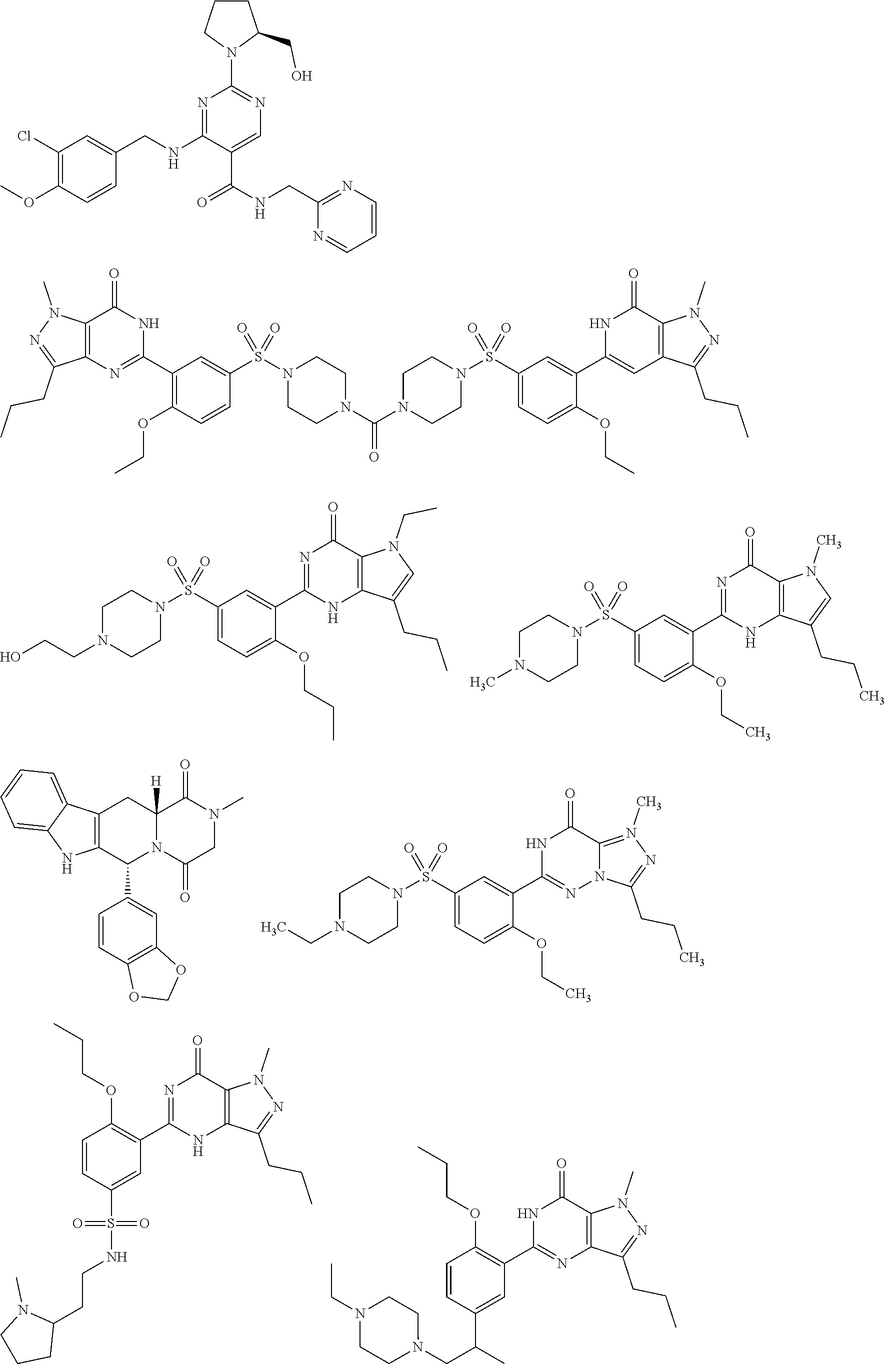

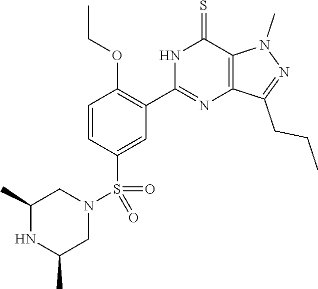 [SMILES: CCCc1nn(C)c2c(=S)[nH]c(-c3cc(S(=O)(=O)N4C[C@@H](C)N[C@@H](C)C4)ccc3OCC)nc12]

vii. Maca Extract

Lepidium meyenii (Maca, maca-maca, maino, ayak chichira, and ayak willku) is a Peruvian plant of the Brassicaceae family cultivated for more than 2000 years. Its main active principles are alkaloids (Macaridine, Lepidiline A and B); bencil-isotiocyanate and glucosinolates; macamides, beta-ecdysone and fitosterols. These substances activate ATP synthesis which confers energizing properties. They also diminish variations in homeostasis produced by stress because they reduce corticosterone's high levels; prevent glucose diminution and the increase of suprarenal glands' weight due to stress. They also restore homeostasis and improve energy (Lopez-Fando et al. (2004) Phytother Res. 18:471-4). A double blind placebo-controlled, randomized, parallel trial study in which active treatment with different doses of Lepidium meyenii was compared with placebo showed an improvement in sexual desire. (Gonzales et al. (2002) Andrologia 34:367-72). Lepidium meyenii also improves sperm production and sperm motility by mechanisms not related to LH, FSH, PRL, T and E2 (Gonzales et al. (2001) Asian J. Androl. 3:301-3).

viii. Steroid Hormones

In some embodiments, the active agent is a steroid, including hormones and sex hormones. The term “sex hormone” refers to natural or synthetic steroid hormones that interact with vertebrate androgen or estrogen receptors, such as estrogens, anti-oestrogens (or SERMs), androgens, anti-androgens, progestins, and mixtures thereof.

For example, steroid hormones suitable for use in the compositions described herein include the numerous natural and synthetic steroid hormones, including androgens, estrogens, and progestagens and derivatives thereof, such as dehydroepiandrosterone (DHEA), androstenedione, androstenediol, dihydrotestosterone, testosterone, progesterone, progestins, oestriol, oestradiol. Other suitable steroid hormones include glucocorticoids, thyroid hormone, calciferol, pregnenolone, aldosterone, cortisol, and derivatives thereof. Suitable steroid hormones especially include the sexual hormones having estrogenic, progestational, androgenic, or anabolic effects, such as estrogen, estradiol and their esters, e.g., the valerate, benzoate, or undecylate, ethinylestradiol, etc.; progestogens, such as norethisterone acetate, levonorgestrel, chlormadinone acetate, cyproterone acetate, desogestrel, or gestodene, etc.; androgens, such as testosterone and its esters (propionate, undecylate, etc.), etc.; anabolics, such as methandrostenolone, nandrolone and its esters.

a. Estrogens

Estrogens refer to a group of endogenous and synthetic hormones that are important for and used for tissue and bone maintenance. Estrogens are endocrine regulators in the cellular processes involved in the development and maintenance of the reproductive system. The role of estrogens in reproductive biology, the prevention of postmenopausal hot flashes, and the prevention of postmenopausal osteoporosis are well established. Estradiol is the principal endogenous human estrogen, and is found in both women and men.

The biological actions of estrogens and antiestrogens are manifest through two distinct intracellular receptors, estrogen receptor alpha (ERα) and estrogen receptor beta (ERβ). Endogenous estrogens are typically potent activators of both receptor subtypes. For example estradiol acts as an ERα agonist in many tissues, including breast, bone, cardiovascular and central nervous system tissues. Selective estrogen receptor modulators commonly act differently in different tissues. For example, a SERM may be an ERα antagonist in the breast, but may be a partial ERα agonist in the uterus, bone and cardiovascular systems. Compounds that act as estrogen receptor ligands are, therefore, useful in treating a variety of conditions and disorders.

As used herein, “estrogen” includes estrogenic steroids such as estradiol (17-β-estradiol), estradiol benzoate, estradiol 17 β-cypionate, estropipate, equilenin, equilin, estriol, estrone, ethinyl estradiol, conjugated estrogens, esterified estrogens, phytoestrogens, semi-natural estrogens such as estradiol valerate, synthetic estrogens such as ethinyl-estradiol, and mixtures thereof.

In some embodiments, a pharmaceutical composition is provided for topical administration to a skin surface comprising water, and at least one therapeutically active agent selected from the estrogens. In some embodiments the compositions and methods of the invention further comprise an alcohol and a fatty acid ester. In some embodiments, a pharmaceutical composition is provided for topical administration to a skin surface comprising water and at least one therapeutically active agent being estradiol. In some embodiments, the compositions and methods of the invention further comprise an alcohol and a fatty acid ester. In particular embodiments of such compositions when the active agent is estradiol, the compositions and methods do not further comprise the combination of progesterone, propylene glycol, oleic acid, ethyl oleate, ethanol, hydroxypropylcellulose and purified water.

b. Anti-Estrogens

Anti-estrogens are a class of pharmaceutically active agents now referred to as Selective Estrogen Receptors Modulators (SERMs), which were generally understood to be compounds capable of blocking the effect of estradiol without displaying any estrogenic activity of their own. Such a description is now known to be incomplete, however. The term SERM has been coined to describe compounds that, in contrast to pure estrogen agonists or antagonists, have a mixed and selective pattern of estrogen agonist-antagonist activity, which largely depends on the targeted tissue. The pharmacological goal of these drugs is to produce estrogenic actions in those tissues where these actions are beneficial (such as bone, brain, liver) and to have either no activity or antagonistic activity in tissues such as breast and endometrium, where estrogenic actions (cellular proliferation) might be deleterious.

In specific embodiments, the anti-estrogens (SERMs) are selected from the group consisting of endoxifen, droloxifene, clomifene, raloxifene, tamoxifen, 4-OH tamoxifen, toremifene, danazol, and pharmaceutically acceptable salts thereof. In a more particular embodiment, a pharmaceutical composition is provided for topical administration to a skin surface comprising water, at least one therapeutically active agent selected from the anti-oestrogens (SERMs) selected from the group consisting of clomifene, raloxifene, droloxifene, endoxifen or the pharmaceutically acceptable salts thereof, an alcohol, and a fatty acid ester.

In a particular embodiment, a pharmaceutical composition is provided for topical administration to a skin surface comprising water, at least one therapeutically active agent selected from the anti-estrogens (SERMs). In some aspects the composition further comprises an alcohol and a fatty acid ester.

c. Androgens

Testosterone is the main androgenic hormone formed in the testes. Testosterone therapy is currently indicated for the treatment of male hypogonadism. It is also under investigation for the treatment of wasting conditions associated with AIDS and cancer, testosterone replacement in men over the age of 60, osteoporosis, combination hormone replacement therapy for women and male fertility control.

Orally administered testosterone is largely degraded in the liver, and is therefore not a viable option for hormone replacement since it does not allow testosterone to reach systemic circulation. Further, analogues of testosterone modified to reduce degradation (e.g., methyltestosterone and methandrostenolone) have been associated with abnormalities in liver function, such as elevation of liver enzymes and conjugated bilirubin. Injected testosterone produces wide peak-to-trough variations in testosterone concentrations that do not mimic the normal fluctuations of testosterone, and makes maintenance of physiological levels in the plasma difficult. Testosterone injections are also associated with mood swings and increased serum lipid levels. Injections require large needles for intramuscular delivery, which leads to diminished patient compliance due to discomfort.

To overcome these problems, transdermal delivery approaches have been developed to achieve therapeutic effects in a more patient friendly manner. For example, U.S. Pat. No. 5,460,820 discloses a testosterone-delivering patch for delivering 50 to 500 μg/day of testosterone to a woman. In addition, U.S. Pat. No. 5,152,997 discloses a device comprising a reservoir of testosterone with a skin permeation enhancer and a means for maintaining the reservoir in diffusional communication with the skin, such as an adhesive carrier device or a basal adhesive layer.

In some embodiments, androgens may be selected from the group consisting of the natural androgen, testosterone, and its semi-natural or synthetic derivatives, for instance methyltestosterone; physiological precursors of testosterone such as dehydroepiandrosterone or DHEA, or alternatively prasterone and its derivatives, for instance DHEA sulphate, A-4-androstenedione and its derivatives; testosterone metabolites, for instance dihydrotestosterone (DHT) obtained after the enzymatic action of 5-α-reductases; or substances with an androgenic-type effect, such as tibolone. In some aspects the composition further comprises an alcohol and a fatty acid ester.

d. Anti-Androgens

In some embodiments, anti-androgens are selected from the group consisting of steroidal compounds such as cyproterone acetate and medroxyprogesterone, or non-steroidal compounds such as flutamide, nilutamide or bicalutamide. In some aspects the composition further comprises an alcohol and a fatty acid ester.

e. Progestins and Progesterone

The term “progesterone” as used herein refers to a member of the progestin family and comprises a 21 carbon steroid hormone. Progesterone is also known as D4-pregnene-3,20-dione; 4-pregnene-3,20-dione; or pregn-4-ene-3,20-dione. A progestin is a molecule whose structure is related to that of progesterone, is synthetically derived, and retains the biologically activity of progesterone. Representative synthetic progestin include, but are not limited to, modifications that produce 17a-OH esters (i.e., 17 a-hydroxyprogesterone caproate), as well as, modifications that introduce 6 a-methyl, 6-Me, 6-ene, and 6-chloro sustituents onto progesterone (i.e., medroxyprogesterone acetate, megestrol acetate, and chlomadinone acetate).

In some embodiments, progestin(s) used in the compositions and methods described herein may be selected from the group consisting of natural progestins, progesterone or its derivatives of ester type, and synthetic progestins of type 1, 2 or 3. The first group comprises molecules similar to progesterone or the synthetic progestins 1 (SP1) (pregnanes), for example the progesterone isomer (retroprogesterone), medrogesterone, and norprogesterone derivatives (demegestone or promegestone). The second group comprises 17α-hydroxy-progesterone derivatives or synthetic progestins 2 (SP2) (pregnanes), for example cyproterone acetate and medroxyprogesterone acetate. The third group comprises norsteroids or synthetic progestins 3 (SP3), (estranes or nor-androstanes). These are 19-nortestosterone derivatives, for example norethindrone. This group also comprises molecules of gonane type, which are derived from these nor-androstanes or estranes and have a methyl group at C18 and an ethyl group at C13. Examples that may be mentioned include norgestimate, desogestrel (3-ketodesogestrel) or gestodene. Tibolone, which has both progestin and androgenic activity, may also advantageously be selected in the compositions and methods described herein. In some aspects the composition further comprises an alcohol and a fatty acid ester. In some embodiments of such compositions, when the active agent is progesterone, the composition does not further comprise the combination of estradiol, propylene glycol, oleic acid, ethyl oleate, ethanol, hydroxypropylcellulose and purified water. In some embodiments, the therapeutically active agent in the compositions and methods is a progestin, an estrogen or a combination of the two.

ix. Fentanyl

Fentanyl (also known as fentanil) is a potent synthetic narcotic analgesic with a rapid onset and short duration of action. Fentanyl is a strong agonist at μ-opioid receptors. Fentanyl is manufactured under the trade names of SUBLIMAZE, ACTIQ, DUROGESIC, DURAGESIC, FENTORA, ONSOLIS INSTANYL, ABSTRAL, and others. Historically, fentanyl has been used to treat chronic breakthrough pain and is commonly used before procedures as an anesthetic in combination with a benzodiazepine. Fentanyl is approximately 100 times more potent than morphine with 100 micrograms of fentanyl approximately equivalent to 10 mg of morphine and 75 mg of pethidine (meperidine) in analgesic activity.

Suitable analogues of fentanyl include, without limitation, the following: alfentanil (trade name ALFENTA), an ultra-short-acting (five to ten minutes) analgesic; sufentanil (trade name SUFENTA), a potent analgesic for use in specific surgeries and surgery in heavily opioid-tolerant/opioid-dependent patients; remifentanil (trade name ULTIVA), currently the shortest-acting opioid, has the benefit of rapid offset, even after prolonged infusions; carfentanil (trade name WILDNIL) an analogue of fentanyl with an analgesic potency 10,000 times that of morphine and is used in veterinary practice to immobilize certain large animals such as elephants; and lofentanil an analogue of fentanyl with a potency slightly greater than carfentanil.

x. Bunrenornhine

Buprenorphine (17-(cyclopropyl-methyl)-α-(1,1-dimethylethyl)-4,5-epoxy-18,19-dihydro-3-hydroxy-6-methoxy-α-methyl-6,14-ethenomorphinan-7-methanol) is anendoethylene morphinan derivative and a partial agonist of μ-opioid receptors with a strong analgesic effect. Buprenorphine is a partially synthetic opiate whose advantage over other compounds from this class of substance lies in a higher activity. This means that freedom from pain can be achieved in cancer or tumour patients with very unfavourable diagnosis, in the final stage, with daily doses of around 1 mg. A feature of buprenorphine in this context over the synthetic opioid fentanyl and its analogues is that the addictive potential of buprenorphine is lower than that of these compounds. A disadvantage is that, owing to the high molecular weight of buprenorphine, namely 467.64 daltons, it has been traditionally been difficult to effect its transdermal absorption.

xi. Scopolamine

Scopolamine is a so-called antiemitic, it is preferably used to avoid nausea and vomiting, for example, arising from repeated passive changes in the balance occurring during traveling. Scopolamine is represented by the following chemical structure

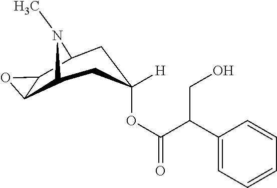

Scopolamine analogs are also encompassed by the compositions and methods of the present invention. It is understood that the phrase “scopolamine analogs” includes compounds that generally have the same backbone as scopolamine, but where various moieties have been substituted or replaced by other substituents or moieties. Some examples of scopolamine analogs that can be used in the compositions and methods disclosed herein include, but are not limited to, salts of scopolamine with various acids, such as hydrochloric acid, hydrobromic acid, hydroiodic acid, nitric acid, phosphoric acid, sulfuric acid, and the like. In one aspect, a suitable scopolamine analog can be scopolamine hydrobromide.

Additional examples of scopolamine analogs include, but are not limited to, N-alkylated analogs of scopolamine, that is, analogs containing an alkyl substituent attached to the nitrogen atom, forming a quaternary ammonium species. By “alkyl” is meant a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl, and the like. The alkyl group can also be substituted or unsubstituted.

Also included are other salts (e.g., pharmaceutically acceptable salts) of such N-alkylated scopolamine analogs.

Still further examples of scopolamine analogs include, but are not limited to, un-epoxylated analogs of scopolamine, that is, analogs where the epoxy group is removed. One example of such an analog is atropine. Like scopolamine, atropine has various salt and N-alkylated analogs. These atropine analogs are intended to be included by the phrase “scopolamine analogs.” As such, further examples of scopolamine analogs include, but are not limited to, analogs of atropine with various salts (e.g., atropine hydrobromide, atropine hydrochloride, and the like) and N-alkylated analogs of atropine (e.g., atropine methyl bromide). Also included are homatropine and its salts and N-alkylated analogs.

A list of suitable scopolamine analogs that can be used in the disclosed compositions and methods, including their commercial brand names, includes, but is not limited to, atropine, atropine hydrobromide, atropine oxide hydrochloride, atropine sulfate, belladonna, scopolamine, scopolamine hydrobromide, scopolamine methylbromide, scopolamine butylbromide, homatropine, ipratropium, tiotropium, hyoscyamine sulfate, methscopolamine, methscopolamine bromide, homatropine hydrobromide, homatropine methylbromide, hyoscyamine, hyoscyamine hydrobromide, hyoscyamine sulfate, propantheline bromide, anisotropine, anisotropine methylbromide, methantheline bromide, emepronium bromide, clindinium, clidinium bromide, hyoscine, hyoscine butylbromide, hyoscine hydrobromide, hyoscine methobromide, hyoscine methonitrite, hyoscyamine, hyoscyamine sulfate, buscapine, buscolysin, buscopan, butyiscopolamine, hyoscine N-butylbromide, N-butylscopolammonium bromide, scopolan bromide, butylscopolammonium bromide, N-butylscopolammonium chloride, hyoscine N-butylbromide, DD-234, hyoscine methiodide, hyoscine methobromide, methyiscopolamine nitrate, methylscopolammoium methylsulfate, N-methylscine methylsulfate, N-methylscopolamine bromide, N-methylscopolamine iodide, N-methylscopolamine methylchloride, N-methylscopolamine methylsulfate, N-methylscopolamine nitrate, skopyl, ulix bromide, N-methylscopolamine, N-methylscopolamine methobromide, scopolamine methylchloride, N-methylscine methylsulfate, tematropium methylsulfate, and N-isopropylatropine, including salts and derivatives thereof.

xii. Antioxidants

Antioxidants are chemicals that inhibit lipid oxidation. Some antioxidants (e.g., phenolic compounds) interrupt the free-radical chain of oxidative reactions by complexing with free radicals to form stable compounds that do not initiate or propagate further oxidation. Other antioxidants (e.g., acid compounds) slow the oxidative process by scavenging the reactive oxygen species. And still other antioxidants (e.g., chelators) slow oxidation by complexing with pro-oxidative metal ions.

Thousands of different types of antioxidants exist in nature. Some antioxidants of most importance to human health include without limitation astaxanthin, enzymes such as Superoxide Dismusase, vitamins A, C, and E, beta-carotene, selenium, lycopene, lutein, Coenzyme Q10, phytic acid, flavonoids, and polyphenols. Antioxidants are also separated into categories based upon whether they are water-soluble (hydrophilic) or fat-soluble (hydrophobic or lipophilic). Water-soluble antioxidants tend to predominantly react with oxidants in the cell cytosol and the blood plasma, while fat-soluble antioxidants tend to protect cell membranes from lipid peroxidation.

Various antioxidant compositions have been developed for the stabilization of oils and fats; most are mixtures of natural phenolic compounds (e.g., tocopherols) and acid compounds (e.g., ascorbic acid). While these antioxidant compositions inhibit lipid oxidation, they are not nearly as effective as synthetic phenolic antioxidants. One of the most effective antioxidants is ethoxyquin (6-ethoxy-1,2-dihydro-2,2,4-trimethylquinoline, sold under the trademark SANTOQUIN®), which is widely used as an antioxidant or preservative in feed supplements and a variety of other applications.

Several antioxidants are suitable for use in the compositions and methods of the present invention. The antioxidant may be a compound that interrupts the free-radical chain of oxidative reactions by protonating free radicals, thereby inactivating them. The antioxidant may be a compound that scavenges the reactive oxygen species. Alternatively, the antioxidant may be a compound that chelates the metal catalysts. The antioxidant may be a synthetic compound, a semi-synthetic compound, or a natural (or naturally-derived) compound.

In some aspects, the antioxidant is a substituted 1,2-dihydroquinoline. Substituted 1,2-dihydroquinoline compounds suitable for use in the invention generally correspond to Formula (I) as described in U.S. Patent App. Pub. No. US20080019860, particularly where the substituted 1,2-dihydroquinoline is 6-ethoxy-1,2-dihydro-2,2,4-trimethylquinoline (commonly known as ethoxyquin and sold under the trademark SANTOQUIN®) having the structure:

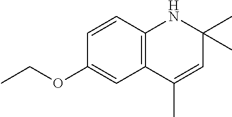 [SMILES: CCOc1ccc2c(c1)C(C)=CC(C)(C)N2]

In other aspects, the antioxidant includes, but is not limited to, ascorbic acid and its salts, ascorbyl palmitate, ascorbyl stearate, anoxomer, N-acetylcysteine, benzyl isothiocyanate, o-, m- or p-amino benzoic acid (o is anthranilic acid, p is PABA), butylated hydroxyanisole (BHA), butylated hydroxytoluene (BHT), caffeic acid, canthaxantin, alpha-carotene, beta-carotene, beta-caraotene, beta-apo-carotenoic acid, carnosol, carvacrol, catechins, cetyl gallate, chlorogenic acid, citric acid and its salts, clove extract, coffee bean extract, p-coumaric acid, 3,4-dihydroxybenzoic acid, N,N′-diphenyl-p-phenylenediamine (DPPD), dilauryl thiodipropionate, distearyl thiodipropionate, 2,6-di-tert-butylphenol, dodecyl gallate, edetic acid, ellagic acid, erythorbic acid, sodium erythorbate, esculetin, esculin, 6-ethoxy-1,2-dihydro-2,2,4-trimethylquinoline, ethyl gallate, ethyl maltol, ethylenediaminetetraacetic acid (EDTA), eucalyptus extract, eugenol, ferulic acid, flavonoids, flavones (e.g., apigenin, chrysin, luteolin), flavonols (e.g., datiscetin, myricetin, daemfero), flavanones, fraxetin, fumaric acid, gallic acid, gentian extract, gluconic acid, glycine, gum guaiacum, hesperetin, alpha-hydroxybenzyl phosphinic acid, hydroxycinammic acid, hydroxyglutaric acid, hydroquinone, N-hydroxysuccinic acid, hydroxytryrosol, hydroxyurea, ice bran extract, lactic acid and its salts, lecithin, lecithin citrate; R-alpha-lipoic acid, lutein, lycopene, malic acid, maltol, 5-methoxy tryptamine, methyl gallate, monoglyceride citrate; monoisopropyl citrate; morin, beta-naphthoflavone, nordihydroguaiaretic acid (NDGA), octyl gallate, oxalic acid, palmityl citrate, phenthiazine, phosphatidylcholine, phosphoric acid, phosphates, phytic acid, phy tylubichromel, pimento extract, propyl gallate, polyphosphates, quercetin, trans-resveratrol, rosemary extract, rosmarinic acid, sage extract, sesarnol, siyarin, sinapic acid, succinic acid, stearyl citrate, syringic acid, tartaric acid, thymol, tocopherols (i.e., alpha-, beta-, gamma- and delta-tocopherol), tocotrienols (i.e., alpha-, beta, gamma- and delta-tocotrienols), tyrosol, vanilic acid, 2,6-di-tert-butyl-4-hydroxymethylphenol (i.e., lonox 100) 2,4-(tris-3′,5′ bi-tert-butyl-4′-hydroxybenzyl)-mesitylene (i.e., lonox 330), 2,4,5-trihydroxybutyrophenone, ubiquinone, tertiary butyl hydroquinone (TBHQ), thiodipropionic acid, trihydroxy butyrophenone, tryptamine, tyramine, uric acid, vitamin K and derivates, vitamin Q10, wheat germ oil, zeaxanthin, or combinations thereof.

Further exemplary antioxidants include synthetic phenolic compounds, such as tertiary butyl hydroquinone (TBHQ); gallic acid derivatives, such as n-propyl gallate; vitamin C derivatives, such as ascorbyl palmitate; lecithin; and vitamin E compounds, such as, alpha-tocopherol.

xii. Imaging Agents

The described methods may also be used to deliver imaging agents to the brain of a subject. Imaging agents that might not readily access the brain on their own may also be delivered using the described methods. For example, in some embodiments the described methods may be used to deliver the imaging agent Cu64 to the brain of a subject to allow for imaging. Further, the described methods may be used to deliver a combination of one or more therapeutic agents, imaging agents, or both therapeutic agents and imaging agents to the brain of a subject.

G. Lyophylization

In a further aspect, the food product infused with a lipophilic active agent of the present invention is lyophilized. Lyophilization, also known as freeze-drying, is a process whereby water is sublimed from a composition after it is frozen. The frozen solution is then typically subjected to a primary drying step in which the temperature is gradually raised under vacuum in a drying chamber to remove most of the water, and then to a secondary drying step typically at a higher temperature than employed in the primary drying step to remove the residual moisture in the lyophilized composition. The lyophilized composition is then appropriately sealed and stored for later use. Tang et al. (2004) Pharmaceutical Research 21:191-200 describes the scientific principles pertaining to freeze drying and guidelines for designing suitable freeze drying processes. Further description of freeze drying is found in Remington (2006) The Science and Practice of Pharmacy, 21^{st} edition, Lippincott Williams & Wilkins, pp. 828-831.

H. Methods of Treating Central Nervous System Disorders

A method is also provided for treating a central nervous system disease, disorder, or condition comprising administering the edible product infused with a lipophilic active agent or the beverage product infused with a lipophilic active agent to a subject in need thereof, and wherein the central nervous system disease, disorder, or condition is selected from the group consisting of a metabolic disease, a behavioral disorder, a personality disorder, dementia, a cancer, a neurodegenerative disorder, pain, a viral infection, a sleep disorder, and an arteriovenous malformation, a brain aneurysm, a brain tumor, a spinal cord tumor, facial paralysis, a pituitary disorder, a stroke, and a seizure disorder.

In some embodiments, the central nervous system disease, disorder, or condition is selected from the group consisting of Amyotrophic Lateral Sclerosis (ALS), Multiple Sclerosis, Parkinson's Disease, Fabry disease, Wernicke-Korsakoff syndrome, Alzheimer's disease, Huntington's disease, Lewy Body disease, Canavan disease, Hallervorden-Spatz disease, and Machado-Joseph disease.

In some embodiments, the central nervous system disease, disorder, or condition is selected from the group consisting of acid lipase disease, attention deficit hyperactivity disorder (ADHD), an anxiety disorder, borderline personality disorder, bipolar disorder, depression, an eating disorder, obsessive-compulsive disorder, schizophrenia, Barth syndrome, Tourette's syndrome, and Restless Leg syndrome.

In some embodiments, the pain is selected from the group consisting of neuropathic pain, central pain syndrome, somatic pain, visceral pain, and headache.

A method is also provided for enhancing the delivery of a lipophilic agent across the blood brain barrier of a subject, comprising administering the edible product infused with a lipophilic active agent or the beverage product infused with a lipophilic active agent to a subject in need thereof. In some embodiments, the edible product or the beverage product is heated to a temperature that is greater than or equal to human body temperature.

In a further aspect, a method of enhancing the bioavailability of a lipophilic active agent is provided, comprising heating any of the compositions disclosed herein to a temperature that is greater than or equal to human body temperature. In some aspects, oral administration of any of the compositions disclosed herein to a subject in need thereof results in a heating of the compositions to a temperature that is equal to human body temperature.

In another aspect, a method of administering any of the lipophilic active agents described herein to a subject is provided, comprising oral administration of any of the compositions of the present invention. Such administration may be for any purpose, including overall health and wellness, mental acuity, alertness, recreation, and the like.

In another aspect, a method is provided of treating a central nervous system disease, disorder, or condition in a subject in need thereof, comprising administering a lipophilic active agent infused food product with enhanced delivery across the blood-brain barrier to the subject, wherein the lipophilic active agent infused food product with enhanced delivery across the blood-brain barrier is produced by the steps of:
(a) contacting a food product with an oil comprising a lipophilic active agent and a bioavailability enhancing agent, wherein the bioavailability enhancing agent comprises an edible oil comprising long chain fatty acids; and
(b) dehydrating the food product;
   thereby producing a lipophilic active agent infused food product with enhanced delivery across the blood-brain barrier; wherein the lipophilic active agent infused food product with enhanced delivery across the blood-brain barrier produces a concentration of lipophilic active agent in central nervous system tissue in a subject that is greater than the concentration of lipophilic active agent in central nervous system tissue in the subject in the absence of the edible oil comprising long chain fatty acids; and further wherein:
(i) the lipophilic active agent is selected from the group consisting of cannabinoids, terpenes and terpenoids, NSAIDs, vitamins, nicotine, phosphodiesterase type 5 (PDE5) inhibitors, Maca extract, estrogen, progestin, testosterone, buprenorphine, and scopolamine; and
(ii) the food product is selected from the group consisting of tea leaves, coffee beans, cocoa powder, meats, fish, fruits, vegetables, dairy products, legumes, pastas, breads, grains, seeds, nuts, spices, and herbs.

In another aspect, a method is provided of treating a central nervous system disease, disorder, or condition in a subject in need thereof, comprising administering a ready-to-drink beverage composition comprising a lipophilic active agent with enhanced delivery across the blood-brain barrier to the subject, wherein the ready-to-drink beverage composition comprising a lipophilic active agent with enhanced delivery across the blood-brain barrier is produced by the steps of:
(a) contacting an emulsifier with an oil comprising a lipophilic active agent and a bioavailability enhancing agent, thereby producing a mixture comprising the emulsifier, the oil comprising the lipophilic active agent, and the bioavailability enhancing agent;
(b) dehydrating the mixture, thereby producing a dehydrated mixture comprising the emulsifier, the oil comprising the lipophilic active agent, and the bioavailability enhancing agent; and
(c) combining the dehydrated mixture with a ready-to-drink beverage composition, thereby producing the ready-to-drink beverage composition comprising the lipophilic active agent with enhanced delivery across the blood-brain barrier;
   wherein:
(i) the bioavailability enhancing agent comprises an edible oil comprising long chain fatty acids;
(ii) the ready-to-drink beverage composition comprising a lipophilic active agent with enhanced delivery across the blood-brain barrier produces a concentration of lipophilic active agent in central nervous system tissue in a subject that is greater than the concentration of lipophilic active agent in central nervous system tissue in the subject in the absence of the edible oil comprising long chain fatty acids; and
(iii) the lipophilic active agent is selected from the group consisting of cannabinoids, terpenes and terpenoids, NSAIDs, vitamins, nicotine, phosphodiesterase type 5 (PDE5) inhibitors, Maca extract, estrogen, progestin, testosterone, buprenorphine, and scopolamine.

In other aspects within the methods of treating a central nervous system disease, disorder, or condition in a subject in need thereof, the central nervous system disease, disorder, or condition (which encompasses psychiatric/behavioral diseases or disorders) may include, but is not limited to, acquired epileptiform aphasia, acute disseminated encephalomyelitis, adrenoleukodystrophy, agenesis of the corpus callosum, agnosia, aicardi syndrome, Alexander disease, Alpers' disease, alternating hemiplegia, Alzheimer's disease, amyotrophic lateral sclerosis, anencephaly, Angelman syndrome, angiomatosis, anoxia, aphasia, apraxia, arachnoid cysts, arachnoiditis, Arnold-chiari malformation, arteriovenous malformation, Asperger's syndrome, ataxia telangiectasia, attention deficit hyperactivity disorder, autism, auditory processing disorder, autonomic dysfunction, back pain, Batten disease, Behcet's disease, Bell's palsy, benign essential blepharospasm, benign focal amyotrophy, benign intracranial hypertension, bilateral frontoparietal polymicrogyria, binswanger's disease, blepharospasm, Bloch-sulzberger syndrome, brachial plexus injury, brain abscess, brain damage, brain injury, brain tumor, spinal tumor, Brown-sequard syndrome, canavan disease, carpal tunnel syndrome (cts), causalgia, central pain syndrome, central pontine myelinolysis, centronuclear myopathy, cephalic disorder, cerebral aneurysm, cerebral arteriosclerosis, cerebral atrophy, cerebral gigantism, cerebral palsy, charcot-marie-tooth disease, chiari malformation, chorea, chronic inflammatory demyelinating polyneuropathy (“CIDP”), chronic pain, chronic regional pain syndrome, Coffin lowry syndrome, coma (including persistent vegetative state), congenital facial diplegia, corticobasal degeneration, cranial arteritis, craniosynostosis, Creutzfeldt-jakob disease, cumulative trauma disorders, Cushing's syndrome, cytomegalic inclusion body disease (“CIBD”), cytomegalovirus infection, dandy-walker syndrome, Dawson disease, de morsier's syndrome, Dejerine-klumpke palsy, Dejerine-sottas disease, delayed sleep phase syndrome, dementia, dermatomyositis, developmental dyspraxia, diabetic neuropathy, diffuse sclerosis, dysautonomia, dyscalculia, dysgraphia, dyslexia, dystonia, early infantile epileptic encephalopathy, empty sella syndrome, encephalitis, encephalocele, encephalotrigeminal angiomatosis, encopresis, epilepsy, Erb's palsy, erythromelalgia, essential tremor, Fabry's disease, Fahr's syndrome, fainting, familial spastic paralysis, febrile seizures, fisher syndrome, Friedreich's ataxia, Gaucher's disease, Gerstmann's syndrome, giant cell arteritis, giant cell inclusion disease, globoid cell leukodystrophy, gray matter heterotopia, Guillain-barre syndrome, htlv-1 associated myelopathy, Hallervorden-spatz disease, head injury, headache, hemifacial spasm, hereditary spastic paraplegia, heredopathia atactica polyneuritiformis, herpes zoster oticus, herpes zoster, hirayama syndrome, holoprosencephaly, Huntington's disease, hydranencephaly, hydrocephalus, hypercortisolism, hypoxia, immune-mediated encephalomyelitis, inclusion body myositis, incontinentia pigmenti, infantile phytanic acid storage disease, infantile refsum disease, infantile spasms, inflammatory myopathy, intracranial cyst, intracranial hypertension, Joubert syndrome, Kearns-sayre syndrome, Kennedy disease, kinsbourne syndrome, Klippel feil syndrome, Krabbe disease, Kugelberg-welander disease, kuru, lafora disease, Lambert-eaton myasthenic syndrome, Landau-kleffner syndrome, lateral medullary (Wallenberg) syndrome, learning disabilities, leigh's disease, Lennox-gastaut syndrome, Lesch-nyhan syndrome, leukodystrophy, lewy body dementia, lissencephaly, locked-in syndrome, Lou Gehrig's disease, lumbar disc disease, lyme disease--neurological sequelae, machado-joseph disease (spinocerebellar ataxia type 3), macrencephaly, megalencephaly, Melkersson-rosenthal syndrome, Meniere's disease, meningitis, Menkes disease, metachromatic leukodystrophy, microcephaly, migraine, Miller Fisher syndrome, mini-strokes, mitochondrial myopathies, mobius syndrome, monomelic amyotrophy, motor neurone disease, motor skills disorder, moyamoya disease, mucopolysaccharidoses, multi-infarct dementia, multifocal motor neuropathy, multiple sclerosis, multiple system atrophy with postural hypotension, muscular dystrophy, myalgic encephalomyelitis, myasthenia gravis, myelinoclastic diffuse sclerosis, myoclonic encephalopathy of infants, myoclonus, myopathy, myotubular myopathy, myotonia congenita, narcolepsy, neurofibromatosis, neuroleptic malignant syndrome, neurological manifestations of aids, neurological sequelae of lupus, neuromyotonia, neuronal ceroid lipofuscinosis, neuronal migration disorders, niemann-pick disease, non 24-hour sleep-wake syndrome, nonverbal learning disorder, O'sullivan-mcleod syndrome, occipital neuralgia, occult spinal dysraphism sequence, ohtahara syndrome, olivopontocerebellar atrophy, opsoclonus myoclonus syndrome, optic neuritis, orthostatic hypotension, overuse syndrome, palinopsia, paresthesia, Parkinson's disease, paramyotonia congenita, paraneoplastic diseases, paroxysmal attacks, parry-romberg syndrome (also known as rombergs syndrome), pelizaeus-merzbacher disease, periodic paralyses, peripheral neuropathy, persistent vegetative state, pervasive developmental disorders, photic sneeze reflex, phytanic acid storage disease, pick's disease, pinched nerve, pituitary tumors, pmg, polio, polymicrogyria, polymyositis, porencephaly, post-polio syndrome, postherpetic neuralgia (“PHN”), postinfectious encephalomyelitis, postural hypotension, Prader-willi syndrome, primary lateral sclerosis, prion diseases, progressive hemifacial atrophy (also known as Romberg's syndrome), progressive multifocal leukoencephalopathy, progressive sclerosing poliodystrophy, progressive supranuclear palsy, pseudotumor cerebri, ramsay-hunt syndrome (type I and type II), Rasmussen's encephalitis, reflex sympathetic dystrophy syndrome, refsum disease, repetitive motion disorders, repetitive stress injury, restless legs syndrome, retrovirus-associated myelopathy, rett syndrome, Reye's syndrome, Romberg's syndrome, rabies, Saint Vitus' dance, Sandhoff disease, schizophrenia, Schilder's disease, schizencephaly, sensory integration dysfunction, septo-optic dysplasia, shaken baby syndrome, shingles, Shy-drager syndrome, Sjogren's syndrome, sleep apnea, sleeping sickness, snatiation, Sotos syndrome, spasticity, spina bifida, spinal cord injury, spinal cord tumors, spinal muscular atrophy, spinal stenosis, Steele-richardson-olszewski syndrome, see progressive supranuclear palsy, spinocerebellar ataxia, stiff-person syndrome, stroke, Sturge-weber syndrome, subacute sclerosing panencephalitis, subcortical arteriosclerotic encephalopathy, superficial siderosis, sydenham's chorea, syncope, synesthesia, syringomyelia, tardive dyskinesia, Tay-sachs disease, temporal arteritis, tetanus, tethered spinal cord syndrome, Thomsen disease, thoracic outlet syndrome, tic douloureux, Todd's paralysis, Tourette syndrome, transient ischemic attack, transmissible spongiform encephalopathies, transverse myelitis, traumatic brain injury, tremor, trigeminal neuralgia, tropical spastic paraparesis, trypanosomiasis, tuberous sclerosis, vasculitis including temporal arteritis, Von Hippel-lindau disease (“VHL”), Viliuisk encephalomyelitis (“VE”), Wallenberg's syndrome, Werdnig-hoffman disease, west syndrome, whiplash, Williams syndrome, Wilson's disease, and Zellweger syndrome. It is thus appreciated that all CNS-related states and disorders could be treated through the BBB route of drug delivery.

In some embodiments, a CNS disease, disorder, or condition according to embodiments of the present invention may be selected from a metabolic disease, a behavioral disorder, a personality disorder, dementia, a cancer, a neurodegenerative disorder, pain, a viral infection, a sleep disorder, a seizure disorder, acid lipase disease, Fabry disease, Wernicke-Korsakoff syndrome, ADHD, anxiety disorder, borderline personality disorder, bipolar disorder, depression, eating disorder, obsessive-compulsive disorder, schizophrenia, Alzheimer's disease, Barth syndrome and Tourette's syndrome, Canavan disease, Hallervorden-Spatz disease, Huntington's disease, Lewy Body disease, Lou Gehrig's disease, Machado-Joseph disease, Parkinson's disease, or Restless Leg syndrome. In some embodiments, the CNS disease, disorder, or condition is pain and is selected from neuropathic pain, central pain syndrome, somatic pain, visceral pain, and/or headache.

As used herein, the term “subject” treated by the presently disclosed methods in their many aspects is desirably a human subject, although it is to be understood that the methods described herein are effective with respect to all vertebrate species, which are intended to be included in the term “subject.” Accordingly, a “subject” can include a human subject for medical purposes, such as for the diagnosis or treatment of an existing disease, disorder, condition or the prophylactic diagnosis or treatment for preventing the onset of a disease, disorder, or condition or an animal subject for medical, veterinary purposes, or developmental purposes. Suitable animal subjects include mammals including, but not limited to, primates, e.g., humans, monkeys, apes, gibbons, chimpanzees, orangutans, macaques and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, guinea pigs, and the like. An animal may be a transgenic animal. In some aspects, the subject is a human including, but not limited to, fetal, neonatal, infant, juvenile, and adult subjects. Further, a “subject” can include a patient afflicted with or suspected of being afflicted with a disease, disorder, or condition. Thus, the terms “subject” and “patient” are used interchangeably herein. Subjects also include animal disease models (e.g., rats or mice used in experiments, and the like).

The term “effective amount,” as in “a therapeutically effective amount,” of a therapeutic agent refers to the amount of the agent necessary to elicit the desired biological response. As will be appreciated by those of ordinary skill in this art, the effective amount of an agent may vary depending on such factors as the desired biological endpoint, the agent to be delivered, the composition of the pharmaceutical composition, the target tissue or cell, and the like. More particularly, the term “effective amount” refers to an amount sufficient to produce the desired effect, e.g., to reduce or ameliorate the severity, duration, progression, or onset of a disease, disorder, or condition, or one or more symptoms thereof; prevent the advancement of a disease, disorder, or condition, cause the regression of a disease, disorder, or condition; prevent the recurrence, development, onset or progression of a symptom associated with a disease, disorder, or condition, or enhance or improve the prophylactic or therapeutic effect(s) of another therapy.

Actual dosage levels of the active ingredients in the presently disclosed compositions can be varied so as to obtain an amount of the active ingredient that is effective to achieve the desired therapeutic response for a particular subject, composition, route of administration, and disease, disorder, or condition without being toxic to the subject. The selected dosage level will depend on a variety of factors including the activity of the particular composition employed, the route of administration, the time of administration, the rate of excretion of the particular composition being employed, the duration of the treatment, other drugs, and/or materials used in combination with the particular composition employed, the age, sex, weight, condition, general health and prior medical history of the patient being treated, and like factors well known in the medical arts.

A physician having ordinary skill in the art can readily determine and prescribe the effective amount of the presently disclosed composition required. Accordingly, the dosage range for administration may be adjusted by the physician as necessary, as described more fully elsewhere herein.

### II. Kits and Containers

Also contemplated are kits that include any one of the compositions disclosed throughout the specification and claims. In certain embodiments, the composition is comprised in a container. The container can be a bottle, dispenser, or package. The container can dispense a pre-determined amount of the composition. The container can include indicia on its surface. The indicia can be a word, an abbreviation, a picture, or a symbol.

### EXAMPLES

### Example 1

A line of CBD and/or THC infused tea bags coming in a variety of flavors was developed.

I. Ingredients

Tea in leaf form, oil form, brewed form, organic and inorganic

Evaporated dry non-fat milk

CBD oil

Hemp oil or compatible oil for ingestion

Cannabis leaves, buds, oils; all strains with THC and/or CBD

II. ViPova® Formulas

II A. CBD Tea

Combine evaporated nonfat, dry milk with any and all teas, organic and inorganic

Blend CBD oil with the tea leaves

Dehydrate mixture of tea, CBD oil, and evaporated nonfat dry milk in a food dehydrator

End-product is ViPova® Tea with CBD enhancement only

II B. THC/CBD Tea

Combine evaporated nonfat, dry milk with any and all teas, organic and inorganic

Blend hemp or other ingestible oil with the tea leaves

Add Cannabis leaves to above mixture

Dehydrate mixture of tea, hemp or other ingestible oil, Cannabis leaves, and evaporated nonfat dry milk

End-product is ViPova® Tea with THC and CBD

III. ViPova® Formulas: Specifications

III A. CBD Tea

Tea: one tea bag contains 1 gram to 3 grams of tea leaves (dry weight)

Evaporated dry non-fat milk: 0.10-1.00 grams

CBD oil: 10 mgs.-25 mgs. per tea bag

III B. THC/CBD Tea

Tea: one tea bag contains 1.5-12 grams tea leaves (dry weight) per tea bag

Evaporated dry milk: 0.10-6.00 grams per tea bag

Hemp oil or other ingestible oil: 10 mgs.-25 mgs. per tea bag

Cannabis leaves: 1.00-12.00 grams per tea bag

III C. Production Equipment:

Commercial grinder for tea and/or Cannabis leaves

Commercial mixer

Commercial dehydrator

Commercial tea bag filling machine

IV. Flavorings

ViPova® Teas will provide a menu of flavorings for addition to tea bags or loose tea selections including, but not limited to mint, citrus, and vanilla.

### Example 2

A process for adhering CBD and/or THC to food products was developed. The food products may be selected from the group consisting of meats, fish, fruits, vegetables, dairy products, legumes, pastas, breads, grains, seeds, nuts, spices, and herbs. The process may or may not involve contacting the food product with sunflower and/or dry evaporated milk. The process involved the steps of:

1. A food product was saturated with 0-60 grams of CBD and/or THC oil or extract.

2. The food product was placed on dehydrator paper and placed in a food dehydrator for 0-24 hours.

3. The food product was removed from the dehydrator and stored in air-tight containers.

### Example 3

Black tea was formulated with various lipophilic active agents. Active agents were dosed into the tea at a concentration of approximately 4.5 mg of active ingredient per gram of finished product, using non-fat dry milk and sunflower seed oil as excipients. The following ingredients were used for the formulation:

453 g of loose leaf black tea

2265 mg active agent

45 g of instant non-fat dry evaporated milk

1132.5 mg of sunflower seed oil

Ingredients were combined in a stainless steel bowl and mixed with gloved hands.

A homogenous mixture was spread evenly on a dehydrator tray and dehydrated for 30 minutes.
After cooling, the formulated tea was placed into a sterile zip-lock bag.

The active ingredients that were formulated were: ASA (aspirin), ibuprofen, acetaminophen, diclofenac, indomethacin, piroxicam, nicotine, and vitamin E (α-tocopherol). The specific supplier information and lot numbers for each active agent are shown below in Table 1.

TABLE 1
Active Agents Used for Formulations

| | | | Catalogue | |
|---|---|---|---|---|
| Compound | CAS Number | Supplier | Number | Lot Number |
| ASA (aspirin) | 50-78-2 | Sigma-Aldrich | A2093 | #MKBQ8444V |
| Ibuprofen | 15687-27-1 | Sigma-Aldrich | I4883 | #MKBQ4505V |
| Acetaminophen | 103-90-2 | Sigma-Aldrich | A5000 | #MKBS7142V |
| Diclofenac | 15307-79-6 | Sigma-Aldrich | D6899 | #BCBN3367V |
| Indomethacin | 53-86-1 | Sigma-Aldrich | I8280 | #MKBR4530V |
| Piroxicam | 36322-90-4 | Sigma-Aldrich | P0847 | #SLBF3478V |
| Nicotine | 54-11-5 | Sigma-Aldrich | N3876 | #1449194V |
| Vitamin E | 10191-41-0 | Sigma-Aldrich | 258024 | #MKBT5983V |
| (α-tocopherol) | | | | |

The Tea used was loose leaf English Breakfast Tea from Upton Tea Imports (Holliston, Mass.).

The Sunflower Oil was Whole Foods brand organic sunflower oil.

The non-fat dry milk power was NowFoods brand organic non-fat dry milk.

The dehydrator used was a Presto Dehydrator, model #06300.

Each component of the formulation was weighed out and combined as described in the above procedure. The weights of the individual active agents for each formulation are summarized below in Table 2.

TABLE 2
Formulation of Active Agents

| | Compound | Non-Fat | Sunflower | | | Compound |
|---|---|---|---|---|---|---|
| Compound | Weight | Dry Milk | Seed Oil | Black Tea | Yield | Concentration |
| ASA (aspirin) | 2267.1 mg | 45.09 g | 1135 mg | 453.2 g | 479.3 g | 4.52 mg/g |
| Ibuprofen | 2265.5 mg | 45.05 g | 1138 mg | 453.8 g | 488.1 g | 4.51 mg/g |
| Acetaminophen | 2264.7 mg | 45.01 g | 1136 mg | 453.2 g | 477.9 g | 4.51 mg/g |
| Diclofenac | 2265.3 mg | 45.06 g | 1133 mg | 453.1 g | 441.3 g | 4.52 mg/g |
| Indomethacin | 2266.3 mg | 44.99 g | 1138 mg | 453.1 g | 491.5 g | 4.52 mg/g |
| Piroxicam | 2265.9 mg | 45.25 g | 1134 mg | 453.6 g | 488.3 g | 4.51 mg/g |
| Nicotine | 2264.9 mg | 45.02 g | 1133 mg | 453.1 g | 488.1 g | 4.52 mg/g |
| Vitamin E | 2271.1 mg | 45.05 g | 1135 mg | 453.2 g | 480.2 g | 4.53 mg/g |
| (α-tocopherol) | | | | | | |

For each formulation, the constituents were mixed by hand until a homogeneous mixture was achieved, then spread evenly on dehydrator trays for drying. Each formulation was dried for 30 minutes in dehydrator. After cooling, mixture was placed into Zip-Lock bag. After taring the analytical balance for the Zip-Lock bag, the weight of the final formulation was recorded and the concentration of active ingredient in the formulation calculated (Table 2).

### Example 4

As used herein, compositions incorporating DEHYDRATECH™ are compositions that incorporate a dehydrated mixture comprising a therapeutically effective amount of a lipophilic active agent and an edible oil comprising long chain fatty acids, particularly wherein dehydrated mixture is obtainable by the steps of:
i) combining a therapeutically effective amount of the lipophilic active agent with the edible oil comprising long chain fatty acids; and
ii) dehydrating the product of step (i), thereby producing the dehydrated mixture.

This study was designed to principally assess the relative ingestible nicotine absorption performance of DEHYDRATECH™-powered formulations compared to concentration-matched control formulations that lacked any form of delivery enabling technology in rats. Nicotine was administered in a nicotine polacrilex derivative format as is widely commercialized today in nicotine replacement therapy products such as chewing gums. Twelve male rats were divided into four groups of three, such that DEHYDRATECH™ and control formulations were each tested at a 1 mg/Kg and 10 mg/Kg dosage level. Formulations were administered orally and all rats were cannulated for blood collection at multiple intervals over an 8 hour duration post-dosing with the first data collection at the 15-minute mark. Urine and feces were also collected for up to a 24-hour duration post-dosing, and essential organ tissue samples were also collected for examination after the study. All samples were subjected to analytical testing in order to quantify the levels of nicotine therein, as well as the levels of three major liver metabolites thereof, hydroxycotinine, nicotine N′-oxide and cotinine, in order to assess the relative metabolite levels absorbed by the different formulations.

### Results & Observations

The DEHYDRATECH™ formulations generally achieved faster absorption, higher peak absorption and higher overall quantities of nicotine, on average, in the blood than the concentration-matched control formulations at both the 1 mg and 10 mg/Kg doses tested. Furthermore, as previously reported, there were no obvious signs of gastrointestinal distress such as vomiting or diarrhea indicating that the animals appeared to tolerate the treatment well.

Nicotine blood levels were evaluated multiple times over a period of 8 hours after dosing. In the 10 mg/Kg dosing arm, the control formulation required nearly 3 hours to reach similar levels of blood absorption that the DEHYDRATECH™ formulation reached in only 15 minutes. Furthermore, the DEHYDRATECH™ formulation went on thereafter to demonstrate peak plasma levels that were 148% of those achieved by the control formulation. If replicated in human studies, these findings are suggestive that DEHYDRATECH™'s technology could prove more effective in elevating blood nicotine levels through edible formats much more quickly and substantially than previously theorized, potentially making ingestible nicotine preparations a viable alternative to today's available product formats while also leading to a more rapid nicotine craving satiation.

Analysis of the liver metabolites revealed, as expected, that overall levels in the blood of two of the three metabolites studied were higher in the control group than in the DEHYDRATECH™ formulation group at the 10 mg/Kg dose. This result was especially pronounced in the 45-minute to 2-hour time interval post-dosing which is consistent with the expected timing of release of metabolites in higher quantity into the bloodstream by the liver following normal physiological processing of ingested nicotine with the control preparation, compared to the DehydraTECH™ technology that is believed to elude first pass liver metabolism. The DEHYDRATECH™ formulation also demonstrated lower quantities of nicotine in the rat urine at both doses, which is consistent with the fact that the levels of nicotine in the rat blood remained higher over the duration of the study with the DEHYDRATECH™ formulation than with the control. The study also revealed that the DEHYDRATECH™ formulation at the 10 mg/Kg level achieved up to 5.6-times as much nicotine upon analysis of the rat brain tissue than was recovered with the matching control formulation. These findings together perhaps suggest prolongation of nicotine effectiveness with the DEHYDRATECH™ formulation which may also be beneficial in humans to control cravings over an extended time-period from a single edible nicotine dose.

### Example 5

In this study, the exposure and distribution of nicotine and its major metabolites were evaluated following oral administration of two separate formulations (Reference and Test Nicotine Polacrilex) in male Sprague-Dawley rats.

Formulations were administered orally (PO) at 10 mg/kg. Following dosing, blood samples were collected up to 1 hour post dose; and urine and fecal samples were collected up to 24 hours post dose. Brain, liver, and kidney tissue were collected at 1 hour (Groups 1 & 5), 4 hours (Groups 2 & 6), following the 8 hour urine and feces sample collection (Groups 3 & 7), or following the 24 hour urine and feces sample collection (Groups 4 & 8). Blood, urine, feces, and tissue concentrations of each analyte were determined by LC-MS/MS. Plasma pharmacokinetic parameters were determined using WinNonlin (v8.0). Brain, liver, and kidney pharmacokinetic parameters were determined using WinNonlin (v8.0) software with sparse sampling.

Following PO dosing of Reference Nicotine Polacrilex at 10 mg/kg (Group 1), maximum plasma concentrations (average of 144±68.2 ng/mL) of nicotine were observed between 30 minutes and 1 hour post dosing. The average half-life after oral dosing could not be determined. The average exposure for nicotine (Group 1) based on the dose normalized AUClast was 8.71±2.76 hr*kg*ng/mL/mg.

Following PO dosing of Reference Nicotine Polacrilex at 10 mg/kg (Group 1), maximum plasma concentrations (average of 9.79±3.56 ng/mL) of hydroxycotinine metabolite were observed between 45 minutes and 1 hour post dosing. The average half-life after oral dosing could not be determined. The average exposure for hydroxycotinine (Group 1) based on the dose normalized AUClast was 0.420±0.146 hr*kg*ng/mL/mg.

Following PO dosing of Reference Nicotine Polacrilex at 10 mg/kg (Group 1), maximum plasma concentrations (average of 179±54.9 ng/mL) of nicotine-n-oxide metabolite were observed between 30 minutes and 1 hour post dosing. The average half-life after oral dosing could not be determined. The average exposure for nicotine-n-oxide (Group 1) based on the dose normalized AUClast was 11.2±3.32 hr*kg*ng/mL/mg.

Following PO dosing of Reference Nicotine Polacrilex at 10 mg/kg (Group 1), maximum plasma concentrations (average of 193±58.6 ng/mL) of cotinine metabolite were observed at 1 hour post dosing. The average half-life after oral dosing could not be determined. The average exposure for cotinine (Group 1) based on the dose normalized AUClast was 10.9±2.90 hr*kg*ng/mL/mg.

Following PO dosing of Reference Nicotine Polacrilex at 10 mg/kg (Group 2), maximum plasma concentrations (average of 350±256 ng/mL) of nicotine were observed between 8 minutes and 1 hour post dosing. The average half-life after oral dosing could not be determined. The average exposure for nicotine (Group 2) based on the dose normalized AUClast was 21.3±13.7 hr*kg*ng/mL/mg.

Following PO dosing of Reference Nicotine Polacrilex at 10 mg/kg (Group 2), maximum plasma concentrations (average of 20.1±13.3 ng/mL) of hydroxycotinine metabolite were observed at 1 hour post dosing. The average half-life after oral dosing could not be determined. The average exposure for hydroxycotinine (Group 2) based on the dose normalized AUClast was 1.15±0.928 hr*kg*ng/mL/mg.

Following PO dosing of Reference Nicotine Polacrilex at 10 mg/kg (Group 2), maximum plasma concentrations (average of 409±235 ng/mL) of nicotine-n-oxide metabolite were observed between 12 minutes and 1 hour post dosing. The average half-life after oral dosing could not be determined. The average exposure for nicotine-n-oxide (Group 2) based on the dose normalized AUClast was 26.8±18.3 hr*kg*ng/mL/mg.

Following PO dosing of Reference Nicotine Polacrilex at 10 mg/kg (Group 2), maximum plasma concentrations (average of 359±236 ng/mL) of cotinine metabolite were observed between 45 minutes and 1 hour post dosing. The average half-life after oral dosing could not be determined. The average exposure for cotinine (Group 2) based on the dose normalized AUClast was 22.5±16.7 hr*kg*ng/mL/mg.

Following PO dosing of Reference Nicotine Polacrilex at 10 mg/kg (Group 3), maximum plasma concentrations (average of 176±71.2 ng/mL) of nicotine were observed between 30 minutes and 1 hour post dosing. The average half-life after oral dosing could not be determined. The average exposure for nicotine (Group 3) based on the dose normalized AUClast was 11.7±4.62 hr*kg*ng/mL/mg. On average, 1.04±0.49% and 0.03±0.04% of the dose (unchanged dose) was found in urine and feces, respectively, after PO dosing.

Following PO dosing of Reference Nicotine Polacrilex at 10 mg/kg (Group 3), maximum plasma concentrations (average of 13.4±5.95 ng/mL) of hydroxycotinine metabolite were observed between 45 minutes 1 hour post dosing. The average half-life after oral dosing could not be determined. The average exposure for hydroxycotinine (Group 3) based on the dose normalized AUClast was 0.672±0.386 hr*kg*ng/mL/mg. On average, 1.10±0.64% and 0.03% (n=1) of the dose was found in urine and feces, respectively, after PO dosing.

Following PO dosing of Reference Nicotine Polacrilex at 10 mg/kg (Group 3), maximum plasma concentrations (average of 283±134 ng/mL) of nicotine-n-oxide metabolite were observed between 30 minutes and 1 hour post dosing. The average half-life after oral dosing could not be determined. The average exposure for nicotine-n-oxide (Group 3) based on the dose normalized AUClast was 17.8±7.29 hr*kg*ng/mL/mg. On average, 9.36±4.36% and 0.07% (n=1) of the dose was found in urine and feces, respectively, after PO dosing.

Following PO dosing of Reference Nicotine Polacrilex at 10 mg/kg (Group 3), maximum plasma concentrations (average of 304±103 ng/mL) of cotinine metabolite were observed at 1 hour post dosing. The average half-life after oral dosing could not be determined. The average exposure for cotinine (Group 3) based on the dose normalized AUClast was 15.4±4.99 hr*kg*ng/mL/mg. On average, 0.99±0.48% and 0.03±0.02% of the dose was found in urine and feces, respectively, after PO dosing.

Following PO dosing of Reference Nicotine Polacrilex at 10 mg/kg (Group 4), maximum plasma concentrations (average of 210±68.6 ng/mL) of nicotine were observed between 15 minutes and 1 hour post dosing. The average half-life after oral dosing was 0.949±0.214 hours. The average exposure for nicotine (Group 4) based on the dose normalized AUClast was 13.0±4.98 hr*kg*ng/mL/mg. On average, 3.31±0.91% and 0.09±0.07% of the dose (unchanged dose) was found in urine and feces, respectively, after PO dosing.

Following PO dosing of Reference Nicotine Polacrilex at 10 mg/kg (Group 4), maximum plasma concentrations (average of 14.3±4.74 ng/mL) of hydroxycotinine metabolite were observed between 45 minutes and 1 hour post dosing. The average half-life after oral dosing could not be determined. The average exposure for hydroxycotinine (Group 4) based on the dose normalized AUClast was 0.751±0.389 hr*kg*ng/mL/mg. On average, 6.48±2.12% and 0.03±0.02% of the dose was found in urine and feces, respectively, after PO dosing.

Following PO dosing of Reference Nicotine Polacrilex at 10 mg/kg (Group 4), maximum plasma concentrations (average of 223±71.9 ng/mL) of nicotine-n-oxide metabolite were observed between 15 minutes and 1 hour post dosing. The average half-life after oral dosing was 1.38 hours. The average exposure for nicotine-n-oxide (Group 4) based on the dose normalized AUClast was 15.0±6.27 hr*kg*ng/mL/mg. On average, 20.3±6.90% of the dose was found in urine after PO dosing. All concentrations in feces were below the limit of quantitation.

Following PO dosing of Reference Nicotine Polacrilex at 10 mg/kg (Group 4), maximum plasma concentrations (average of 247±49.4 ng/mL) of cotinine metabolite were observed between 45 minutes and 1 hour post dosing. The average half-life after oral dosing could not be determined. The average exposure for cotinine (Group 4) based on the dose normalized AUClast was 14.0±2.60 hr*kg*ng/mL/mg. On average, 5.30±2.18% and 0.16±0.08% of the dose was found in urine and feces, respectively, after PO dosing.

Following PO dosing of Reference Nicotine Polacrilex at 10 mg/kg (Groups 1-4), the average (±SE) Cmax for nicotine in brain tissue was 427±66.5 ng/g, the tmax was 4 hours, the half-life could not be determined, and the exposure for nicotine based on the dose normalized AUClast was 588±53.8 hr*kg*ng/g/mg. After PO dosing of Reference Nicotine Polacrilex, the average (±SE) Cmax for hydroxycotinine metabolite in brain tissue was 51.8±9.14 ng/g, the tmax was 8 hours, the half-life could not be determined, and the exposure for hydroxycotinine metabolite based on the dose normalized AUClast was 95.5±12.1 hr*kg*ng/g/mg. After PO dosing of Reference Nicotine Polacrilex, the majority of the concentrations were below the limit of quantitation and therefore, the pharmacokinetic parameters were not able to be calculated. After PO dosing of Reference Nicotine Polacrilex, the average (±SE) Cmax for cotinine metabolite in brain tissue was 722±135 ng/g, the tmax was 8 hours, the half-life could not be determined, and the exposure for cotinine metabolite based on the dose normalized AUClast was 1332±208 hr*kg*ng/g/mg.

Following PO dosing of Reference Nicotine Polacrilex at 10 mg/kg (Groups 1-4), the average (±SE) Cmax for nicotine in liver tissue was 1300±308 ng/g, the tmax was 4 hours, the half-life could not be determined, and the exposure for nicotine based on the dose normalized AUClast was 1737±167 hr*kg*ng/g/mg. After PO dosing of Reference Nicotine Polacrilex, the average (±SE) Cmax for hydroxycotinine metabolite in liver tissue was 102±13.5 ng/g, the tmax was 8 hours, the half-life could not be determined, and the exposure for hydroxycotinine metabolite based on the dose normalized AUClast was 205±26.3 hr*kg*ng/g/mg. After PO dosing of Reference Nicotine Polacrilex, the average (±SE) Cmax for nicotine-n-oxide metabolite in liver tissue was 4.51±1.58 ng/g, the tmax was 8 hours, the half-life could not be determined, and the exposure for nicotine-n-oxide metabolite based on the dose normalized AUClast was 6.86±1.83 hr*kg*ng/g/mg. After PO dosing of Reference Nicotine Polacrilex, the average (±SE) Cmax for cotinine metabolite in liver tissue was 905±119 ng/g, the tmax was 8 hours, the half-life could not be determined, and the exposure for cotinine metabolite based on the dose normalized AUClast was 1620±189 hr*kg*ng/g/mg.

Following PO dosing of Reference Nicotine Polacrilex at 10 mg/kg (Groups 1-4), the average (±SE) Cmax for nicotine in kidney tissue was 8965±1519 ng/g, the tmax was 4 hours, the half-life could not be determined, and the exposure for nicotine based on the dose normalized AUClast was 12267±1173 hr*kg*ng/g/mg. After PO dosing of Reference Nicotine Polacrilex, the average (±SE) Cmax for hydroxycotinine metabolite in kidney tissue was 200±44.1 ng/g, the tmax was 24 hours, the half-life could not be determined, and the exposure for hydroxycotinine metabolite based on the dose normalized AUClast was 391±47.7 hr*kg*ng/g/mg. After PO dosing of Reference Nicotine Polacrilex, the average (±SE) Cmax for nicotine-n-oxide metabolite in kidney tissue was 20.5±4.26 ng/g, the tmax was 4 hours, the half-life could not be determined, and the exposure for nicotine-n-oxide metabolite based on the dose normalized AUClast was 23.4±2.80 hr*kg*ng/g/mg. After PO dosing of Reference Nicotine Polacrilex, the average (SE) Cmax for cotinine metabolite in kidney tissue was 1775±217 ng/g, the tmax was 8 hours, the half-life could not be determined, and the exposure for cotinine metabolite based on the dose normalized AUClast was 3436±374 hr*kg*ng/g/mg.

Following PO dosing of Test Nicotine Polacrilex at 10 mg/kg (Group 5), maximum plasma concentrations (average of 416±255 ng/mL) of nicotine were observed between 12 minutes and 1 hour post dosing. The average half-life after oral dosing could not be determined. The average exposure for nicotine (Group 5) based on the dose normalized AUClast was 28.7±13.8 hr*kg*ng/mL/mg.

Following PO dosing of Test Nicotine Polacrilex at 10 mg/kg (Group 5), maximum plasma concentrations (average of 13.9±3.07 ng/mL) of hydroxycotinine metabolite were observed at 1 hour post dosing. The average half-life after oral dosing could not be determined. The average exposure for hydroxycotinine (Group 5) based on the dose normalized AUClast was 0.671±0.167 hr*kg*ng/mL/mg.

Following PO dosing of Test Nicotine Polacrilex at 10 mg/kg (Group 5), maximum plasma concentrations (average of 267±56.1 ng/mL) of nicotine-n-oxide metabolite were observed between 45 minutes and 1 hour post dosing. The average half-life after oral dosing could not be determined. The average exposure for nicotine-n-oxide (Group 5) based on the dose normalized AUClast was 19.3±3.45 hr*kg*ng/mL/mg.

Following PO dosing of Test Nicotine Polacrilex at 10 mg/kg (Group 5), maximum plasma concentrations (average of 381±81.8 ng/mL) of cotinine metabolite were observed at 1 hour post dosing. The average half-life after oral dosing could not be determined. The average exposure for cotinine (Group 5) based on the dose normalized AUClast was 21.3±5.76 hr*kg*ng/mL/mg.

Following PO dosing of Test Nicotine Polacrilex at 10 mg/kg (Group 6), maximum plasma concentrations (average of 315±142 ng/mL) of nicotine were observed between 15 minutes and 1 hour post dosing. The average half-life after oral dosing could not be determined. The average exposure for nicotine (Group 6) based on the dose normalized AUClast was 21.5±10.8 hr*kg*ng/mL/mg.

Following PO dosing of Test Nicotine Polacrilex at 10 mg/kg (Group 6), maximum plasma concentrations (average of 11.6±2.62 ng/mL) of hydroxycotinine metabolite were observed between 45 minutes and 1 hour post dosing. The average half-life after oral dosing could not be determined. The average exposure for hydroxycotinine (Group 6) based on the dose normalized AUClast was 0.581±0.149 hr*kg*ng/mL/mg.

Following PO dosing of Test Nicotine Polacrilex at 10 mg/kg (Group 6), maximum plasma concentrations (average of 246±120 ng/mL) of nicotine-n-oxide metabolite were observed between 15 minutes and 1 hour post dosing. The average half-life after oral dosing could not be determined. The average exposure for nicotine-n-oxide (Group 6) based on the dose normalized AUClast was 15.6±8.37 hr*kg*ng/mL/mg.

Following PO dosing of Test Nicotine Polacrilex at 10 mg/kg (Group 6), maximum plasma concentrations (average of 315±76.8 ng/mL) of cotinine metabolite were observed between 45 minutes and 1 hour post dosing. The average half-life after oral dosing could not be determined. The average exposure for cotinine (Group 6) based on the dose normalized AUClast was 17.7±5.25 hr*kg*ng/mL/mg.

Following PO dosing of Test Nicotine Polacrilex at 10 mg/kg (Group 7), maximum plasma concentrations (average of 253±40.0 ng/mL) of nicotine were observed between 12 minutes and 1 hour post dosing. The average half-life after oral dosing could not be determined. The average exposure for nicotine (Group 7) based on the dose normalized AUClast was 18.3±6.21 hr*kg*ng/mL/mg. On average, 2.02±1.21% and 0.04±0.04% of the dose (unchanged dose) was found in urine and feces, respectively, after PO dosing.

Following PO dosing of Test Nicotine Polacrilex at 10 mg/kg (Group 7), maximum plasma concentrations (average of 12.7±4.62 ng/mL) of hydroxycotinine metabolite were observed at 1 hour post dosing. The average half-life after oral dosing could not be determined. The average exposure for hydroxycotinine (Group 7) based on the dose normalized AUClast was 0.620±0.253 hr*kg*ng/mL/mg. On average, 0.97±0.34% and 0.02% (n=1) of the dose was found in urine and feces, respectively, after PO dosing.

Following PO dosing of Test Nicotine Polacrilex at 10 mg/kg (Group 7), maximum plasma concentrations (average of 276±67.5 ng/mL) of nicotine-n-oxide metabolite were observed between 15 minutes and 1 hour post dosing. The average half-life after oral dosing was 2.84 hours. The average exposure for nicotine-n-oxide (Group 7) based on the dose normalized AUClast was 17.6±6.17 hr*kg*ng/mL/mg. On average, 9.91±4.61% and 0.12% of the dose was found in urine and feces, respectively, after PO dosing.

Following PO dosing of Test Nicotine Polacrilex at 10 mg/kg (Group 7), maximum plasma concentrations (average of 317±100 ng/mL) of cotinine metabolite were observed at 1 hour post dosing. The average half-life after oral dosing could not be determined. The average exposure for cotinine (Group 7) based on the dose normalized AUClast was 16.6±4.69 hr*kg*ng/mL/mg. On average, 1.39±0.80% and 0.02±0.01% of the dose was found in urine and feces, respectively, after PO dosing.

Following PO dosing of Test Nicotine Polacrilex at 10 mg/kg (Group 8), maximum plasma concentrations (average of 593±641 ng/mL) of nicotine were observed between 8 minutes and 1 hour post dosing. The average half-life after oral dosing could not be determined; however, the half-life for one rat was 0.737 hours. The average exposure for nicotine (Group 8) based on the dose normalized AUClast was 38.0±38.5 hr*kg*ng/mL/mg. On average, 5.91±3.24% and 0.06±0.03% of the dose (unchanged dose) was found in urine and feces, respectively, after PO dosing.

Following PO dosing of Test Nicotine Polacrilex at 10 mg/kg (Group 8), maximum plasma concentrations (average of 17.4±13.8 ng/mL) of hydroxycotinine metabolite were observed between 45 minutes and 1 hour post dosing. The average half-life after oral dosing could not be determined. The average exposure for hydroxycotinine (Group 8) based on the dose normalized AUClast was 0.940±0.788 hr*kg*ng/mL/mg. On average, 9.07±3.61% and 0.02±0.01% of the dose was found in urine and feces, respectively, after PO dosing.

Following PO dosing of Test Nicotine Polacrilex at 10 mg/kg (Group 8), maximum plasma concentrations (average of 357±306 ng/mL) of nicotine-n-oxide metabolite were observed between 15 minutes and 1 hour post dosing. The average half-life after oral dosing could not be determined; however, the half-life for one rat was 0.888 hours. The average exposure for nicotine-n-oxide (Group 8) based on the dose normalized AUClast was 27.5±23.8 hr*kg*ng/mL/mg. On average, 39.5±9.71% and 0.08% of the dose was found in urine and feces, respectively, after PO dosing.

Following PO dosing of Test Nicotine Polacrilex at 10 mg/kg (Group 8), maximum plasma concentrations (average of 441±333 ng/mL) of cotinine metabolite were observed at 1 hour post dosing. The average half-life after oral dosing could not be determined. The average exposure for cotinine (Group 8) based on the dose normalized AUClast was 25.8±20.0 hr*kg*ng/mL/mg. On average, 8.23±2.58% and 0.18±0.10% of the dose was found in urine and feces, respectively, after PO dosing.

Following PO dosing of Test Nicotine Polacrilex at 10 mg/kg (Groups 5-8), the average (±SE) Cmax for nicotine in brain tissue was 1260±200 ng/g, the tmax was 1 hour, the half-life was 21.6 hours, and the exposure for nicotine based on the dose normalized AUClast was 1300±125 hr*kg*ng/g/mg. After PO dosing of Test Nicotine Polacrilex, the average (±SE) Cmax for hydroxycotinine metabolite in brain tissue was 91.2±7.69 ng/g, the tmax was 24 hours, the half-life could not be determined, and the exposure for hydroxycotinine metabolite based on the dose normalized AUClast was 142±6.64 hr*kg*ng/g/mg. After PO dosing of Test Nicotine Polacrilex, the average (±SE) Cmax for nicotine-n-oxide metabolite in brain tissue was 4.17±1.41 ng/g, the tmax was 1 hour, the half-life could not be determined, and the exposure for nicotine-n-oxide metabolite based on the dose normalized AUClast was 2.70±1.05 hr*kg*ng/g/mg. After PO dosing of Test Nicotine Polacrilex, the average (±SE) Cmax for cotinine metabolite in brain tissue was 1322±219 ng/g, the tmax was 24 hours, the half-life could not be determined, and the exposure for cotinine metabolite based on the dose normalized AUClast was 2172±189 hr*kg*ng/g/mg.

Following PO dosing of Test Nicotine Polacrilex at 10 mg/kg (Groups 5-8), the average (±SE) Cmax for nicotine in liver tissue was 2702±308 ng/g, the tmax was 1 hour, the half-life was 18.9 hours, and the exposure for nicotine based on the dose normalized AUClast was 2989±277 hr*kg*ng/g/mg. After PO dosing of Test Nicotine Polacrilex, the average (±SE) Cmax for hydroxycotinine metabolite in liver tissue was 232±41.2 ng/g, the tmax was 24 hours, the half-life could not be determined, and the exposure for hydroxycotinine metabolite based on the dose normalized AUClast was 338±37.6 hr*kg*ng/g/mg. After PO dosing of Test Nicotine Polacrilex, the average (±SE) Cmax for nicotine-n-oxide metabolite in liver tissue was 6.69±1.67 ng/g, the tmax was 1 hour, the half-life could not be determined, and the exposure for nicotine-n-oxide metabolite based on the dose normalized AUClast was 8.74±2.56 hr*kg*ng/g/mg. After PO dosing of Test Nicotine Polacrilex, the average (±SE) Cmax for cotinine metabolite in liver tissue was 1451±157 ng/g, the tmax was 24 hours, the half-life could not be determined, and the exposure for cotinine metabolite based on the dose normalized AUClast was 2505±139 hr*kg*ng/g/mg.

Following PO dosing of Test Nicotine Polacrilex at 10 mg/kg (Groups 5-8), the average (±SE) Cmax for nicotine in kidney tissue was 8930±676 ng/g, the tmax was 1 hour, the half-life was 24.2 hours, and the exposure for nicotine based on the dose normalized AUClast was 12717±1354 hr*kg*ng/g/mg. After PO dosing of Test Nicotine Polacrilex, the average (±SE) Cmax for hydroxycotinine metabolite in kidney tissue was 244±16.5 ng/g, the tmax was 24 hours, the half-life could not be determined, and the exposure for hydroxycotinine metabolite based on the dose normalized AUClast was 449±24.1 hr*kg*ng/g/mg. After PO dosing of Test Nicotine Polacrilex, the average (+SE) Cmax for nicotine-n-oxide metabolite in kidney tissue was 28.0±6.34 ng/g, the tmax was 1 hour, the half-life could not be determined, and the exposure for nicotine-n-oxide metabolite based on the dose normalized AUClast was 38.0±5.57 hr*kg*ng/g/mg. After PO dosing of Test Nicotine Polacrilex, the average (±SE) Cmax for cotinine metabolite in kidney tissue was 2466±321 ng/g, the tmax was 24 hours, the half-life could not be determined, and the exposure for cotinine metabolite based on the dose normalized AUClast was 4300±280 hr*kg*ng/g/mg.

### Example 6

In this study, the exposure and distribution of nicotine and its major metabolites were evaluated following oral administration of two separate formulations (Reference and Test Nicotine Polacrilex) in male Sprague-Dawley rats. Formulations were administered orally (PO) at 10 mg/kg. Following dosing, blood samples were collected up to 1 hour post dose; and urine and fecal samples were collected up to 24 hours post dose. Brain, liver, and kidney tissue were collected at 1 hour(Groups 1 & 5), 4 hours (Groups 2 & 6), following the 8 hour urine and feces sample collection (Groups 3 & 7), or following the 24 hour urine and feces sample collection (Groups 4 & 8). Blood, urine, feces, and tissue concentrations of each analyte were determined by LC-MS/MS. Plasma pharmacokinetic parameters were determined using WinNonlin (v8.0). Brain, liver, and kidney pharmacokinetic parameters were determined using WinNonlin (v8.0) software with sparse sampling.

Following PO dosing of Reference Nicotine Polacrilex at 10 mg/kg (Group 1), maximum plasma concentrations (average of 144±68.2 ng/mL) of nicotine were observed between 30 minutes and 1 hourpost dosing. The average half-life after oral dosing could not be determined. The average exposure for nicotine (Group 1) based on the dose normalized AUClast was 8.71±2.76 hr*kg*ng/mL/mg.

Following PO dosing of Reference Nicotine Polacrilex at 10 mg/kg (Group 1), maximum plasma concentrations (average of 9.79±3.56 ng/mL) of hydroxycotinine metabolite were observed between 45 minutes and 1 hour post dosing. The average half-life after oral dosing could not be determined. The average exposure for hydroxycotinine (Group 1) based on the dose normalized AUClast was 0.420±0.146 hr*kg*ng/mL/mg.

Following PO dosing of Reference Nicotine Polacrilex at 10 mg/kg (Group 1), maximum plasma concentrations (average of 179±54.9 ng/mL) of nicotine-n-oxide metabolite were observed between 30 minutes and 1 hour post dosing. The average half-life after oral dosing could not be determined. The average exposure for nicotine-n-oxide (Group 1) based on the dose normalized AUClast was 11.2±3.32 hr*kg*ng/mL/mg.

Following PO dosing of Reference Nicotine Polacrilex at 10 mg/kg (Group 1), maximum plasma concentrations (average of 193±58.6 ng/mL) of cotinine metabolite were observed at 1 hour post dosing. The average half-life after oral dosing could not be determined. The average exposure for cotinine (Group 1) based on the dose normalized AUClast was 10.9±2.90 hr*kg*ng/mL/mg.

Following PO dosing of Reference Nicotine Polacrilex at 10 mg/kg (Group 2), maximum plasma concentrations (average of 350±256 ng/mL) of nicotine were observed between 8 minutes and 1 hour post dosing. The average half-life after oral dosing could not be determined. The average exposure for nicotine (Group 2) based on the dose normalized AUClast was 21.3±13.7 hr*kg*ng/mL/mg.

Following PO dosing of Reference Nicotine Polacrilex at 10 mg/kg (Group 2), maximum plasma concentrations (average of 20.1±13.3 ng/mL) of hydroxycotinine metabolite were observed at 1 hour post dosing. The average half-life after oral dosing could not be determined. The average exposure for hydroxycotinine (Group 2) based on the dose normalized AUClast was 1.15±0.928 hr*kg*ng/mL/mg.

Following PO dosing of Reference Nicotine Polacrilex at 10 mg/kg (Group 2), maximum plasma concentrations (average of 409±235 ng/mL) of nicotine-n-oxide metabolite were observed between 12 minutes and 1 hour post dosing. The average half-life after oral dosing could not be determined. The average exposure for nicotine-n-oxide (Group 2) based on the dose normalized AUClast was 26.8±18.3 hr*kg*ng/mL/mg.

Following PO dosing of Reference Nicotine Polacrilex at 10 mg/kg (Group 2), maximum plasma concentrations (average of 359±236 ng/mL) of cotinine metabolite were observed between 45 minutes and 1 hour post dosing. The average half-life after oral dosing could not be determined. The average exposure for cotinine (Group 2) based on the dose normalized AUClast was 22.5±16.7 hr*kg*ng/mL/mg.

Following PO dosing of Reference Nicotine Polacrilex at 10 mg/kg (Group 3), maximum plasma concentrations (average of 176±71.2 ng/mL) of nicotine were observed between 30 minutes and 1 hour post dosing. The average half-life after oral dosing could not be determined. The average exposure for nicotine (Group 3) based on the dose normalized AUClast was 11.7±4.62 hr*kg*ng/mL/mg. On average, 1.04±0.49% and 0.03±0.04% of the dose (unchanged dose) was found in urine and feces, respectively, after PO dosing.

Following PO dosing of Reference Nicotine Polacrilex at 10 mg/kg (Group 3), maximum plasma concentrations (average of 13.4±5.95 ng/mL) of hydroxycotinine metabolite were observed between 45 minutes 1 hour post dosing. The average half-life after oral dosing could not be determined. The average exposure for hydroxycotinine (Group 3) based on the dose normalized AUClast was 0.672±0.386 hr*kg*ng/mL/mg. On average, 1.10±0.64% and 0.03% (n=1) of the dose was found in urine and feces, respectively, after PO dosing.

Following PO dosing of Reference Nicotine Polacrilex at 10 mg/kg (Group 3), maximum plasma concentrations (average of 283±134 ng/mL) of nicotine-n-oxide metabolite were observed between 30 minutes and 1 hour post dosing. The average half-life after oral dosing could not be determined. The average exposure for nicotine-n-oxide (Group 3) based on the dose normalized AUClast was 17.8±7.29 hr*kg*ng/mL/mg. On average, 9.36±4.36% and 0.07% (n=1) of the dose was found in urine and feces, respectively, after PO dosing.

Following PO dosing of Reference Nicotine Polacrilex at 10 mg/kg (Group 3), maximum plasma concentrations (average of 304±103 ng/mL) of cotinine metabolite were observed at 1 hourpost dosing. The average half-life after oral dosing could not be determined. The average exposure for cotinine (Group 3) based on the dose normalized AUClast was 15.4±4.99 hr*kg*ng/mL/mg. On average, 0.99±0.48% and 0.03±0.02% of the dose was found in urine and feces, respectively, after PO dosing.

Following PO dosing of Reference Nicotine Polacrilex at 10 mg/kg (Group 4), maximum plasma concentrations (average of 210±68.6 ng/mL) of nicotine were observed between 15 minutes and 1 hour post dosing. The average half-life after oral dosing was 0.949±0.214 hours. The average exposure for nicotine (Group 4) based on the dose normalized AUClast was 13.0±4.98 hr*kg*ng/mL/mg. On average, 3.31±0.91% and 0.09±0.07% of the dose (unchanged dose) was found in urine and feces, respectively, after PO dosing.

Following PO dosing of Reference Nicotine Polacrilex at 10 mg/kg (Group 4), maximum plasma concentrations (average of 14.3±4.74 ng/mL) of hydroxycotinine metabolite were observed between 45 minutes and 1 hour post dosing. The average half-life after oral dosing could not be determined. The average exposure for hydroxycotinine (Group 4) based on the dose normalized AUClast was 0.751±0.389 hr*kg*ng/mL/mg. On average, 6.48±2.12% and 0.03±0.02% of the dose was found in urine and feces, respectively, after PO dosing.

Following PO dosing of Reference Nicotine Polacrilex at 10 mg/kg (Group 4), maximum plasma concentrations (average of 223±71.9 ng/mL) of nicotine-n-oxide metabolite were observed between 15 minutes and 1 hour post dosing. The average half-life after oral dosing was 1.38 hours. The average exposure for nicotine-n-oxide (Group 4) based on the dose normalized AUClast was 15.0±6.27 hr*kg*ng/mL/mg. On average, 20.3±6.90% of the dose was found in urine after PO dosing. All concentrations in feces were below the limit of quantitation.

Following PO dosing of Reference Nicotine Polacrilex at 10 mg/kg (Group 4), maximum plasma concentrations (average of 247±49.4 ng/mL) of cotinine metabolite were observed between 45 minutes and 1 hour post dosing. The average half-life after oral dosing could not be determined. The average exposure for cotinine (Group 4) based on the dose normalized AUClast was 14.0±2.60 hr*kg*ng/mL/mg. On average, 5.30±2.18% and 0.16±0.08% of the dose was found in urine and feces, respectively, after PO dosing.

Following PO dosing of Reference Nicotine Polacrilex at 10 mg/kg (Groups 1-4), the average (±SE) Cmax for nicotine in brain tissue was 427±66.5 ng/g, the tmax was 4 hours, the half-life could not be determined, and the exposure for nicotine based on the dose normalized AUClast was 588±53.8 hr*kg*ng/g/mg. After PO dosing of Reference Nicotine Polacrilex, the average (±SE) Cmax for hydroxycotinine metabolite in brain tissue was 51.8±9.14 ng/g, the tmax was 8 hours, the half-life could not be determined, and the exposure for hydroxycotinine metabolite based on the dose normalized AUClast was 95.5±12.1 hr*kg*ng/g/mg. After PO dosing of Reference Nicotine Polacrilex, the majority of the concentrations were below the limit of quantitation and therefore, the pharmacokinetic parameters were not able to be calculated. After PO dosing of Reference Nicotine Polacrilex, the average (±SE) Cmax for cotinine metabolite in brain tissue was 722±135 ng/g, the tmax was 8 hours, the half-life could not be determined, and the exposure for cotinine metabolite based on the dose normalized AUClast was 1332±208 hr*kg*ng/g/mg.

Following PO dosing of Reference Nicotine Polacrilex at 10 mg/kg (Groups 1-4), the average (SE) Cmax for nicotine in liver tissue was 1300±308 ng/g, the tmax was 4 hours, the half-life could not be determined, and the exposure for nicotine based on the dose normalized AUClast was 1737±167 hr*kg*ng/g/mg. After PO dosing of Reference Nicotine Polacrilex, the average (+SE) Cmax for hydroxycotinine metabolite in liver tissue was 102±13.5 ng/g, the tmax was 8 hours, the half-life could not be determined, and the exposure for hydroxycotinine metabolite based on the dose normalized AUClast was 205±26.3 hr*kg*ng/g/mg. After PO dosing of Reference Nicotine Polacrilex, the average (±SE) Cmax for nicotine-n-oxide metabolite in liver tissue was 4.51±1.58 ng/g, the tmax was 8 hours, the half-life could not be determined, and the exposure for nicotine-n-oxide metabolite based on the dose normalized AUClast was 6.86±1.83 hr*kg*ng/g/mg. After PO dosing of Reference Nicotine Polacrilex, the average (±SE) Cmax for cotinine metabolite in liver tissue was 905±119 ng/g, the tmax was 8 hours, the half-life could not be determined, and the exposure for cotinine metabolite based on the dose normalized AUClast was 1620±189 hr*kg*ng/g/mg.

Following PO dosing of Reference Nicotine Polacrilex at 10 mg/kg (Groups 1-4), the average (±SE) Cmax for nicotine in kidney tissue was 8965±1519 ng/g, the tmax was 4 hours, the half-life could not be determined, and the exposure for nicotine based on the dose normalized AUClast was 12267±1173 hr*kg*ng/g/mg. After PO dosing of Reference Nicotine Polacrilex, the average (±SE) Cmax for hydroxycotinine metabolite in kidney tissue was 200±44.1 ng/g, the tmax was 24 hours, the half-life could not be determined, and the exposure for hydroxycotinine metabolite based on the dose normalized AUClast was 391±47.7 hr*kg*ng/g/mg. After PO dosing of Reference Nicotine Polacrilex, the average (+SE) Cmax for nicotine-n-oxide metabolite in kidney tissue was 20.5±4.26 ng/g, the tmax was 4 hours, the half-life could not be determined, and the exposure for nicotine-n-oxide metabolite based on the dose normalized AUClast was 23.4±2.80 hr*kg*ng/g/mg. After PO dosing of Reference Nicotine Polacrilex, the average (SE) Cmax for cotinine metabolite in kidney tissue was 1775±217 ng/g, the tmax was 8 hours, the half life could not be determined, and the exposure for cotinine metabolite based on the dose normalized AUClast was 3436±374 hr*kg*ng/g/mg.

Following PO dosing of Test Nicotine Polacrilex at 10 mg/kg (Group 5), maximum plasma concentrations (average of 416±255 ng/mL) of nicotine were observed between 12 minutes and 1 hourpost dosing. The average half-life after oral dosing could not be determined. The average exposure for nicotine (Group 5) based on the dose normalized AUClast was 28.7±13.8 Following PO dosing of Test Nicotine Polacrilex at 10 mg/kg (Group 5), maximum plasma concentrations (average of 13.9±3.07 ng/mL) of hydroxycotinine metabolite were observed at 1 hour post dosing. The average half-life after oral dosing could not be determined. The average exposure for hydroxycotinine (Group 5) based on the dose normalized AUClast was 0.671±0.167 hr*kg*ng/mL/mg.

Following PO dosing of Test Nicotine Polacrilex at 10 mg/kg (Group 5), maximum plasma concentrations (average of 267±56.1 ng/mL) of nicotine-n-oxide metabolite were observed between 45 minutes and 1 hour post dosing. The average half-life after oral dosing could not be determined. The average exposure for nicotine-n-oxide (Group 5) based on the dose normalized AUClast was 19.3±3.45 hr*kg*ng/mL/mg.

Following PO dosing of Test Nicotine Polacrilex at 10 mg/kg (Group 5), maximum plasma concentrations (average of 381±81.8 ng/mL) of cotinine metabolite were observed at 1 hour post dosing. The average half-life after oral dosing could not be determined. The average exposure for cotinine (Group 5) based on the dose normalized AUClast was 21.3±5.76 hr*kg*ng/mL/mg.

Following PO dosing of Test Nicotine Polacrilex at 10 mg/kg (Group 6), maximum plasma concentrations (average of 315±142 ng/mL) of nicotine were observed between 15 minutes and 1 hourpost dosing. The average half-life after oral dosing could not be determined. The average exposure for nicotine (Group 6) based on the dose normalized AUClast was 21.5±10.8 hr*kg*ng/mL/mg.

Following PO dosing of Test Nicotine Polacrilex at 10 mg/kg (Group 6), maximum plasma concentrations (average of 11.6±2.62 ng/mL) of hydroxycotinine metabolite were observed between 45 minutes and 1 hour post dosing. The average half-life after oral dosing could not be determined. The average exposure for hydroxycotinine (Group 6) based on the dose normalized AUClast was 0.581±0.149 hr*kg*ng/mL/mg.

Following PO dosing of Test Nicotine Polacrilex at 10 mg/kg (Group 6), maximum plasma concentrations (average of 246±120 ng/mL) of nicotine-n-oxide metabolite were observed between 15 minutes and 1 hour post dosing. The average half-life after oral dosing could not be determined. The average exposure for nicotine-n-oxide (Group 6) based on the dose normalized AUClast was 15.6±8.37 hr*kg*ng/mL/mg.

Following PO dosing of Test Nicotine Polacrilex at 10 mg/kg (Group 6), maximum plasma concentrations (average of 315±76.8 ng/mL) of cotinine metabolite were observed between 45 minutes and 1 hourpost dosing. The average half-life after oral dosing could not be determined. The average exposure for cotinine (Group 6) based on the dose normalized AUClast was 17.7±5.25 hr*kg*ng/mL/mg. Following PO dosing of Test Nicotine Polacrilex at 10 mg/kg (Group 7), maximum plasma concentrations (average of 253±40.0 ng/mL) of nicotine were observed between 12 minutes and 1 hour post dosing. The average half-life after oral dosing could not be determined. The average exposure for nicotine (Group 7) based on the dose normalized AUClast was 18.3±6.21 hr*kg*ng/mL/mg. On average, 2.02±1.21% and 0.04±0.04% of the dose (unchanged dose) was found in urine and feces, respectively, after PO dosing.

Following PO dosing of Test Nicotine Polacrilex at 10 mg/kg (Group 7), maximum plasma concentrations (average of 12.7±4.62 ng/mL) of hydroxycotinine metabolite were observed at 1 hour post dosing. The average half-life after oral dosing could not be determined. The average exposure for hydroxycotinine (Group 7) based on the dose normalized AUClast was 0.620±0.253 hr*kg*ng/mL/mg. On average, 0.97±0.34% and 0.02% (n=1) of the dose was found in urine and feces, respectively, after PO dosing.

Following PO dosing of Test Nicotine Polacrilex at 10 mg/kg (Group 7), maximum plasma concentrations (average of 276±67.5 ng/mL) of nicotine-n-oxide metabolite were observed between 15 minutes and 1 hour post dosing. The average half-life after oral dosing was 2.84 hours. The average exposure for nicotine-n-oxide (Group 7) based on the dose normalized AUClast was 17.6±6.17 hr*kg*ng/mL/mg. On average, 9.91±4.61% and 0.12% of the dose was found in urine and feces, respectively, after PO dosing.

Following PO dosing of Test Nicotine Polacrilex at 10 mg/kg (Group 7), maximum plasma concentrations (average of 317±100 ng/mL) of cotinine metabolite were observed at 1 hourpost dosing. The average half-life after oral dosing could not be determined. The average exposure for cotinine (Group 7) based on the dose normalized AUClast was 16.6±4.69 hr*kg*ng/mL/mg. On average, 1.39±0.80% and 0.02±0.01% of the dose was found in urine and feces, respectively, after PO dosing.

Following PO dosing of Test Nicotine Polacrilex at 10 mg/kg (Group 8), maximum plasma concentrations (average of 593±641 ng/mL) of nicotine were observed between 8 minutes and 1 hour post dosing. The average half-life after oral dosing could not be determined; however, the half-life for one rat was 0.737 hours. The average exposure for nicotine (Group 8) based on the dose normalized AUClast was 38.0±38.5 hr*kg*ng/mL/mg. On average, 5.91±3.24% and 0.06±0.03% of the dose (unchanged dose) was found in urine and feces, respectively, after PO dosing.

Following PO dosing of Test Nicotine Polacrilex at 10 mg/kg (Group 8), maximum plasma concentrations (average of 17.4±13.8 ng/mL) of hydroxycotinine metabolite were observed between 45 minutes and 1 hour post dosing. The average half-life after oral dosing could not be determined. The average exposure for hydroxycotinine (Group 8) based on the dose normalized

AUClast was 0.940±0.788 hr*kg*ng/mL/mg. On average, 9.07±3.61% and 0.02±0.01% of the dose was found in urine and feces, respectively, after PO dosing.

Following PO dosing of Test Nicotine Polacrilex at 10 mg/kg (Group 8), maximum plasma concentrations (average of 357±306 ng/mL) of nicotine-n-oxide metabolite were observed between 15 minutes and 1 hour post dosing. The average half-life after oral dosing could not be determined; however, the half-life for one rat was 0.888 hours. The average exposure for nicotine-n-oxide (Group 8) based on the dose normalized AUClast was 27.5±23.8 hr*kg*ng/mL/mg. On average, 39.5±9.71% and 0.08% of the dose was found in urine and feces, respectively, after PO dosing.

Following PO dosing of Test Nicotine Polacrilex at 10 mg/kg (Group 8), maximum plasma concentrations (average of 441±333 ng/mL) of cotinine metabolite were observed at 1 hourpost dosing. The average half-life after oral dosing could not be determined. The average exposure for cotinine (Group 8) based on the dose normalized AUClast was 25.8±20.0 hr*kg*ng/mL/mg. On average, 8.23±2.58% and 0.18±0.10% of the dose was found in urine and feces, respectively, after PO dosing.

Following PO dosing of Test Nicotine Polacrilex at 10 mg/kg (Groups 5-8), the average (±SE) Cmax for nicotine in brain tissue was 1260±200 ng/g, the tmax was 1 hour, the half-life was 21.6 hours, and the exposure for nicotine based on the dose normalized AUClast was 1300±125 hr*kg*ng/g/mg. After PO dosing of Test Nicotine Polacrilex, the average (±SE) Cmax for hydroxycotinine metabolite in brain tissue was 91.2±7.69 ng/g, the tmax was 24 hours, the half life could not be determined, and the exposure for hydroxycotinine metabolite based on the dose normalized AUClast was 142±6.64 hr*kg*ng/g/mg. After PO dosing of Test Nicotine Polacrilex, the average (±SE) Cmax for nicotine-n-oxide metabolite in brain tissue was 4.17±1.41 ng/g, the tmax was 1 hour, the half-life could not be determined, and the exposure for nicotine-n-oxide metabolite based on the dose normalized AUClast was 2.70±1.05 hr*kg*ng/g/mg. After PO dosing of Test Nicotine Polacrilex, the average (±SE) Cmax for cotinine metabolite in brain tissue was 1322±219 ng/g, the tmax was 24 hours, the half-life could not be determined, and the exposure for cotinine metabolite based on the dose normalized AUClast was 2172±189 hr*kg*ng/g/mg.

Following PO dosing of Test Nicotine Polacrilex at 10 mg/kg (Groups 5-8), the average (±SE) Cmax for nicotine in liver tissue was 2702±308 ng/g, the tmax was 1 hour, the half-life was 18.9 hours, and the exposure for nicotine based on the dose normalized AUClast was 2989±277 hr*kg*ng/g/mg. After PO dosing of Test Nicotine Polacrilex, the average (±SE) Cmax for hydroxycotinine metabolite in liver tissue was 232±41.2 ng/g, the tmax was 24 hours, the half life could not be determined, and the exposure for hydroxycotinine metabolite based on the dose normalized AUClast was 338±37.6 hr*kg*ng/g/mg. After PO dosing of Test Nicotine Polacrilex, the average (±SE) Cmax for nicotine-n-oxide metabolite in liver tissue was 6.69±1.67 ng/g, the tmax was 1 hour, the half-life could not be determined, and the exposure for nicotine-n-oxide metabolite based on the dose normalized AUClast was 8.74±2.56 hr*kg*ng/g/mg. After PO dosing of Test Nicotine Polacrilex, the average (+SE) Cmax for cotinine metabolite in liver tissue was 1451±157 ng/g, the tmax was 24 hours, the half-life could not be determined, and the exposure for cotinine metabolite based on the dose normalized AUClast was 2505±139 hr*kg*ng/g/mg.

Following PO dosing of Test Nicotine Polacrilex at 10 mg/kg (Groups 5-8), the average (+SE) Cmax for nicotine in kidney tissue was 8930±676 ng/g, the tmax was 1 hour, the half-life was 24.2 hours, and the exposure for nicotine based on the dose normalized AUClast was 12717±1354 hr*kg*ng/g/mg. After PO dosing of Test Nicotine Polacrilex, the average (+SE) Cmax for hydroxycotinine metabolite in kidney tissue was 244±16.5 ng/g, the tmax was 24 hours, the half life could not be determined, and the exposure for hydroxycotinine metabolite based on the dose normalized AUClast was 449±24.1 hr*kg*ng/g/mg. After PO dosing of Test Nicotine Polacrilex, the average (+SE) Cmax for nicotine-n-oxide metabolite in kidney tissue was 28.0±6.34 ng/g, the tmax was 1 hour, the half-life could not be determined, and the exposure for nicotine-n-oxide metabolite based on the dose normalized AUClast was 38.0±5.57 hr*kg*ng/g/mg. After PO dosing of Test Nicotine Polacrilex, the average (+SE) Cmax for cotinine metabolite in kidney tissue was 2466±321 ng/g, the tmax was 24 hours, the half-life could not be determined, and the exposure for cotinine metabolite based on the dose normalized AUClast was 4300280 hr*kg*ng/g/mg.

### Example 7

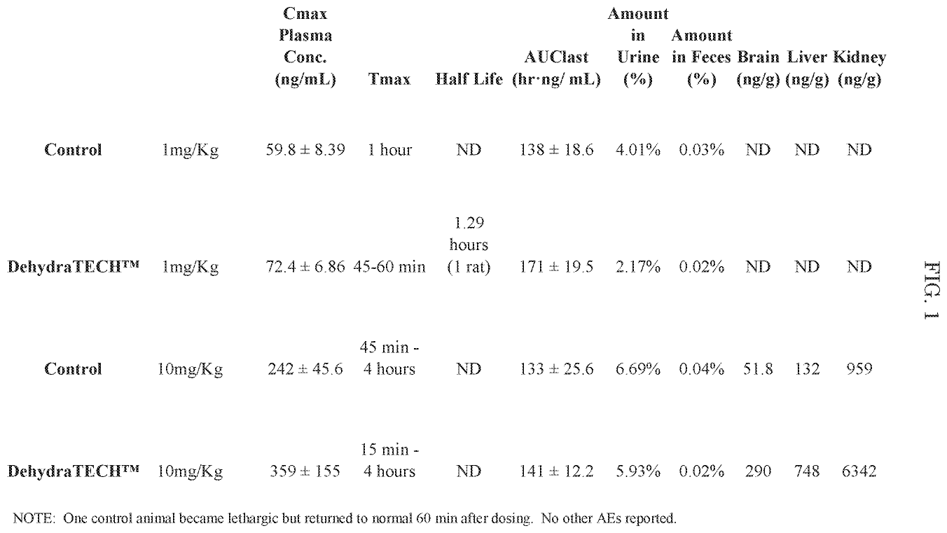

Pharmacokinetic (PK) results from Examples 5 and 6 were compared. FIG. 1 shows PK results from Example 5 comparing nicotine concentrations in various tissues following administration of DEHYDRATECH™ and control compositions in rats.

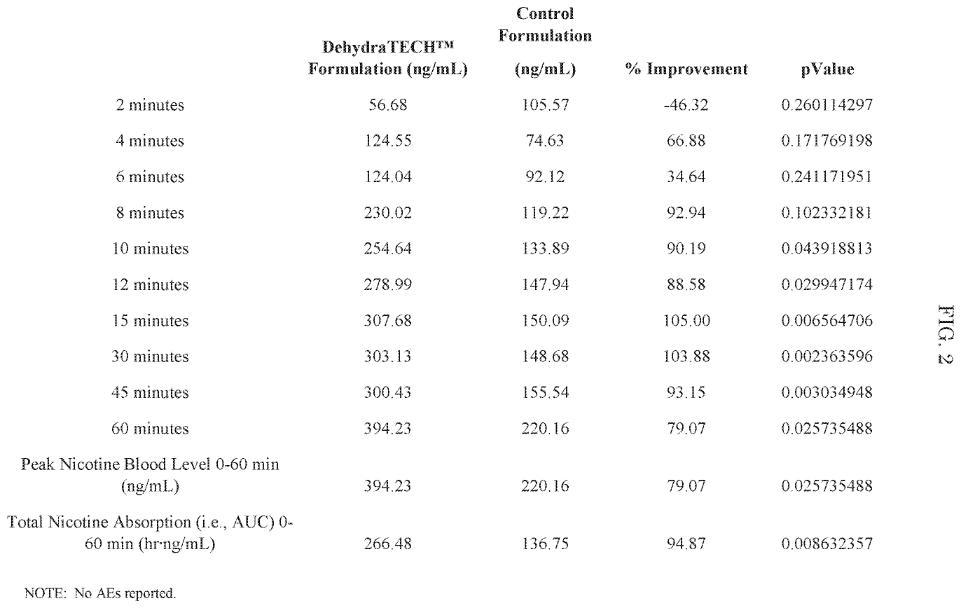

FIG. 2 shows results from Example 6 showing improvement in peak nicotine blood levels following administration of DEHYDRATECH™ and control compositions in rats. A significant improvement in the DEHYDRATECH™ compared to control formulation was observed by 10 minutes after administration.

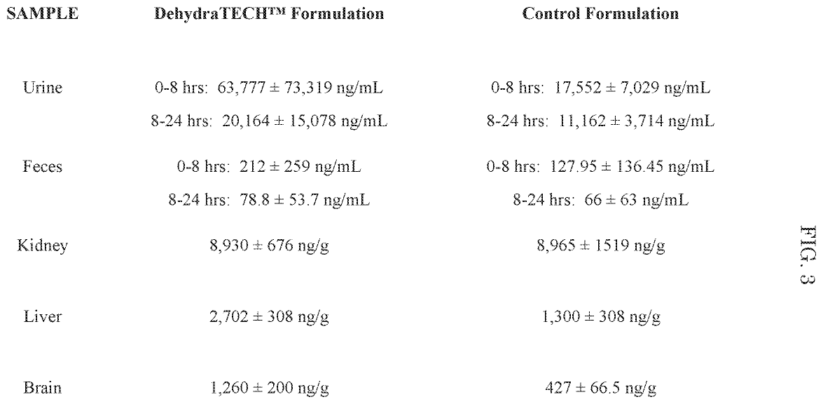

FIG. 3 shows results from Example 6 comparing nicotine concentrations in various tissues following administration of DEHYDRATECH™ and control compositions in rats. A significantly greater concentration of nicotine was observed in brain tissue in the DEHYDRATECH™ treated animals compared to the control formulation.

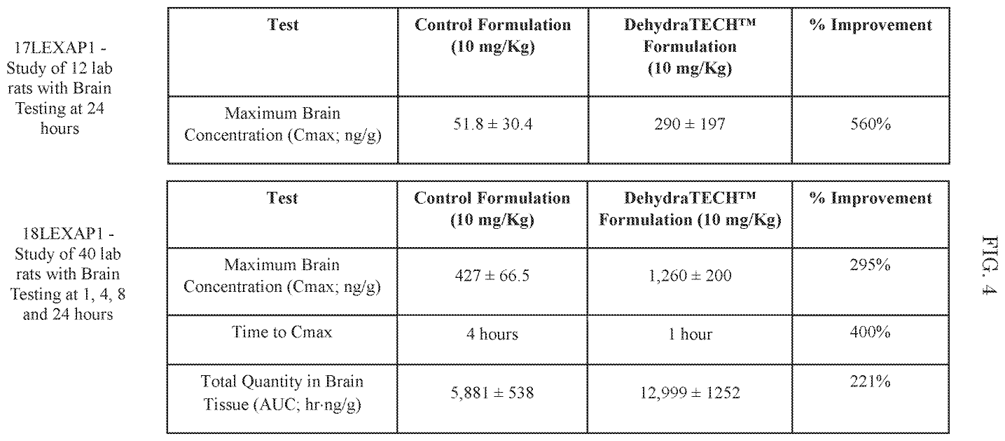

FIG. 4 shows results from Examples 5 and 6 comparing improvements in maximum brain concentration, time to Cmax, and total quantity in brain tissue following administration of DEHYDRATECH™ and control compositions in rats at various time points. Improvement by orders of magnitude were observed in the DEHYDRATECH™ compared to control formulations.

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

Although the foregoing subject matter has been described in some detail by way of illustration and example for purposes of clarity of understanding, it will be understood by those skilled in the art that certain changes and modifications can be practiced within the scope of the appended claims.

1.-34. (canceled)

35. A composition, comprising:
a) a lipophilic active agent;
b) an edible oil;
c) an effective amount of a bioavailability enhancing agent wherein the lipophilic active agent in a subject is at least 1.5 times greater than the bioavailability of the lipophilic active agent in the subject in the absence of the edible oil; and
d) an edible substrate;
wherein the composition enhances the delivery of lipophilic active agents across the blood-brain barrier.

36. The composition according to claim 35, comprising from 0.01 mg to 1,000 mg by weight of a lipophilic active agent.

37. The composition according to claim 36, wherein the lipophilic active agent is selected from the group consisting of: cannabinoids, terpenes and terpenoids, non-steroidal anti-inflammatory drugs (NSAIDs), vitamins, nicotine or an analog thereof, phosphodiesterase 5 (PDE5) inhibitors, Maca extract, hormones, fentanyl or an analog thereof, buprenorphine or an analog thereof, scopolamine or an analog thereof, antioxidants, a nicotine compound, and an imaging agent.

38. The composition according to claim 37, wherein the cannabinoid is a psychoactive cannabinoid.

39. The composition according to claim 37, wherein the cannabinoid is a non-psychoactive cannabinoid.

40. The composition according to claim 37, wherein the NSAID is acetylsalicylic acid, ibuprophen, acetaminophen, diclofenac, indomethacin, piroxicam, or a COX inhibitor.

41. The composition according to claim 35, wherein the concentration of lipophilic active agent in central nervous system tissue of the subject is at least 2 times greater than the concentration of lipophilic active agent in central nervous system tissue in the subject in the absence of the edible oil comprising long chain fatty acids and/or medium chain fatty acids.

42. The composition according to claim 35, wherein the concentration of lipophilic active agent in central nervous system tissue of the subject is at least 5 times greater than the concentration of lipophilic active agent in central nervous system tissue in the subject in the absence of the edible oil comprising long chain fatty acids and/or medium chain fatty acids.

43. The composition according to claim 35, wherein the edible product is a food product, wherein the edible substrate is selected from the group consisting of tea leaves, coffee beans, cocoa powder, meats, fish, fruits, vegetables, dairy products, legumes, pastas, breads, grains, seeds, nuts, spices, and herbs.

44. A process for making an edible product infused with a lipophilic active agent with enhanced delivery across the blood brain barrier in a subject, comprising:
a) providing a therapeutically effective amount of a lipophilic active agent;
b) providing a bioavailability enhancing agent;
c) providing an edible substrate;
d) contacting the edible substrate with an oil comprising the lipophilic active agent and the bioavailability enhancing agent; and
e) dehydrating the edible substrate;
thereby producing the edible product infused with a lipophilic active agent with enhanced delivery across the blood brain barrier in the subject; wherein the bioavailability enhancing agent comprises an edible oil comprising long chain fatty acids and/or medium chain fatty acids that enhance the bioavailability of the lipophilic active agent and enhance delivery across the blood brain barrier in the subject.

45. The process according to claim 44, wherein the edible product is selected from the group consisting of a pill, tablet, lozenge, mini lozenge, capsule, caplet, pouch, gum, spray, food, and combinations thereof.

46. The process according to claim 44, wherein the edible substrate is selected from the group consisting of inulin, starch, modified starches, xanthan gum, carboxymethyl cellulose, methyl cellulose, hydroxypropylmethyl cellulose, konjac, chitosan, tragacanth, karaya, ghatti, larch, carageenan, alginate, chemically modified alginate, agar, guar, locust bean, psyllium, tara, gellan, curdlan, pullan, gum arabic, gelatin, pectin, and combinations thereof.

47. The process according to claim 44, wherein the edible product further comprises an additive selected from the group consisting of a non-nicotine alkaloid, a mineral, a vitamin, a dietary supplement, a dietary mineral, a nutraceutical, an energizing agent, a soothing agent, a coloring agent, an amino acid, a chemsthetic agent, an antioxidant, a food grade emulsifier, a pH modifier, a botanical, a teeth whitening agent, a therapeutic agent, a sweetener, a flavorant, and combinations thereof.

48. The process according to claim 44, wherein the lipophilic active agent is selected from the group consisting of: cannabinoids, terpenes and terpenoids, non-steroidal anti-inflammatory drugs (NSAIDs), vitamins, nicotine or an analog thereof, phosphodiesterase 5 (PDE5) inhibitors, Maca extract, hormones, fentanyl or an analog thereof, buprenorphine or an analog thereof, scopolamine or an analog thereof, antioxidants, a nicotine compound, and an imaging agent.

49. The process according to claim 44, wherein the bioavailability of the lipophilic active agent in a subject is at least 2 times greater than the bioavailability of the lipophilic active agent in the subject in the absence of the edible oil comprising long chain fatty acids and/or medium chain fatty acids.

50. The process according to claim 44, wherein the bioavailability of the lipophilic active agent in a subject is at least 5 times greater than the bioavailability of the lipophilic active agent in the subject in the absence of the edible oil comprising long chain fatty acids and/or medium chain fatty acids.

51. The process according to claim 44, wherein the lipophilic active agent is a cannabinoid.

52. The process according to claim 44, wherein the lipophilic active agent is a nicotine compound is selected from the group consisting of nicotine, free base nicotine, pharmacologically acceptable salts of nicotine, a nicotine complex, and polymer resins of nicotine, wherein the polymer resin is selected from the group consisting of nicotine polacrilex and nicotine resinate.

53. The process according to claim 44, wherein the bioavailability enhancing agent is a protective colloid, an edible oil or fat, and a lipophilic active agent taste masking agent.

54. A process for making a beverage product infused with a lipophilic active agent obtainable by the steps of:
(i) providing an edible product infused with a lipophilic active agent wherein the lipophilic active agent is selected from the group consisting of cannabinoids, terpenes and terpenoids, non-steroidal anti-inflammatory drugs (NSAIDs), vitamins, nicotine or an analog thereof, phosphodiesterase 5 (PDE5) inhibitors, Maca extract, hormones, fentanyl, buprenorphine, scopolamine, antioxidants, a nicotine compound, and an imaging agent, wherein the edible product infused with a lipophilic active agent is tea leaves, coffee beans, or cocoa powder infused with a lipophilic active agent; and
(ii) steeping the tea leaves, coffee beans, or cocoa powder infused with a lipophilic active agent in a liquid;
thereby producing the beverage product infused with the lipophilic active agent.
